SEQUENCE LISTING (325 sequences)

SEQ ID NO: 1 - DNA, 132 bp, synthetic construct
  misc_feature 1..132: note = Sequence of Interest
  source 1..132: mol_type = other DNA
agccataagacgtactgcacctgttgggttacaatcatattctttcatgagagtcacgcatcccaatcgccccgtaacttccatcctttgacccgtcaaattgcttccccgtgttaacagcaacctccaaaa

SEQ ID NO: 2 - DNA, 16 bp, synthetic construct
  misc_feature 1..16: note = Oligo
  source 1..16: mol_type = other DNA
agccataagacgtact

SEQ ID NO: 3 - DNA, 16 bp, synthetic construct
  misc_feature 1..16: note = Oligo
  source 1..16: mol_type = other DNA
gtgcagtacgtcttat

SEQ ID NO: 4 - DNA, 16 bp, synthetic construct
  misc_feature 1..16: note = Oligo
  source 1..16: mol_type = other DNA
gcacctgttgggttac

SEQ ID NO: 5 - DNA, 16 bp, synthetic construct
  misc_feature 1..16: note = Oligo
  source 1..16: mol_type = other DNA
gattgtaacccaacag

SEQ ID NO: 6 - DNA, 16 bp, synthetic construct
  misc_feature 1..16: note = Oligo
  source 1..16: mol_type = other DNA
aatcatattctttcat

SEQ ID NO: 7 - DNA, 16 bp, synthetic construct
  misc_feature 1..16: note = Oligo
  source 1..16: mol_type = other DNA
tctcatgaaagaatat

SEQ ID NO: 8 - DNA, 16 bp, synthetic construct
  misc_feature 1..16: note = Oligo
  source 1..16: mol_type = other DNA
gagagtcacgcatccc

SEQ ID NO: 9 - DNA, 16 bp, synthetic construct
  misc_feature 1..16: note = Oligo
  source 1..16: mol_type = other DNA
gattgggatgcgtgac

SEQ ID NO: 10 - DNA, 16 bp, synthetic construct
  misc_feature 1..16: note = Oligo
  source 1..16: mol_type = other DNA
aatcgccccgtaactt

SEQ ID NO: 11 - DNA, 16 bp, synthetic construct
  misc_feature 1..16: note = Oligo
  source 1..16: mol_type = other DNA
atggaagttacggggc

SEQ ID NO: 12 - DNA, 16 bp, synthetic construct
  misc_feature 1..16: note = Oligo
  source 1..16: mol_type = other DNA
ccatcctttgacccgt

SEQ ID NO: 13 - DNA, 16 bp, synthetic construct
  misc_feature 1..16: note = Oligo
  source 1..16: mol_type = other DNA
tttgacgggtcaaagg

SEQ ID NO: 14 - DNA, 16 bp, synthetic construct
  misc_feature 1..16: note = Oligo
  source 1..16: mol_type = other DNA
caaattgcttccccgt

SEQ ID NO: 15 - DNA, 16 bp, synthetic construct
  misc_feature 1..16: note = Oligo
  source 1..16: mol_type = other DNA
taacacggggaagcaa

SEQ ID NO: 16 - DNA, 16 bp, synthetic construct
  misc_feature 1..16: note = Oligo
  source 1..16: mol_type = other DNA
gttaacagcaacctcc

SEQ ID NO: 17 - DNA, 16 bp, synthetic construct
  misc_feature 1..16: note = Oligo
  source 1..16: mol_type = other DNA
ttttggaggttgctgt

SEQ ID NO: 18 - DNA, 16 bp, synthetic construct
  misc_feature 1..16: note = Oligo
  source 1..16: mol_type = other DNA
atagtatcgagcgacc

SEQ ID NO: 19 - DNA, 16 bp, synthetic construct
  misc_feature 1..16: note = Oligo
  source 1..16: mol_type = other DNA
gattggtcgctcgata

SEQ ID NO: 20 - DNA, 16 bp, synthetic construct
  misc_feature 1..16: note = Oligo
  source 1..16: mol_type = other DNA
aatcgcatcggcatat

SEQ ID NO: 21 - DNA, 16 bp, synthetic construct
  misc_feature 1..16: note = Oligo
  source 1..16: mol_type = other DNA
ctatatatgccgatgc

SEQ ID NO: 22 - DNA, 16 bp, synthetic construct
  misc_feature 1..16: note = Oligo
  source 1..16: mol_type = other DNA
atagtatggccatatg

SEQ ID NO: 23 - DNA, 16 bp, synthetic construct
  misc_feature 1..16: note = Oligo
  source 1..16: mol_type = other DNA
tgagcatatggccata

SEQ ID NO: 24 - DNA, 16 bp, synthetic construct
  misc_feature 1..16: note = Oligo
  source 1..16: mol_type = other DNA
ctcattgagagtactg

SEQ ID NO: 25 - DNA, 16 bp, synthetic construct
  misc_feature 1..16: note = Oligo
  source 1..16: mol_type = other DNA
gtcacagtactctcaa

SEQ ID NO: 26 - DNA, 16 bp, synthetic construct
  misc_feature 1..16: note = Oligo
  source 1..16: mol_type = other DNA
atgttatcgagcgacc

SEQ ID NO: 27 - DNA, 260 bp, synthetic construct
  misc_feature 1..260: note = Sequence of Interest
  source 1..260: mol_type = other DNA
gcaccaaacgaaaatactatgggccaacgataccgagtgtacggacacagacgattacgtctgaatctcatgcccagtagtcggaggcgcggccagagatattgaggcgatcagccacccgaatggagaacaaagccacaacccgtattgcgtctctatttgttttgattgccgcatcatctagtgatgagggcacctgaacaccttaatacctaattcgtcaccacattgttgtgagccatagggggacatcctgttta

SEQ ID NO: 28 - DNA, 16 bp, synthetic construct
  misc_feature 1..16: note = Oligo
  source 1..16: mol_type = other DNA
gcaccaaacgaaaata

SEQ ID NO: 29 - DNA, 16 bp, synthetic construct
  misc_feature 1..16: note = Oligo
  source 1..16: mol_type = other DNA
atagtattttcgtttg

SEQ ID NO: 30 - DNA, 16 bp, synthetic construct
  misc_feature 1..16: note = Oligo
  source 1..16: mol_type = other DNA
ctatgggccaacgata

SEQ ID NO: 31 - DNA, 16 bp, synthetic construct
  misc_feature 1..16: note = Oligo
  source 1..16: mol_type = other DNA
tcggtatcgttggccc

SEQ ID NO: 32 - DNA, 16 bp, synthetic construct
  misc_feature 1..16: note = Oligo
  source 1..16: mol_type = other DNA
ccgagtgtacggacac

SEQ ID NO: 33 - DNA, 16 bp, synthetic construct
  misc_feature 1..16: note = Oligo
  source 1..16: mol_type = other DNA
gtctgtgtccgtacac

SEQ ID NO: 34 - DNA, 16 bp, synthetic construct
  misc_feature 1..16: note = Oligo
  source 1..16: mol_type = other DNA
agacgattacgtctga

SEQ ID NO: 35 - DNA, 16 bp, synthetic construct
  misc_feature 1..16: note = Oligo
  source 1..16: mol_type = other DNA
agattcagacgtaatc

SEQ ID NO: 36 - DNA, 16 bp, synthetic construct
  misc_feature 1..16: note = Oligo
  source 1..16: mol_type = other DNA
atctcatgcccagtag

SEQ ID NO: 37 - DNA, 16 bp, synthetic construct
  misc_feature 1..16: note = Oligo
  source 1..16: mol_type = other DNA
ccgactactgggcatg

SEQ ID NO: 38 - DNA, 16 bp, synthetic construct
  misc_feature 1..16: note = Oligo
  source 1..16: mol_type = other DNA
tcggaggcgcggccag

SEQ ID NO: 39 - DNA, 16 bp, synthetic construct
  misc_feature 1..16: note = Oligo
  source 1..16: mol_type = other DNA
atctctggccgcgcct

SEQ ID NO: 40 - DNA, 16 bp, synthetic construct
  misc_feature 1..16: note = Oligo
  source 1..16: mol_type = other DNA
agatattgaggcgatc

SEQ ID NO: 41 - DNA, 16 bp, synthetic construct
  misc_feature 1..16: note = Oligo
  source 1..16: mol_type = other DNA
ggctgatcgcctcaat

SEQ ID NO: 42 - DNA, 16 bp, synthetic construct
  misc_feature 1..16: note = Oligo
  source 1..16: mol_type = other DNA
agccacccgaatggag

SEQ ID NO: 43 - DNA, 16 bp, synthetic construct
  misc_feature 1..16: note = Oligo
  source 1..16: mol_type = other DNA
tgttctccattcgggt

SEQ ID NO: 44 - DNA, 16 bp, synthetic construct
  misc_feature 1..16: note = Oligo
  source 1..16: mol_type = other DNA
aacaaagccacaaccc

SEQ ID NO: 45 - DNA, 16 bp, synthetic construct
  misc_feature 1..16: note = Oligo
  source 1..16: mol_type = other DNA
atacgggttgtggctt

SEQ ID NO: 46 - DNA, 16 bp, synthetic construct
  misc_feature 1..16: note = Oligo
  source 1..16: mol_type = other DNA
gtattgcgtctctatt

SEQ ID NO: 47 - DNA, 16 bp, synthetic construct
  misc_feature 1..16: note = Oligo
  source 1..16: mol_type = other DNA
aacaaatagagacgca

SEQ ID NO: 48 - DNA, 16 bp, synthetic construct
  misc_feature 1..16: note = Oligo
  source 1..16: mol_type = other DNA
tgttttgattgccgca

SEQ ID NO: 49 - DNA, 16 bp, synthetic construct
  misc_feature 1..16: note = Oligo
  source 1..16: mol_type = other DNA
atgatgcggcaatcaa

SEQ ID NO: 50 - DNA, 16 bp, synthetic construct
  misc_feature 1..16: note = Oligo
  source 1..16: mol_type = other DNA
tcatctagtgatgagg

SEQ ID NO: 51 - DNA, 16 bp, synthetic construct
  misc_feature 1..16: note = Oligo
  source 1..16: mol_type = other DNA
gtgccctcatcactag

SEQ ID NO: 52 - DNA, 16 bp, synthetic construct
  misc_feature 1..16: note = Oligo
  source 1..16: mol_type = other DNA
gcacctgaacacctta

SEQ ID NO: 53 - DNA, 16 bp, synthetic construct
  misc_feature 1..16: note = Oligo
  source 1..16: mol_type = other DNA
gtattaaggtgttcag

SEQ ID NO: 54 - DNA, 16 bp, synthetic construct
  misc_feature 1..16: note = Oligo
  source 1..16: mol_type = other DNA
atacctaattcgtcac

SEQ ID NO: 55 - DNA, 16 bp, synthetic construct
  misc_feature 1..16: note = Oligo
  source 1..16: mol_type = other DNA
tgtggtgacgaattag

SEQ ID NO: 56 - DNA, 16 bp, synthetic construct
  misc_feature 1..16: note = Oligo
  source 1..16: mol_type = other DNA
cacattgttgtgagcc

SEQ ID NO: 57 - DNA, 16 bp, synthetic construct
  misc_feature 1..16: note = Oligo
  source 1..16: mol_type = other DNA
ctatggctcacaacaa

SEQ ID NO: 58 - DNA, 16 bp, synthetic construct
  misc_feature 1..16: note = Oligo
  source 1..16: mol_type = other DNA
atagggggacatcctg

SEQ ID NO: 59 - DNA, 16 bp, synthetic construct
  misc_feature 1..16: note = Oligo
  source 1..16: mol_type = other DNA
taaacaggatgtcccc

SEQ ID NO: 60 - DNA, 1028 bp, synthetic construct
  misc_feature 1..1028: note = Sequence of Interest
  source 1..1028: mol_type = other DNA
cacttagtgctcgtcccgttgatcgctggtgcatcagggccttgagctggcctgttggtgggcctgctaacgccgccctcctgtggacgaagtggaatgagagacgtgcgccatgttatctccgccagccgtccatgcgtgaccgggtcgggtacgaccgggccggaacggactcacacccatggtgagcaatacgtatccaccgggagcttagtttggcccccttgggccgacccggggagtccgaacgctcgggactactacttcacgagcaagcatctccggcgccgctgcccgcgatctggcacgccccttccagactcctgggcagcgacagcgccgagagggcggtcgcccaggggaaccgccctacagcccccgagatcgtttctccccccgcttccacccctgggcgaggtgcatacctttcaacccgtcaaatgccacttagaacgtcctgctcgtccgtagaaaacaggcagacgccggacgcccatatgggaggcgctcgttggcccgctgggcgactgggcgtgactggcgaccgccaggtatccccggggggagcgcgccacgctgcaggcggacgcgagggagatgcagggacggggtggtcagcttgacccgggcctgggaggctcgtccggcccctcgcggctagactgggatagcggtataccagcgggaggagtctctccggcgtccggccctgcaggcaaaggcgtgagcgtttcccgctgtccgggcggtagcggctggggccatgtccgacggcgcgcgatgtagtcggggcccattccacggtgcaacgtccccctacagtaggtcaatcggcaggcacaggcgcgcgcgcccgccgcgctagttccgtggccgtcgtacagagcagaggaatccgaagtcgggttgcccgggctcctgtccggccaccgccccgagctccctacgggacagcccgggaaagctgggagggacagggtagtcggcccccccgctctgtcgttccgcatccgccttgcgggccccgc

SEQ ID NO: 61 - DNA, 11 bp, synthetic construct
  misc_feature 1..11: note = Oligo
  source 1..11: mol_type = other DNA
tcgaggaacgc

SEQ ID NO: 62 - DNA, 15 bp, synthetic construct
  misc_feature 1..15: note = Oligo
  source 1..15: mol_type = other DNA
atatgcgttcctcga

SEQ ID NO: 63 - DNA, 11 bp, synthetic construct
  misc_feature 1..11: note = Oligo
  source 1..11: mol_type = other DNA
atatcggtaaa

SEQ ID NO: 64 - skipped

SEQ ID NO: 65 - DNA, 10 bp, synthetic construct
  misc_feature 1..10: note = Oligo
  source 1..10: mol_type = other DNA
tcgaggaacg

SEQ ID NO: 66 - DNA, 14 bp, synthetic construct
  misc_feature 1..14: note = Oligo
  source 1..14: mol_type = other DNA
tatgcgttcctcga

SEQ ID NO: 67 - DNA, 12 bp, synthetic construct
  misc_feature 1..12: note = Oligo
  source 1..12: mol_type = other DNA
catatcggtaaa

SEQ ID NO: 68 - skipped

SEQ ID NO: 69 - DNA, 12 bp, synthetic construct
  misc_feature 1..12: note = Oligo
  source 1..12: mol_type = other DNA
tcgaggaacgca

SEQ ID NO: 70 - DNA, 10 bp, synthetic construct
  misc_feature 1..10: note = Oligo
  source 1..10: mol_type = other DNA
tatcggtaaa

SEQ ID NO: 71 - DNA, 10 bp, synthetic construct
  misc_feature 1..10: note = Oligo
  source 1..10: mol_type = other DNA
tcgaggaacg

SEQ ID NO: 72 - DNA, 12 bp, synthetic construct
  misc_feature 1..12: note = Oligo
  source 1..12: mol_type = other DNA
catatcggtaaa

SEQ ID NO: 73 - DNA, 16 bp, synthetic construct
  misc_feature 1..16: note = Oligo
  source 1..16: mol_type = other DNA
cacttagtgctcgtcc

SEQ ID NO: 74 - DNA, 16 bp, synthetic construct
  misc_feature 1..16: note = Oligo
  source 1..16: mol_type = other DNA
cgttgatcgctggtgc

SEQ ID NO: 75 - DNA, 16 bp, synthetic construct
  misc_feature 1..16: note = Oligo
  source 1..16: mol_type = other DNA
atcagggccttgagct

SEQ ID NO: 76 - DNA, 16 bp, synthetic construct
  misc_feature 1..16: note = Oligo
  source 1..16: mol_type = other DNA
ggcctgttggtgggcc

SEQ ID NO: 77 - DNA, 16 bp, synthetic construct
  misc_feature 1..16: note = Oligo
  source 1..16: mol_type = other DNA
tgctaacgccgccctc

SEQ ID NO: 78 - DNA, 16 bp, synthetic construct
  misc_feature 1..16: note = Oligo
  source 1..16: mol_type = other DNA
ctgtggacgaagtgga

SEQ ID NO: 79 - DNA, 16 bp, synthetic construct
  misc_feature 1..16: note = Oligo
  source 1..16: mol_type = other DNA
atgagagacgtgcgcc

SEQ ID NO: 80 - DNA, 16 bp, synthetic construct
  misc_feature 1..16: note = Oligo
  source 1..16: mol_type = other DNA
atgttatctccgccag

SEQ ID NO: 81 - DNA, 16 bp, synthetic construct
  misc_feature 1..16: note = Oligo
  source 1..16: mol_type = other DNA
ccgtccatgcgtgacc

SEQ ID NO: 82 - DNA, 16 bp, synthetic construct
  misc_feature 1..16: note = Oligo
  source 1..16: mol_type = other DNA
gggtcgggtacgaccg

SEQ ID NO: 83 - DNA, 16 bp, synthetic construct
  misc_feature 1..16: note = Oligo
  source 1..16: mol_type = other DNA
ggccggaacggactca

SEQ ID NO: 84 - DNA, 16 bp, synthetic construct
  misc_feature 1..16: note = Oligo
  source 1..16: mol_type = other DNA
cacccatggtgagcaa

SEQ ID NO: 85 - DNA, 16 bp, synthetic construct
  misc_feature 1..16: note = Oligo
  source 1..16: mol_type = other DNA
tacgtatccaccggga

SEQ ID NO: 86 - DNA, 16 bp, synthetic construct
  misc_feature 1..16: note = Oligo
  source 1..16: mol_type = other DNA
gcttagtttggccccc

SEQ ID NO: 87 - DNA, 16 bp, synthetic construct
  misc_feature 1..16: note = Oligo
  source 1..16: mol_type = other DNA
ttgggccgacccgggg

SEQ ID NO: 88 - DNA, 16 bp, synthetic construct
  misc_feature 1..16: note = Oligo
  source 1..16: mol_type = other DNA
agtccgaacgctcggg

SEQ ID NO: 89 - DNA, 16 bp, synthetic construct
  misc_feature 1..16: note = Oligo
  source 1..16: mol_type = other DNA
actactacttcacgag

SEQ ID NO: 90 - DNA, 16 bp, synthetic construct
  misc_feature 1..16: note = Oligo
  source 1..16: mol_type = other DNA
caagcatctccggcgc

SEQ ID NO: 91 - DNA, 16 bp, synthetic construct
  misc_feature 1..16: note = Oligo
  source 1..16: mol_type = other DNA
cgctgcccgcgatctg

SEQ ID NO: 92 - DNA, 16 bp, synthetic construct
  misc_feature 1..16: note = Oligo
  source 1..16: mol_type = other DNA
gcacgccccttccaga

SEQ ID NO: 93 - DNA, 16 bp, synthetic construct
  misc_feature 1..16: note = Oligo
  source 1..16: mol_type = other DNA
ctcctgggcagcgaca

SEQ ID NO: 94 - DNA, 16 bp, synthetic construct
  misc_feature 1..16: note = Oligo
  source 1..16: mol_type = other DNA
gcgccgagagggcggt

SEQ ID NO: 95 - DNA, 16 bp, synthetic construct
  misc_feature 1..16: note = Oligo
  source 1..16: mol_type = other DNA
cgcccaggggaaccgc

SEQ ID NO: 96 - DNA, 16 bp, synthetic construct
  misc_feature 1..16: note = Oligo
  source 1..16: mol_type = other DNA
cctacagcccccgaga

SEQ ID NO: 97 - DNA, 16 bp, synthetic construct
  misc_feature 1..16: note = Oligo
  source 1..16: mol_type = other DNA
tcgtttctccccccgc

SEQ ID NO: 98 - DNA, 16 bp, synthetic construct
  misc_feature 1..16: note = Oligo
  source 1..16: mol_type = other DNA
ttccacccctgggcga

SEQ ID NO: 99 - DNA, 16 bp, synthetic construct
  misc_feature 1..16: note = Oligo
  source 1..16: mol_type = other DNA
ggtgcatacctttcaa

SEQ ID NO: 100 - DNA, 16 bp, synthetic construct
  misc_feature 1..16: note = Oligo
  source 1..16: mol_type = other DNA
cccgtcaaatgccact

SEQ ID NO: 101 - DNA, 16 bp, synthetic construct
  misc_feature 1..16: note = Oligo
  source 1..16: mol_type = other DNA
tagaacgtcctgctcg

SEQ ID NO: 102 - DNA, 16 bp, synthetic construct
  misc_feature 1..16: note = Oligo
  source 1..16: mol_type = other DNA
tccgtagaaaacaggc

SEQ ID NO: 103 - DNA, 16 bp, synthetic construct
  misc_feature 1..16: note = Oligo
  source 1..16: mol_type = other DNA
agacgccggacgccca

SEQ ID NO: 104 - DNA, 16 bp, synthetic construct
  misc_feature 1..16: note = Oligo
  source 1..16: mol_type = other DNA
tatgggaggcgctcgt

SEQ ID NO: 105 - DNA, 16 bp, synthetic construct
  misc_feature 1..16: note = Oligo
  source 1..16: mol_type = other DNA
tggcccgctgggcgac

SEQ ID NO: 106 - DNA, 16 bp, synthetic construct
  misc_feature 1..16: note = Oligo
  source 1..16: mol_type = other DNA
tgggcgtgactggcga

SEQ ID NO: 107 - DNA, 16 bp, synthetic construct
  misc_feature 1..16: note = Oligo
  source 1..16: mol_type = other DNA
ccgccaggtatccccg

SEQ ID NO: 108 - DNA, 16 bp, synthetic construct
  misc_feature 1..16: note = Oligo
  source 1..16: mol_type = other DNA
gggggagcgcgccacg

SEQ ID NO: 109 - DNA, 16 bp, synthetic construct
  misc_feature 1..16: note = Oligo
  source 1..16: mol_type = other DNA
ctgcaggcggacgcga

SEQ ID NO: 110 - DNA, 16 bp, synthetic construct
  misc_feature 1..16: note = Oligo
  source 1..16: mol_type = other DNA
gggagatgcagggacg

SEQ ID NO: 111 - DNA, 16 bp, synthetic construct
  misc_feature 1..16: note = Oligo
  source 1..16: mol_type = other DNA
gggtggtcagcttgac

SEQ ID NO: 112 - DNA, 16 bp, synthetic construct
  misc_feature 1..16: note = Oligo
  source 1..16: mol_type = other DNA
ccgggcctgggaggct

SEQ ID NO: 113 - DNA, 16 bp, synthetic construct
  misc_feature 1..16: note = Oligo
  source 1..16: mol_type = other DNA
cgtccggcccctcgcg

SEQ ID NO: 114 - DNA, 16 bp, synthetic construct
  misc_feature 1..16: note = Oligo
  source 1..16: mol_type = other DNA
gctagactgggatagc

SEQ ID NO: 115 - DNA, 16 bp, synthetic construct
  misc_feature 1..16: note = Oligo
  source 1..16: mol_type = other DNA
ggtataccagcgggag

SEQ ID NO: 116 - DNA, 16 bp, synthetic construct
  misc_feature 1..16: note = Oligo
  source 1..16: mol_type = other DNA
gagtctctccggcgtc

SEQ ID NO: 117 - DNA, 16 bp, synthetic construct
  misc_feature 1..16: note = Oligo
  source 1..16: mol_type = other DNA
cggccctgcaggcaaa

SEQ ID NO: 118 - DNA, 16 bp, synthetic construct
  misc_feature 1..16: note = Oligo
  source 1..16: mol_type = other DNA
ggcgtgagcgtttccc

SEQ ID NO: 119 - DNA, 16 bp, synthetic construct
  misc_feature 1..16: note = Oligo
  source 1..16: mol_type = other DNA
gctgtccgggcggtag

SEQ ID NO: 120 - DNA, 16 bp, synthetic construct
  misc_feature 1..16: note = Oligo
  source 1..16: mol_type = other DNA
cggctggggccatgtc

SEQ ID NO: 121 - DNA, 16 bp, synthetic construct
  misc_feature 1..16: note = Oligo
  source 1..16: mol_type = other DNA
cgacggcgcgcgatgt

SEQ ID NO: 122 - DNA, 16 bp, synthetic construct
  misc_feature 1..16: note = Oligo
  source 1..16: mol_type = other DNA
agtcggggcccattcc

SEQ ID NO: 123 - DNA, 16 bp, synthetic construct
  misc_feature 1..16: note = Oligo
  source 1..16: mol_type = other DNA
acggtgcaacgtcccc

SEQ ID NO: 124 - DNA, 16 bp, synthetic construct
  misc_feature 1..16: note = Oligo
  source 1..16: mol_type = other DNA
ctacagtaggtcaatc

SEQ ID NO: 125 - DNA, 16 bp, synthetic construct
  misc_feature 1..16: note = Oligo
  source 1..16: mol_type = other DNA
ggcaggcacaggcgcg

SEQ ID NO: 126 - DNA, 16 bp, synthetic construct
  misc_feature 1..16: note = Oligo
  source 1..16: mol_type = other DNA
cgcgcccgccgcgcta

SEQ ID NO: 127 - DNA, 16 bp, synthetic construct
  misc_feature 1..16: note = Oligo
  source 1..16: mol_type = other DNA
gttccgtggccgtcgt

SEQ ID NO: 128 - DNA, 16 bp, synthetic construct
  misc_feature 1..16: note = Oligo
  source 1..16: mol_type = other DNA
acagagcagaggaatc

SEQ ID NO: 129 - DNA, 16 bp, synthetic construct
  misc_feature 1..16: note = Oligo
  source 1..16: mol_type = other DNA
cgaagtcgggttgccc

SEQ ID NO: 130 - DNA, 16 bp, synthetic construct
  misc_feature 1..16: note = Oligo
  source 1..16: mol_type = other DNA
gggctcctgtccggcc

SEQ ID NO: 131 - DNA, 16 bp, synthetic construct
  misc_feature 1..16: note = Oligo
  source 1..16: mol_type = other DNA
accgccccgagctccc

SEQ ID NO: 132 - DNA, 16 bp, synthetic construct
  misc_feature 1..16: note = Oligo
  source 1..16: mol_type = other DNA
tacgggacagcccggg

SEQ ID NO: 133 - DNA, 16 bp, synthetic construct
  misc_feature 1..16: note = Oligo
  source 1..16: mol_type = other DNA
aaagctgggagggaca

SEQ ID NO: 134 - DNA, 16 bp, synthetic construct
  misc_feature 1..16: note = Oligo
  source 1..16: mol_type = other DNA
gggtagtcggcccccc

SEQ ID NO: 135 - DNA, 16 bp, synthetic construct
  misc_feature 1..16: note = Oligo
  source 1..16: mol_type = other DNA
cgctctgtcgttccgc

SEQ ID NO: 136 - DNA, 16 bp, synthetic construct
  misc_feature 1..16: note = Oligo
  source 1..16: mol_type = other DNA
atccgccttgcgggcc

SEQ ID NO: 137 - DNA, 16 bp, synthetic construct
  misc_feature 1..16: note = Oligo
  source 1..16: mol_type = other DNA
aacgggacgagcacta

SEQ ID NO: 138 - DNA, 16 bp, synthetic construct
  misc_feature 1..16: note = Oligo
  source 1..16: mol_type = other DNA
tgatgcaccagcgatc

SEQ ID NO: 139 - DNA, 16 bp, synthetic construct
  misc_feature 1..16: note = Oligo
  source 1..16: mol_type = other DNA
ggccagctcaaggccc

SEQ ID NO: 140 - DNA, 16 bp, synthetic construct
  misc_feature 1..16: note = Oligo
  source 1..16: mol_type = other DNA
agcaggcccaccaaca

SEQ ID NO: 141 - DNA, 16 bp, synthetic construct
  misc_feature 1..16: note = Oligo
  source 1..16: mol_type = other DNA
acaggagggcggcgtt

SEQ ID NO: 142 - DNA, 16 bp, synthetic construct
  misc_feature 1..16: note = Oligo
  source 1..16: mol_type = other DNA
tcattccacttcgtcc

SEQ ID NO: 143 - DNA, 16 bp, synthetic construct
  misc_feature 1..16: note = Oligo
  source 1..16: mol_type = other DNA
acatggcgcacgtctc

SEQ ID NO: 144 - DNA, 16 bp, synthetic construct
  misc_feature 1..16: note = Oligo
  source 1..16: mol_type = other DNA
acggctggcggagata

SEQ ID NO: 145 - DNA, 16 bp, synthetic construct
  misc_feature 1..16: note = Oligo
  source 1..16: mol_type = other DNA
acccggtcacgcatgg

SEQ ID NO: 146 - DNA, 16 bp, synthetic construct
  misc_feature 1..16: note = Oligo
  source 1..16: mol_type = other DNA
ggcccggtcgtacccg

SEQ ID NO: 147 - DNA, 16 bp, synthetic construct
  misc_feature 1..16: note = Oligo
  source 1..16: mol_type = other DNA
ggtgtgagtccgttcc

SEQ ID NO: 148 - DNA, 16 bp, synthetic construct
  misc_feature 1..16: note = Oligo
  source 1..16: mol_type = other DNA
cgtattgctcaccatg

SEQ ID NO: 149 - DNA, 16 bp, synthetic construct
  misc_feature 1..16: note = Oligo
  source 1..16: mol_type = other DNA
aagctcccggtggata

SEQ ID NO: 150 - DNA, 16 bp, synthetic construct
  misc_feature 1..16: note = Oligo
  source 1..16: mol_type = other DNA
ccaagggggccaaact

SEQ ID NO: 151 - DNA, 16 bp, synthetic construct
  misc_feature 1..16: note = Oligo
  source 1..16: mol_type = other DNA
gactccccgggtcggc

SEQ ID NO: 152 - DNA, 16 bp, synthetic construct
  misc_feature 1..16: note = Oligo
  source 1..16: mol_type = other DNA
tagtcccgagcgttcg

SEQ ID NO: 153 - DNA, 16 bp, synthetic construct
  misc_feature 1..16: note = Oligo
  source 1..16: mol_type = other DNA
cttgctcgtgaagtag

SEQ ID NO: 154 - DNA, 16 bp, synthetic construct
  misc_feature 1..16: note = Oligo
  source 1..16: mol_type = other DNA
agcggcgccggagatg

SEQ ID NO: 155 - DNA, 16 bp, synthetic construct
  misc_feature 1..16: note = Oligo
  source 1..16: mol_type = other DNA
gtgccagatcgcgggc

SEQ ID NO: 156 - DNA, 16 bp, synthetic construct
  misc_feature 1..16: note = Oligo
  source 1..16: mol_type = other DNA
ggagtctggaaggggc

SEQ ID NO: 157 - DNA, 16 bp, synthetic construct
  misc_feature 1..16: note = Oligo
  source 1..16: mol_type = other DNA
gcgctgtcgctgccca

SEQ ID NO: 158 - DNA, 16 bp, synthetic construct
  misc_feature 1..16: note = Oligo
  source 1..16: mol_type = other DNA
ggcgaccgccctctcg

SEQ ID NO: 159 - DNA, 16 bp, synthetic construct
  misc_feature 1..16: note = Oligo
  source 1..16: mol_type = other DNA
tagggcggttcccctg

SEQ ID NO: 160 - DNA, 16 bp, synthetic construct
  misc_feature 1..16: note = Oligo
  source 1..16: mol_type = other DNA
acgatctcgggggctg

SEQ ID NO: 161 - DNA, 16 bp, synthetic construct
  misc_feature 1..16: note = Oligo
  source 1..16: mol_type = other DNA
ggaagcggggggagaa

SEQ ID NO: 162 - DNA, 16 bp, synthetic construct
  misc_feature 1..16: note = Oligo
  source 1..16: mol_type = other DNA
cacctcgcccaggggt

SEQ ID NO: 163 - DNA, 16 bp, synthetic construct
  misc_feature 1..16: note = Oligo
  source 1..16: mol_type = other DNA
cgggttgaaaggtatg

SEQ ID NO: 164 - DNA, 16 bp, synthetic construct
  misc_feature 1..16: note = Oligo
  source 1..16: mol_type = other DNA
tctaagtggcatttga

SEQ ID NO: 165 - DNA, 16 bp, synthetic construct
  misc_feature 1..16: note = Oligo
  source 1..16: mol_type = other DNA
cggacgagcaggacgt

SEQ ID NO: 166 - DNA, 16 bp, synthetic construct
  misc_feature 1..16: note = Oligo
  source 1..16: mol_type = other DNA
gtctgcctgttttcta

SEQ ID NO: 167 - DNA, 16 bp, synthetic construct
  misc_feature 1..16: note = Oligo
  source 1..16: mol_type = other DNA
catatgggcgtccggc

SEQ ID NO: 168 - DNA, 16 bp, synthetic construct
  misc_feature 1..16: note = Oligo
  source 1..16: mol_type = other DNA
gccaacgagcgcctcc

SEQ ID NO: 169 - DNA, 16 bp, synthetic construct
  misc_feature 1..16: note = Oligo
  source 1..16: mol_type = other DNA
cccagtcgcccagcgg

SEQ ID NO: 170 - DNA, 16 bp, synthetic construct
  misc_feature 1..16: note = Oligo
  source 1..16: mol_type = other DNA
gcggtcgccagtcacg

SEQ ID NO: 171 - DNA, 16 bp, synthetic construct
  misc_feature 1..16: note = Oligo
  source 1..16: mol_type = other DNA
cccccggggatacctg

SEQ ID NO: 172 - DNA, 16 bp, synthetic construct
  misc_feature 1..16: note = Oligo
  source 1..16: mol_type = other DNA
gcagcgtggcgcgctc

SEQ ID NO: 173 - DNA, 16 bp, synthetic construct
  misc_feature 1..16: note = Oligo
  source 1..16: mol_type = other DNA
tccctcgcgtccgcct

SEQ ID NO: 174 - DNA, 16 bp, synthetic construct
  misc_feature 1..16: note = Oligo
  source 1..16: mol_type = other DNA
accccgtccctgcatc

SEQ ID NO: 175 - DNA, 16 bp, synthetic construct
  misc_feature 1..16: note = Oligo
  source 1..16: mol_type = other DNA
ccgggtcaagctgacc

SEQ ID NO: 176 - DNA, 16 bp, synthetic construct
  misc_feature 1..16: note = Oligo
  source 1..16: mol_type = other DNA
gacgagcctcccaggc

SEQ ID NO: 177 - DNA, 16 bp, synthetic construct
  misc_feature 1..16: note = Oligo
  source 1..16: mol_type = other DNA
tagccgcgaggggccg

SEQ ID NO: 178 - DNA, 16 bp, synthetic construct
  misc_feature 1..16: note = Oligo
  source 1..16: mol_type = other DNA
taccgctatcccagtc

SEQ ID NO: 179 - DNA, 16 bp, synthetic construct
  misc_feature 1..16: note = Oligo
  source 1..16: mol_type = other DNA
actcctcccgctggta

SEQ ID NO: 180 - DNA, 16 bp, synthetic construct
  misc_feature 1..16: note = Oligo
  source 1..16: mol_type = other DNA
gccggacgccggagag

SEQ ID NO: 181 - DNA, 16 bp, synthetic construct
  misc_feature 1..16: note = Oligo
  source 1..16: mol_type = other DNA
cgcctttgcctgcagg

SEQ ID NO: 182 - DNA, 16 bp, synthetic construct
  misc_feature 1..16: note = Oligo
  source 1..16: mol_type = other DNA
cagcgggaaacgctca

SEQ ID NO: 183 - DNA, 16 bp, synthetic construct
  misc_feature 1..16: note = Oligo
  source 1..16: mol_type = other DNA
gccgctaccgcccgga

SEQ ID NO: 184 - DNA, 16 bp, synthetic construct
  misc_feature 1..16: note = Oligo
  source 1..16: mol_type = other DNA
gtcggacatggcccca

SEQ ID NO: 185 - DNA, 16 bp, synthetic construct
  misc_feature 1..16: note = Oligo
  source 1..16: mol_type = other DNA
gactacatcgcgcgcc

SEQ ID NO: 186 - DNA, 16 bp, synthetic construct
  misc_feature 1..16: note = Oligo
  source 1..16: mol_type = other DNA
ccgtggaatgggcccc

SEQ ID NO: 187 - DNA, 16 bp, synthetic construct
  misc_feature 1..16: note = Oligo
  source 1..16: mol_type = other DNA
gtagggggacgttgca

SEQ ID NO: 188 - DNA, 16 bp, synthetic construct
  misc_feature 1..16: note = Oligo
  source 1..16: mol_type = other DNA
tgccgattgacctact

SEQ ID NO: 189 - DNA, 16 bp, synthetic construct
  misc_feature 1..16: note = Oligo
  source 1..16: mol_type = other DNA
cgcgcgcgcctgtgcc

SEQ ID NO: 190 - DNA, 16 bp, synthetic construct
  misc_feature 1..16: note = Oligo
  source 1..16: mol_type = other DNA
gaactagcgcggcggg

SEQ ID NO: 191 - DNA, 16 bp, synthetic construct
  misc_feature 1..16: note = Oligo
  source 1..16: mol_type = other DNA
ctgtacgacggccacg

SEQ ID NO: 192 - DNA, 16 bp, synthetic construct
  misc_feature 1..16: note = Oligo
  source 1..16: mol_type = other DNA
ttcggattcctctgct

SEQ ID NO: 193 - DNA, 16 bp, synthetic construct
  misc_feature 1..16: note = Oligo
  source 1..16: mol_type = other DNA
gcccgggcaacccgac

SEQ ID NO: 194 - DNA, 16 bp, synthetic construct
  misc_feature 1..16: note = Oligo
  source 1..16: mol_type = other DNA
cggtggccggacagga

SEQ ID NO: 195 - DNA, 16 bp, synthetic construct
  misc_feature 1..16: note = Oligo
  source 1..16: mol_type = other DNA
cgtagggagctcgggg

SEQ ID NO: 196 - DNA, 16 bp, synthetic construct
  misc_feature 1..16: note = Oligo
  source 1..16: mol_type = other DNA
ctttcccgggctgtcc

SEQ ID NO: 197 - DNA, 16 bp, synthetic construct
  misc_feature 1..16: note = Oligo
  source 1..16: mol_type = other DNA
accctgtccctcccag

SEQ ID NO: 198 - DNA, 16 bp, synthetic construct
  misc_feature 1..16: note = Oligo
  source 1..16: mol_type = other DNA
agcgggggggccgact

SEQ ID NO: 199 - DNA, 16 bp, synthetic construct
  misc_feature 1..16: note = Oligo
  source 1..16: mol_type = other DNA
ggatgcggaacgacag

SEQ ID NO: 200 - DNA, 16 bp, synthetic construct
  misc_feature 1..16: note = Oligo
  source 1..16: mol_type = other DNA
gcggggcccgcaaggc

SEQ ID NO: 201 - DNA, 15 bp, synthetic construct
  misc_feature 1..15: note = Oligo
  source 1..15: mol_type = other DNA
cacttagtgctcgtc

SEQ ID NO: 202 - DNA, 15 bp, synthetic construct
  misc_feature 1..15: note = Oligo
  source 1..15: mol_type = other DNA
ccgttgatcgctggt

SEQ ID NO: 203 - DNA, 17 bp, synthetic construct
  misc_feature 1..17: note = Oligo
  source 1..17: mol_type = other DNA
gcatcagggccttgagc

SEQ ID NO: 204 - DNA, 16 bp, synthetic construct
  misc_feature 1..16: note = Oligo
  source 1..16: mol_type = other DNA
tggcctgttggtgggc

SEQ ID NO: 205 - DNA, 16 bp, synthetic construct
  misc_feature 1..16: note = Oligo
  source 1..16: mol_type = other DNA
ctgctaacgccgccct

SEQ ID NO: 206 - DNA, 17 bp, synthetic construct
  misc_feature 1..17: note = Oligo
  source 1..17: mol_type = other DNA
cctgtggacgaagtgga

SEQ ID NO: 207 - DNA, 17 bp, synthetic construct
  misc_feature 1..17: note = Oligo
  source 1..17: mol_type = other DNA
atgagagacgtgcgcca

SEQ ID NO: 208 - DNA, 17 bp, synthetic construct
  misc_feature 1..17: note = Oligo
  source 1..17: mol_type = other DNA
tgttatctccgccagcc

SEQ ID NO: 209 - DNA, 17 bp, synthetic construct
  misc_feature 1..17: note = Oligo
  source 1..17: mol_type = other DNA
gtccatgcgtgaccggg

SEQ ID NO: 210 - DNA, 17 bp, synthetic construct
  misc_feature 1..17: note = Oligo
  source 1..17: mol_type = other DNA
tcgggtacgaccgggcc

SEQ ID NO: 211 - DNA, 15 bp, synthetic construct
  misc_feature 1..15: note = Oligo
  source 1..15: mol_type = other DNA
ggaacggactcacac

SEQ ID NO: 212 - DNA, 17 bp, synthetic construct
  misc_feature 1..17: note = Oligo
  source 1..17: mol_type = other DNA
ccatggtgagcaatacg

SEQ ID NO: 213 - DNA, 15 bp, synthetic construct
  misc_feature 1..15: note = Oligo
  source 1..15: mol_type = other DNA
tatccaccgggagct

SEQ ID NO: 214 - DNA, 15 bp, synthetic construct
  misc_feature 1..15: note = Oligo
  source 1..15: mol_type = other DNA
tagtttggccccctt

SEQ ID NO: 215 - DNA, 16 bp, synthetic construct
  misc_feature 1..16: note = Oligo
  source 1..16: mol_type = other DNA
gggccgacccggggag

SEQ ID NO: 216 - DNA, 17 bp, synthetic construct
  misc_feature 1..17: note = Oligo
  source 1..17: mol_type = other DNA
tccgaacgctcgggact

SEQ ID NO: 217 - DNA, 17 bp, synthetic construct
  misc_feature 1..17: note = Oligo
  source 1..17: mol_type = other DNA
actacttcacgagcaag

SEQ ID NO: 218 - DNA, 17 bp, synthetic construct
  misc_feature 1..17: note = Oligo
  source 1..17: mol_type = other DNA
catctccggcgccgctg

SEQ ID NO: 219 - DNA, 17 bp, synthetic construct
  misc_feature 1..17: note = Oligo
  source 1..17: mol_type = other DNA
cccgcgatctggcacgc

SEQ ID NO: 220 - DNA, 15 bp, synthetic construct
  misc_feature 1..15: note = Oligo
  source 1..15: mol_type = other DNA
cccttccagactcct

SEQ ID NO: 221 - DNA, 15 bp, synthetic construct
  misc_feature 1..15: note = Oligo
  source 1..15: mol_type = other DNA
gggcagcgacagcgc

SEQ ID NO: 222 - DNA, 15 bp, synthetic construct
  misc_feature 1..15: note = Oligo
  source 1..15: mol_type = other DNA
cgagagggcggtcgc

SEQ ID NO: 223 - DNA, 15 bp, synthetic construct
  misc_feature 1..15: note = Oligo
  source 1..15: mol_type = other DNA
ccaggggaaccgccc

SEQ ID NO: 224 - DNA, 17 bp, synthetic construct
  misc_feature 1..17: note = Oligo
  source 1..17: mol_type = other DNA
tacagcccccgagatcg

SEQ ID NO: 225 - DNA, 17 bp, synthetic construct
  misc_feature 1..17: note = Oligo
  source 1..17: mol_type = other DNA
tttctccccccgcttcc

SEQ ID NO: 226 - DNA, 17 bp, synthetic construct
  misc_feature 1..17: note = Oligo
  source 1..17: mol_type = other DNA
acccctgggcgaggtgc

SEQ ID NO: 227 - DNA, 17 bp, synthetic construct
  misc_feature 1..17: note = Oligo
  source 1..17: mol_type = other DNA
atacctttcaacccgtc

SEQ ID NO: 228 - DNA, 17 bp, synthetic construct
  misc_feature 1..17: note = Oligo
  source 1..17: mol_type = other DNA
aaatgccacttagaacg

SEQ ID NO: 229 - DNA, 17 bp, synthetic construct
  misc_feature 1..17: note = Oligo
  source 1..17: mol_type = other DNA
tcctgctcgtccgtaga

SEQ ID NO: 230 - DNA, 17 bp, synthetic construct
  misc_feature 1..17: note = Oligo
  source 1..17: mol_type = other DNA
aaacaggcagacgccgg

SEQ ID NO: 231 - DNA, 16 bp, synthetic construct
  misc_feature 1..16: note = Oligo
  source 1..16: mol_type = other DNA
acgcccatatgggagg

SEQ ID NO: 232 - DNA, 15 bp, synthetic construct
  misc_feature 1..15: note = Oligo
  source 1..15: mol_type = other DNA
cgctcgttggcccgc

SEQ ID NO: 233 - DNA, 17 bp, synthetic construct
  misc_feature 1..17: note = Oligo
  source 1..17: mol_type = other DNA
tgggcgactgggcgtga

SEQ ID NO: 234 - DNA, 17 bp, synthetic construct
  misc_feature 1..17: note = Oligo
  source 1..17: mol_type = other DNA
ctggcgaccgccaggta

SEQ ID NO: 235 - DNA, 17 bp, synthetic construct
  misc_feature 1..17: note = Oligo
  source 1..17: mol_type = other DNA
tccccggggggagcgcg

SEQ ID NO: 236 - DNA, 17 bp, synthetic construct
  misc_feature 1..17: note = Oligo
  source 1..17: mol_type = other DNA
ccacgctgcaggcggac

SEQ ID NO: 237 - DNA, 16 bp, synthetic construct
  misc_feature 1..16: note = Oligo
  source 1..16: mol_type = other DNA
gcgagggagatgcagg

SEQ ID NO: 238 - DNA, 17 bp, synthetic construct
  misc_feature 1..17: note = Oligo
  source 1..17: mol_type = other DNA
gacggggtggtcagctt

SEQ ID NO: 239 - DNA, 17 bp, synthetic construct
  misc_feature 1..17: note = Oligo
  source 1..17: mol_type = other DNA
gacccgggcctgggagg

SEQ ID NO: 240 - DNA, 17 bp, synthetic construct
  misc_feature 1..17: note = Oligo
  source 1..17: mol_type = other DNA
ctcgtccggcccctcgc

SEQ ID NO: 241 - DNA, 17 bp, synthetic construct
  misc_feature 1..17: note = Oligo
  source 1..17: mol_type = other DNA
ggctagactgggatagc

SEQ ID NO: 242 - DNA, 17 bp, synthetic construct
  misc_feature 1..17: note = Oligo
  source 1..17: mol_type = other DNA
ggtataccagcgggagg

SEQ ID NO: 243 - DNA, 16 bp, synthetic construct
  misc_feature 1..16: note = Oligo
  source 1..16: mol_type = other DNA
gccctgcaggcaaagg

SEQ ID NO: 244 - DNA, 15 bp, synthetic construct
  misc_feature 1..15: note = Oligo
  source 1..15: mol_type = other DNA
cgtgagcgtttcccg

SEQ ID NO: 245 - DNA, 17 bp, synthetic construct
  misc_feature 1..17: note = Oligo
  source 1..17: mol_type = other DNA
ctgtccgggcggtagcg

SEQ ID NO: 246 - DNA, 17 bp, synthetic construct
  misc_feature 1..17: note = Oligo
  source 1..17: mol_type = other DNA
gctggggccatgtccga

SEQ ID NO: 247 - DNA, 17 bp, synthetic construct
  misc_feature 1..17: note = Oligo
  source 1..17: mol_type = other DNA
cggcgcgcgatgtagtc

SEQ ID NO: 248 - DNA, 17 bp, synthetic construct
  misc_feature 1..17: note = Oligo
  source 1..17: mol_type = other DNA
ggggcccattccacggt

SEQ ID NO: 249 - DNA, 15 bp, synthetic construct
  misc_feature 1..15: note = Oligo
  source 1..15: mol_type = other DNA
gcaacgtccccctac

SEQ ID NO: 250 - DNA, 17 bp, synthetic construct
  misc_feature 1..17: note = Oligo
  source 1..17: mol_type = other DNA
agtaggtcaatcggcag

SEQ ID NO: 251 - DNA, 17 bp, synthetic construct
  misc_feature 1..17: note = Oligo
  source 1..17: mol_type = other DNA
gcacaggcgcgcgcgcc

SEQ ID NO: 252 - DNA, 15 bp, synthetic construct
  misc_feature 1..15: note = Oligo
  source 1..15: mol_type = other DNA
cgccgcgctagttcc

SEQ ID NO: 253 - DNA, 15 bp, synthetic construct
  misc_feature 1..15: note = Oligo
  source 1..15: mol_type = other DNA
gtggccgtcgtacag

SEQ ID NO: 254 - DNA, 16 bp, synthetic construct
  misc_feature 1..16: note = Oligo
  source 1..16: mol_type = other DNA
agcagaggaatccgaa

SEQ ID NO: 255 - DNA, 15 bp, synthetic construct
  misc_feature 1..15: note = Oligo
  source 1..15: mol_type = other DNA
gtcgggttgcccggg

SEQ ID NO: 256 - DNA, 17 bp, synthetic construct
  misc_feature 1..17: note = Oligo
  source 1..17: mol_type = other DNA
ctcctgtccggccaccg

SEQ ID NO: 257 - DNA, 15 bp, synthetic construct
  misc_feature 1..15: note = Oligo
  source 1..15: mol_type = other DNA
ccccgagctccctac

SEQ ID NO: 258 - DNA, 16 bp, synthetic construct
  misc_feature 1..16: note = Oligo
  source 1..16: mol_type = other DNA
gggacagcccgggaaa

SEQ ID NO: 259 - DNA, 16 bp, synthetic construct
  misc_feature 1..16: note = Oligo
  source 1..16: mol_type = other DNA
gctgggagggacaggg

SEQ ID NO: 260 - DNA, 15 bp, synthetic construct
  misc_feature 1..15: note = Oligo
  source 1..15: mol_type = other DNA
tagtcggcccccccg

SEQ ID NO: 261 - DNA, 15 bp, synthetic construct
  misc_feature 1..15: note = Oligo
  source 1..15: mol_type = other DNA
ctctgtcgttccgca

SEQ ID NO: 262 - DNA, 15 bp, synthetic construct
  misc_feature 1..15: note = Oligo
  source 1..15: mol_type = other DNA
tccgccttgcgggcc

SEQ ID NO: 263 - DNA, 15 bp, synthetic construct
  misc_feature 1..15: note = Oligo
  source 1..15: mol_type = other DNA
acgggacgagcacta

SEQ ID NO: 264 - DNA, 15 bp, synthetic construct
  misc_feature 1..15: note = Oligo
  source 1..15: mol_type = other DNA
atgcaccagcgatca

SEQ ID NO: 265 - DNA, 17 bp, synthetic construct
  misc_feature 1..17: note = Oligo
  source 1..17: mol_type = other DNA
gccagctcaaggccctg

SEQ ID NO: 266 - DNA, 16 bp, synthetic construct
  misc_feature 1..16: note = Oligo
  source 1..16: mol_type = other DNA
gcaggcccaccaacag

SEQ ID NO: 267 - DNA, 16 bp, synthetic construct
  misc_feature 1..16: note = Oligo
  source 1..16: mol_type = other DNA
caggagggcggcgtta

SEQ ID NO: 268 - DNA, 17 bp, synthetic construct
  misc_feature 1..17: note = Oligo
  source 1..17: mol_type = other DNA
tcattccacttcgtcca

SEQ ID NO: 269 - DNA, 17 bp, synthetic construct
  misc_feature 1..17: note = Oligo
  source 1..17: mol_type = other DNA
aacatggcgcacgtctc

SEQ ID NO: 270 - DNA, 17 bp, synthetic construct
  misc_feature 1..17: note = Oligo
  source 1..17: mol_type = other DNA
ggacggctggcggagat

SEQ ID NO: 271 - DNA, 17 bp, synthetic construct
  misc_feature 1..17: note = Oligo
  source 1..17: mol_type = other DNA
ccgacccggtcacgcat

SEQ ID NO: 272 - DNA, 17 bp, synthetic construct
  misc_feature 1..17: note = Oligo
  source 1..17: mol_type = other DNA
ttccggcccggtcgtac

SEQ ID NO: 273 - DNA, 15 bp, synthetic construct
  misc_feature 1..15: note = Oligo
  source 1..15: mol_type = other DNA
atgggtgtgagtccg

SEQ ID NO: 274 - DNA, 17 bp, synthetic construct
  misc_feature 1..17: note = Oligo
  source 1..17: mol_type = other DNA
gatacgtattgctcacc

SEQ ID NO: 275 - DNA, 15 bp, synthetic construct
  misc_feature 1..15: note = Oligo
  source 1..15: mol_type = other DNA
actaagctcccggtg

SEQ ID NO: 276 - DNA, 15 bp, synthetic construct
  misc_feature 1..15: note = Oligo
  source 1..15: mol_type = other DNA
gcccaagggggccaa

SEQ ID NO: 277 - DNA, 16 bp, synthetic construct
  misc_feature 1..16: note = Oligo
  source 1..16: mol_type = other DNA
cggactccccgggtcg

SEQ ID NO: 278 - DNA, 17 bp, synthetic construct
  misc_feature 1..17: note = Oligo
  source 1..17: mol_type = other DNA
tagtagtcccgagcgtt

SEQ ID NO: 279 - DNA, 17 bp, synthetic construct
  misc_feature 1..17: note = Oligo
  source 1..17: mol_type = other DNA
gatgcttgctcgtgaag

SEQ ID NO: 280 - DNA, 17 bp, synthetic construct
  misc_feature 1..17: note = Oligo
  source 1..17: mol_type = other DNA
cgggcagcggcgccgga

SEQ ID NO: 281 - DNA, 17 bp, synthetic construct
  misc_feature 1..17: note = Oligo
  source 1..17: mol_type = other DNA
aggggcgtgccagatcg

SEQ ID NO: 282 - DNA, 15 bp, synthetic construct
  misc_feature 1..15: note = Oligo
  source 1..15: mol_type = other DNA
gcccaggagtctgga

SEQ ID NO: 283 - DNA, 15 bp, synthetic construct
  misc_feature 1..15: note = Oligo
  source 1..15: mol_type = other DNA
ctcggcgctgtcgct

SEQ ID NO: 284 - DNA, 15 bp, synthetic construct
  misc_feature 1..15: note = Oligo
  source 1..15: mol_type = other DNA
ctgggcgaccgccct

SEQ ID NO: 285 - DNA, 15 bp, synthetic construct
  misc_feature 1..15: note = Oligo
  source 1..15: mol_type = other DNA
tgtagggcggttccc

SEQ ID NO: 286 - DNA, 17 bp, synthetic construct
  misc_feature 1..17: note = Oligo
  source 1..17: mol_type = other DNA
gaaacgatctcgggggc

SEQ ID NO: 287 - DNA, 17 bp, synthetic construct
  misc_feature 1..17: note = Oligo
  source 1..17: mol_type = other DNA
gggtggaagcgggggga

SEQ ID NO: 288 - DNA, 17 bp, synthetic construct
  misc_feature 1..17: note = Oligo
  source 1..17: mol_type = other DNA
gtatgcacctcgcccag

SEQ ID NO: 289 - DNA, 17 bp, synthetic construct
  misc_feature 1..17: note = Oligo
  source 1..17: mol_type = other DNA
atttgacgggttgaaag

SEQ ID NO: 290 - DNA, 17 bp, synthetic construct
  misc_feature 1..17: note = Oligo
  source 1..17: mol_type = other DNA
aggacgttctaagtggc

SEQ ID NO: 291 - DNA, 17 bp, synthetic construct
  misc_feature 1..17: note = Oligo
  source 1..17: mol_type = other DNA
gttttctacggacgagc

SEQ ID NO: 292 - DNA, 17 bp, synthetic construct
  misc_feature 1..17: note = Oligo
  source 1..17: mol_type = other DNA
gcgtccggcgtctgcct

SEQ ID NO: 293 - DNA, 16 bp, synthetic construct
  misc_feature 1..16: note = Oligo
  source 1..16: mol_type = other DNA
agcgcctcccatatgg

SEQ ID NO: 294 - DNA, 15 bp, synthetic construct
  misc_feature 1..15: note = Oligo
  source 1..15: mol_type = other DNA
cccagcgggccaacg

SEQ ID NO: 295 - DNA, 17 bp, synthetic construct
  misc_feature 1..17: note = Oligo
  source 1..17: mol_type = other DNA
ccagtcacgcccagtcg

SEQ ID NO: 296 - DNA, 17 bp, synthetic construct
  misc_feature 1..17: note = Oligo
  source 1..17: mol_type = other DNA
gggatacctggcggtcg

SEQ ID NO: 297 - DNA, 17 bp, synthetic construct
  misc_feature 1..17: note = Oligo
  source 1..17: mol_type = other DNA
gtggcgcgctccccccg

SEQ ID NO: 298 - DNA, 17 bp, synthetic construct
  misc_feature 1..17: note = Oligo
  source 1..17: mol_type = other DNA
tcgcgtccgcctgcagc

SEQ ID NO: 299 - DNA, 16 bp, synthetic construct
  misc_feature 1..16: note = Oligo
  source 1..16: mol_type = other DNA
cgtccctgcatctccc

SEQ ID NO: 300 - DNA, 17 bp, synthetic construct
  misc_feature 1..17: note = Oligo
  source 1..17: mol_type = other DNA
ggtcaagctgaccaccc

SEQ ID NO: 301 - DNA, 17 bp, synthetic construct
  misc_feature 1..17: note = Oligo
  source 1..17: mol_type = other DNA
cgagcctcccaggcccg

SEQ ID NO: 302 - DNA, 17 bp, synthetic construct
  misc_feature 1..17: note = Oligo
  source 1..17: mol_type = other DNA
agccgcgaggggccgga

SEQ ID NO: 303 - DNA, 17 bp, synthetic construct
  misc_feature 1..17: note = Oligo
  source 1..17: mol_type = other DNA
taccgctatcccagtct

SEQ ID NO: 304 - DNA, 17 bp, synthetic construct
  misc_feature 1..17: note = Oligo
  source 1..17: mol_type = other DNA
gactcctcccgctggta

SEQ ID NO: 305 - DNA, 17 bp, synthetic construct
  misc_feature 1..17: note = Oligo
  source 1..17: mol_type = other DNA
gggccggacgccggaga

SEQ ID NO: 306 - DNA, 16 bp, synthetic construct
  misc_feature 1..16: note = Oligo
  source 1..16: mol_type = other DNA
cacgcctttgcctgca

SEQ ID NO: 307 - DNA, 15 bp, synthetic construct
  misc_feature 1..15: note = Oligo
  source 1..15: mol_type = other DNA
acagcgggaaacgct

SEQ ID NO: 308 - DNA, 17 bp, synthetic construct
  misc_feature 1..17: note = Oligo
  source 1..17: mol_type = other DNA
cagccgctaccgcccgg

SEQ ID NO: 309 - DNA, 17 bp, synthetic construct
  misc_feature 1..17: note = Oligo
  source 1..17: mol_type = other DNA
gccgtcggacatggccc

SEQ ID NO: 310 - DNA, 17 bp, synthetic construct
  misc_feature 1..17: note = Oligo
  source 1..17: mol_type = other DNA
ccccgactacatcgcgc

SEQ ID NO: 311 - DNA, 17 bp, synthetic construct
  misc_feature 1..17: note = Oligo
  source 1..17: mol_type = other DNA
ttgcaccgtggaatggg

SEQ ID NO: 312 - DNA, 15 bp, synthetic construct
  misc_feature 1..15: note = Oligo
  source 1..15: mol_type = other DNA
tactgtagggggacg

SEQ ID NO: 313 - DNA, 17 bp, synthetic construct
  misc_feature 1..17: note = Oligo
  source 1..17: mol_type = other DNA
gtgcctgccgattgacc

SEQ ID NO: 314 - DNA, 17 bp, synthetic construct
  misc_feature 1..17: note = Oligo
  source 1..17: mol_type = other DNA
ggcgggcgcgcgcgcct

SEQ ID NO: 315 - DNA, 15 bp, synthetic construct
  misc_feature 1..15: note = Oligo
  source 1..15: mol_type = other DNA
ccacggaactagcgc

SEQ ID NO: 316 - DNA, 15 bp, synthetic construct
  misc_feature 1..15: note = Oligo
  source 1..15: mol_type = other DNA
tgctctgtacgacgg

SEQ ID NO: 317 - DNA, 16 bp, synthetic construct
  misc_feature 1..16: note = Oligo
  source 1..16: mol_type = other DNA
cgacttcggattcctc

SEQ ID NO: 318 - DNA, 15 bp, synthetic construct
  misc_feature 1..15: note = Oligo
  source 1..15: mol_type = other DNA
ggagcccgggcaacc

SEQ ID NO: 319 - DNA, 17 bp, synthetic construct
  misc_feature 1..17: note = Oligo
  source 1..17: mol_type = other DNA
ggggcggtggccggaca

SEQ ID NO: 320 - DNA, 15 bp, synthetic construct
  misc_feature 1..15: note = Oligo
  source 1..15: mol_type = other DNA
tcccgtagggagctc

SEQ ID NO: 321 - DNA, 16 bp, synthetic construct
  misc_feature 1..16: note = Oligo
  source 1..16: mol_type = other DNA
cagctttcccgggctg

SEQ ID NO: 322 - DNA, 16 bp, synthetic construct
  misc_feature 1..16: note = Oligo
  source 1..16: mol_type = other DNA
actaccctgtccctcc

SEQ ID NO: 323 - DNA, 15 bp, synthetic construct
  misc_feature 1..15: note = Oligo
  source 1..15: mol_type = other DNA
agagcgggggggccg

SEQ ID NO: 324 - DNA, 15 bp, synthetic construct
  misc_feature 1..15: note = Oligo
  source 1..15: mol_type = other DNA
cggatgcggaacgac

SEQ ID NO: 325 - DNA, 15 bp, synthetic construct
  misc_feature 1..15: note = Oligo
  source 1..15: mol_type = other DNA
gcggggcccgcaagg

(19) United States
(12) Patent Application Publication
(10) Pub. No.: US 2025/0283135 A1
(43) Pub. Date: Sep. 11, 2025

(54) ASYMMETRIC ASSEMBLY OF POLYNUCLEOTIDES

(71) Applicant: Ribbon Biolabs GmbH, Vienna (AT)

(72) Inventor: Harold Paul VLADAR, Vienna (AT)

(21) Appl. No.: 18/853,050

(22) PCT Filed: Mar. 31, 2023
(86) PCT No.: PCT/EP2023/058508

§ 371 Date: Sep. 30, 2024

(30) Foreign Application Priority Data
Mar. 31, 2022 (EP) 22165859.4

(51) Int. Cl. C12P 19/34; C07H 21/00

(52) U.S. Cl. CPC C12P 19/34; C07H 21/00

(57) ABSTRACT

A method of assembling nucleic acid (NA) building blocks to produce a double stranded (ds) target polynucleotide, comprising assembling NA building blocks according to a workflow comprising multiple assembly tiers, wherein, in a hierarchical assembly, at least one asymmetric pair of NA building blocks of different length that originate from different assembly tiers, is connected to produce an intermediate polynucleotide in at least one of said multiple assembly tiers, wherein said intermediate polynucleotide is used as a building block to connect further NA building blocks at both ends of said intermediate polynucleotide in a further assembly tier.

### FIELD OF THE INVENTION

The invention relates to a novel method for synthesizing a double stranded (ds) polynucleotide having a predefined sequence using a workflow of multiple assembly tiers including at least one asymmetric assembly of nucleic acid (NA) building blocks.

### BACKGROUND OF THE INVENTION

Some methods for the synthesis and assembly of nucleic acids implement a hierarchical assembly approach where multiple pairs of DNA, RNA, TNA or XNA oligonucleotides, single or double stranded, are ligated in parallel and independently in order to obtain a first set of intermediate products that are longer.

WO2019073072A1 discloses a hierarchical assembly approach, wherein, after completing these first steps, the first intermediate products are again combined in pairs, thereby obtaining even longer polynucleotides. Further steps proceed in the same fashion until a long target polynucleotide is obtained (FIG. 1).

This and other methods rely on matching overhangs between any two starting oligonucleotides (oligos) or intermediate products and are thus subject to ambiguities either because overhangs other than the intended ones can ligate, resulting in a wrong intermediate product or “mis-assembly”. These assemblies can be represented as graphs where the vertices indicate transfer/pooling steps, and the nodes represent the DNA oligos or intermediate polynucleotides. When the specific workflow is disclosed, the graph is not attributed to specific experimental steps or reaction or to any particular method but is used only to indicate a visual aid to imply the hierarchical nature. Prior art hierarchical assemblies use a symmetrical workflow, a sequential addition of oligos or pooled reactions, and the notion of “hierarchical” is used to denote a tiered, but generally sequence-independent approach. This is understood as a workflow where all oligos react to obtain reaction products that are longer than the components of the reaction products, which is repeated in several steps.

Although a symmetric hierarchical assembly method is improved over methods using pooled or sequential workflows, it does not in its own prevent mis-assemblies, if there is a variety of constituting building blocks (oligo or polynucleotides) which have a region of sufficient sequence overlap, that could result in an erroneous assembly (see e.g., FIGS. 2,3).

Although the last few years have seen considerable progress in the techniques for synthesizing DNA, there are still severe restrictions in terms of volume, throughput and, specially, length of DNA.

WO2021055962A1 discloses compositions and methods for template-free double stranded geometric enzymatic nucleic acid synthesis of arbitrarily programmed nucleic acid sequences.

WO2018203056A1 discloses a nucleic acid for use in DNA assembly, wherein the nucleic acid comprises at least one methylation-protectable restriction element, the methylation-protectable restriction element comprising: a restriction enzyme recognition sequence that is recognized by a restriction enzyme that cleaves outside of the recognition sequence; and a DNA methylase recognition sequence, wherein the restriction enzyme recognition sequence and the DNA methylase recognition sequence overlap such that the base modified by the DNA methylase lies within the restriction enzyme recognition sequence, wherein the DNA methylase recognition sequence is not identical to or enclosed by the restriction enzyme recognition sequence, and wherein the DNA methylase recognition sequence does not overlap with the sequence that would form the overhang end sequence generated by the restriction enzyme.

### SUMMARY OF THE INVENTION

It is an objective of the present invention to provide an improved method for synthesizing double stranded (ds) polynucleotides, aiming to reduce the number of mis-assemblies when synthesizing a target ds polynucleotide. It is a further objective of the present invention to provide an improved method for synthesizing ds polynucleotides where a variety of building blocks (oligo or polynucleotides) comprising the same matching region of sequence overlap are used.

The object is solved by the subject of the present invention.

According to the invention, there is provided a method of assembling nucleic acid (NA) building blocks to produce a double stranded (ds) target polynucleotide, comprising assembling NA building blocks according to a workflow comprising multiple assembly tiers, wherein at least one asymmetric pair of NA building blocks which are of different length, preferably at least one asymmetric pair of ds NA building blocks, is connected to produce an intermediate polynucleotide in at least one of said multiple assembly tiers, wherein said intermediate polynucleotide is used as a building block to connect further NA building blocks at both ends of said intermediate polynucleotide in one or more further assembly tiers.

According to a specific aspect, there is provided a method of assembling nucleic acid (NA) building blocks to produce a double stranded (ds) target polynucleotide, comprising assembling NA building blocks according to a workflow comprising multiple assembly tiers, wherein, in a hierarchical assembly, at least one asymmetric pair of NA building blocks of different length that originate from different assembly tiers is connected to produce an intermediate polynucleotide in at least one of said multiple assembly tiers, wherein said intermediate polynucleotide is used as a building block to connect further NA building blocks at both ends of said intermediate polynucleotide in one or more further assembly tiers.

Specifically, the workflow is an asymmetric assembly workflow. Specifically, the assembly comprises at least one asymmetric assembly assembly of NA building blocks.

Specifically, the workflow is a hierarchical asymmetric workflow. Specifically, the hierarchical assembly comprises at least one asymmetric assembly of NA building blocks.

Specifically said workflow comprises at least one tier, which comprises at least one asymmetric assembly of NA building blocks.

Specifically said workflow comprises at least one tier, in which at least one asymmetric pair of NA building blocks of different length is assembled. Specifically, said at least one asymmetric pair of NA building blocks of different length is assembled by an asymmetric assembly.

Specifically, the asymmetric assembly is non-geometric.

Specifically, said hierarchical assembly comprises at least one asymmetric assembly of said at least one asymmetric pair of NA building blocks.

Specifically, a pair of NA building blocks consists of two NA building blocks with matching sequences to allow an assembly of the two NA building blocks.

Specifically, a pair of ds NA building blocks, such that the pair of ds NA building blocks is assembled by connecting their ends such as by hybridizing the 5′-end of one NA building block of said pair to the 3′-end of the other one of said pair, to produce an assembled ds polynucleotide or nucleic acid molecule comprising a site of assembly, which is where the matching sequences have hybridized.

Specifically, said asymmetric pair of NA building blocks is a pair of one ds NA building block and one ss NA building block, or a pair of two ds NA building blocks.

Specifically, a pair of ds NA building blocks which is an asymmetric pair of ds NA building blocks consists of two ds NA building blocks of different length. In contrast, in a symmetric pair of ds NA building blocks, both NA building blocks have the same length.

Specifically, the length of an asymmetric pair of NA building blocks may differ such that at least one of the NA building blocks has a shorter length than the other NA building block of said asymmetric pair of ds NA building blocks e.g., at least any one of 10%, 20%, 30%, 40%, 50%, 60%, 70%, 80%, 90%, or 95%, up to 99% shorter.

Specifically, the asymmetric pair of ds NA building blocks is a pair of ds NA building blocks, and the difference in length is determined comparing the double stranded part of the ds NA building blocks.

Specifically, where a pair of two of NA building blocks of different length is assembled, the present disclosure provides for the following embodiments:
a) both NA building blocks are de novo synthesized and used as a starting NA building block in a first assembly tier; or
b) one of the NA building blocks is produced in a previous assembly tier, and the other one is de novo synthesized as a starting NA building block; or
c) both of the NA building blocks are produced by previous assembly tiers, optionally wherein the number of assembly tiers to produce one of the NA building blocks differs from the number of assembly tiers to produce the other one of the NA building blocks.

According to a specific aspect, one NA building block of said at least one asymmetric pair is a starting NA building block, and said asymmetric pair is assembled in a second or further tier of assembly.

Specifically, one NA building block of said at least one asymmetric pair is a starting NA building block, in particular a ss or ds NA building block, such as a ss or ds oligo, and the other one of the asymmetric pair is an NA building block which is a reaction product obtained by assembly of NA building blocks in a first, second or further assembly tier.

By assembling an asymmetric pair of ds NA building blocks, a site of assembly can be chosen that is not in the center of the assembly product, but within a different region that avoids mis-assemblies.

Specifically, one of the NA building blocks of the asymmetric pair is a reaction product obtained by assembly of NA building blocks in a previous assembly tier, and the other one of the asymmetric pair is a starting NA building block, or a reaction product obtained by assembly of NA building blocks in an assembly tier that is different from said previous assembly tier.

Specifically, in an assembly tear, one or more pairs of NA building blocks are assembled. The number of assembly tiers, reaction steps and corresponding reaction products is basically determined by the length of the target polynucleotide. In order to synthesize large target polynucleotides, a series of assembly tiers may be needed for assembly into the target polynucleotide e.g., at least 5, 10, 20, 50, 100, 500, 1.000, 5.000 or more may be necessary. Each tier comprises a series of parallel assembly reactions. Typically, the number of assembly reactions decreases with increasing length of the NA building blocks in a tier.

Specifically, an intermediate polynucleotide as used herein is a ds NA building block which is a product of assembling a pair of NA building blocks. Specifically, the intermediate polynucleotide is used as a building block to connect further NA building blocks at both ends of said intermediate polynucleotide, such as to assembly at least one NA building block in both directions, in one or more further assembly tiers. Assembling of NA building blocks in both directions results in an extension of the 3′-end and an extension of the 5′-end of said intermediate polynucleotide. Such extensions can be done in parallel and/or in the same assembly tier, or consecutively.

Specifically, the intermediate polynucleotide is assembled with further NA building blocks at its 5′-end and its 3′-end (i.e., at both of its ends) in the same assembly tier, optionally wherein assembly at both ends is carried out simultaneously or consecutively.

Specifically, the intermediate polynucleotide is assembled with further NA building blocks at its 5′-end and its 3′-end (i.e., at both of its ends) in the different assembly tiers, optionally wherein assembly of one of the ends of the intermediate polynucleotide with a NA building block is carried out in one assembly tier, and assembly of the other end of the intermediate polynucleotide with another NA building block is carried out in another assembly tier.

In a specific embodiment, the intermediate polynucleotide is assembled with a NA building block at its 5′-end in a further assembly tier, thereby obtaining a reaction product, which reaction product is assembled with another NA building block at its 3′-end in the same or another further assembly tier.

In any case, assembly of the intermediate polynucleotide with further NA building blocks at both ends can be carried out simultaneously or consecutively.

Where the intermediate polynucleotide is assembled with further NA building blocks at both ends, such polynucleotide is considered as an intermediate that is different from a prefinal polynucleotide.

Typically, in a final step of assembly, a prefinal polynucleotide is used that has been produced in a prefinal assembly step, which is then assembled with a final NA building block in the final step of assembly. Optionally, a final NA building block is used which comprises a blunt end, or an end that is designed to match a site of a nucleotide construct (such as a vector or plasmid) to incorporate the target ds polynucleotide in said nucleotide construct. In such final step of assembly, the prefinal polynucleotide is assembled with another NA building block only at one of its ends.

Specifically, the NA building blocks are oligonucleotides or polynucleotides e.g., fragments of the ds target polynucleotide.

Specifically, NA building blocks are either double stranded such as ds oligos or ds polynucleotides, in particular ds intermediate polynucleotides or prefinal polynucleotides, or single stranded such as ss oligos or ss polynucleotides.

In a specific aspect of the method described herein, pairs of matching ss oligos are assembled to produce ds NA building blocks. Preferably, said pairs of matching ss oligos are transferred into separate reaction containments to produce the respective ds NA building blocks.

Specifically, the NA building blocks have a length ranging from 8 nucleotides up to 20% of the length of the target polynucleotide.

In a specific embodiment, ss NA building blocks are ss oligos or ss polynucleotides with a length of at least 8, 9, 10, 11, 12, 13, 14, 15, 16, 17, 18, 19, 20, 21, 22, 23, 24, 25, 26, or even more nucleotides. Specifically, ss NA building blocks can have a length of at least 30, 40, 50, 60, 70, 80, 90, or 100 nucleotides. In specific cases, the length of ss NA building blocks is limited, and may have a length of less than 900, 800, 700, 600, 500, 400, 300, 200 or 100 nucleotides. Specifically, ss NA building blocks may have a length of up to 26, 25, 24, 23, 22, 21, 20, 19, 18, 17 or 16 nucleotides.

In a specific embodiment, ds NA building blocks are ds oligos or ds intermediate or prefinal polynucleotides, which comprise a double stranded part that has a length of at least 4, 5, 6, 7, 8, 9, 10, 11, 12, 13, 14, 15, 16, 17, 18, 19, 20, 21, 22, 23, 24, 25, 26, or even more base pairs. Specifically, the double stranded part of the ds NA building blocks can have a length of at least 30, 40, 50, 60, 70, 80, 90, or 100 base pairs. In specific cases, the length of the ds NA building blocks is limited, such as ds NA building blocks with a length of the double stranded part of less than 900, 800, 700, 600, 500, 400, 300, 200 or 100 base pairs. Specifically, the double stranded part of said ds NA building blocks have a length of up to 26, 25, 24, 23, 22, 21, 20, 19, 18, 17 or 16 base pairs.

Specifically, the NA building blocks can have a length of up to any one of 20%, 15%, or 10% of the target nucleotide. Specifically, any building blocks that have a length of at least any one of 5%, 6%, 7%, 8%, 9%, or 10% of the target nucleotide are ds.

Specifically, a ds NA building block has one or two overhangs, which is at one or both of its ends, in particular at the 5′-end and/or at the 3′-end of the ds NA building block.

Specifically, a ds NA building block comprising overhangs on both ends and no blunt end may be used as an intermediate polynucleotide.

Specifically, NA building blocks are assembled according to a defined workflow.

Specifically, the workflow may provide for the production of ds NA building blocks from respective single stranded (ss) NA building blocks, such as by assembly of ss oligos. Specifically, ds NA building blocks can be produced by assembly of ss NA building blocks before their use in a method to assemble larger polynucleotides.

Specifically, the workflow may provide for the production of ds NA building blocks from respective ds NA building blocks, such as by assembly of ds oligos. Specifically, ds NA building blocks can be produced by assembly of ds NA building blocks before their use in a method to assemble larger polynucleotides.

According to a specific aspect described herein, an asymmetric hierarchical workflow is employed.

Specifically, said workflow is determined using an algorithm.

Specifically, said algorithm selects pairs of matching NA building blocks and optionally ss NA building block linkers (such as ss oligo linkers), if necessary, and determines the assembly workflow, not by a mere sequence partitioning, but by determining an optimal or near-optimal way to assemble the target ds polynucleotide, avoiding mismatches or undesired reaction products as far as possible.

NA building blocks and assembly workflow are specifically selected to avoid undesired (incorrect) reactions or reaction products, such as palindromic sequences, runaway reactions and unambiguous assembly.

Specifically, the assembly workflow is automated.

Specifically, the automated workflow employs microfluidic handlers that are capable of transferring serially or in parallel the full or partial contents of one or several compartments into other pre-specified compartments that may or may not be empty.

In certain embodiments of the method described herein, purification of the reaction products may be necessary at one more assembly step. If there are incorrect reaction products besides the correct reaction products, such incorrect reaction products may be suitably separated from the correct ones e.g., as follows: using gel electrophoresis to detect oligonucleotides or polynucleotides of a certain size and excising and purifying bands of the gel corresponding to the size of the desired reaction product. Specifically, correct reaction products can be detected by incorporation of tags or labels into the sequence. Specifically, oligos or polynucleotides may be captured using biotinylated oligonucleotide adapters capable of hybridizing with an overhang of a NA building block wherein, said adapters are fixed to the substrate and coated with streptavidin. Non-captured incorrect products are eliminated by washing and subsequently, the correct products are released from the adapters by increasing the temperature. Specifically, further separation methods well-known in the art may be applied. Specifically, such methods may involve chromatographic or affinity separation methods.

According to the method provided herein, NA building blocks are assembled to intermediate polynucleotides and ultimately the target polynucleotide.

Specifically, said assembly is by connecting matching sequences.

Specifically, said assembly is by connecting a matching ss NA building block to another ss NA building block and/or to an overhang of a ds NA building block; and/or by connecting matching overhangs of a pair of ds NA building blocks.

Specifically, matching NA building blocks or respective parts of the NA building blocks such as overhangs comprise or consist of ss nucleotide sequences which are sufficiently complementary to each other, to allow hybridizing said nucleotide sequences.

Specifically, assembly is by hybridizing complementary nucleotide sequences (or sufficiently complementary sequences).

According to a specific aspect, assembly may also include connecting NA building blocks by a ligation reaction which comprises enzymatic, chemical, or an adaptor ligation.

Specifically, said ligation reaction is an enzymatic ligation reaction using a ligase, preferably a ligase, such as any one of a T3, T4 or T7 DNA ligase, or a RNA ligase, a polymerase or ribozymes. Preferably T4 DNA ligase, T7 DNA Ligase, T3 DNA Ligase, Taq DNA Ligase, DNA polymerase, or engineered enzymes are used in the ligation reaction. Preferably, the following ligation reaction is used: T4 DNA Ligase, at a concentration of 10 cohesive end units per L supplemented with 1 mM ATP (Sambrook and Russel, 2014, Chapter 1, Protocol 17).

Specifically, said assembly is directly by hybridizing matching overhangs of a ds NA building block, or indirectly by hybridizing a suitable ss NA building block linker (i.e., a ss NA linker), which ss NA linker can be a ss oligo or ss polynucleotide, optionally contained in and obtained from a respective library.

In a specific embodiment of the method described herein, a solid carrier is used to immobilize one or more of said NA building blocks, the target polynucleotide, or one or more intermediate(s) of assembly. Specifically, said nucleic acids may be immobilized on a solid phase using a tag, for example a biotin tag.

Specifically, the NA building blocks are provided in separate containments e.g., a library of NA building blocks. A library of NA building blocks is conveniently used as a source of NA building blocks, which can be selected from the library on demand, such as to allow assembling the selected NA building blocks according to a pre-defined workflow.

Specifically, ss and ds NA building blocks can be provided in separate containments of a library, which can be readily used as a source of NA building blocks for the purpose described herein. In a specific embodiment, ds NA building blocks are prepared in and sourced from the same containments.

In a specific embodiment of the method described herein, said NA building blocks are obtained from a library of NA building blocks, which comprises a diversity of NA building blocks, each contained in a separate library containment.

Specifically, said library comprises ss NA building blocks, preferably wherein said ss NA building blocks have been obtained by de novo synthesis, such as by chemical or enzymatic means.

Specifically, said library comprises ds NA building blocks, preferably produced from respective ss NA building blocks from the library.

The library described herein specifically comprises or consists of library members which are ss and/or ds NA building blocks, preferably the library comprises or consists of both of ss and ds NA building blocks. Preferably, library members are pre-built, optionally provided in a storage stable form, and located at defined positions within an array device. Library members can be synthesized and stored in the array device until needed.

Specifically, the NA building blocks can be produced by any suitable prior art method, such as chemical polynucleotide (or oligonucleotide) synthesis methods, including the H-phosphonate, phosphodiester, phosphotriester or phosphite triester synthesis methods, or any of the massively parallel oligonucleotide synthesis methods e.g., microarray or microfluidics-based oligonucleotide synthesis e.g., as described in Gao et al. 2001, LeProust et al. 2010, or Bonde et al. 2014.

Specifically, the NA building blocks can be produced by any of the enzymatic polynucleotide (or oligonucleotide) synthesis methods e.g., ssDNA synthesis by DNA polymerase proteins or by reverse transcriptase proteins, which produce hybrid RNA-ssDNA molecules. Specifically, the enzymatic polynucleotide synthesis reaction is performed in vitro.

Specific embodiments refer to NA building blocks which are modified by any one or more of phosphorylation, methylation, biotinylation, or linkage to a fluorophore or quencher. Therefore, the library described herein may comprise library members which are NA building blocks that can be any or all of the following: unmodified ss; phosphorylated ss; methylated ss; biotinylated ss; phosphorylated, biotinylated and methylated ss; unmodified ds; phosphorylated ds; methylated ds; biotinylated and phosphorylated ds; biotinylated and methylated ds. Preferably, library members comprise a 5′-phosphorylation. Specifically, the library described herein comprises ss NA building blocks comprising fluorophores or quenchers and ds NA building blocks comprising fluorophores or quenchers.

Specifically, said NA building blocks are provided in a storage-stable form, preferably a form which is storage-stable for at least 6 months at room-temperature.

Specifically, the library of NA building blocks described herein is storage stable, comprising the NA building blocks in storage containments.

Specifically, the library may comprise dried NA building blocks. NA building blocks can be stored in storage containments in a dry state. Dry-state is, for example, achieved by lyophilization, freeze drying, evaporation, crystallization or the like. The enzymes which catalyze the degradation of nucleic acids are typically active at room temperature in a fluid biomolecule preparation. Dry-state storage inhibits such enzymatic activity because such enzymes are generally inactive upon de-hydration and because the degradative chemical reactions which they catalyze typically entail the addition of water (i.e., hydrolysis) of a protein or nucleic acid molecule, thus producing protein or nucleic acid backbone cleavage. In the dry state, there is little or no water (e.g., less than 5%, 4%, 3%, 2% or 1% (w/w) water) as a chemical reactant to support such enzyme catalysis. Additionally, any non-enzymatic hydrolysis of protein or nucleic acid is similarly inhibited, since water is generally unavailable for such reactions.

In a specific embodiment, the ds target polynucleotide produced according to the method described herein, is provided and/or stored in a dry state.

Specifically, a reaction containment or a storage containment is a compartment unit, such as a well, of any one of a microtiter plate, a microfluidic microplate, a set of capillaries, a microarray or a biochip, preferably a DNA and/or RNA biochip.

Specifically, for hybridization of matching nucleic acid molecules, said matching nucleic acid molecules are placed in a reaction containment that features an environment in which one nucleic acid strand bonds to a second nucleic acid strand by complementary strand interactions and hydrogen bonding to produce a double stranded oligonucleotide or polynucleotide. Such conditions include a mixture of the matching nucleic acid molecules as reaction components in an aqueous or organic solution, further components (e.g., salts, chelating agents, formamide) and their concentrations in said solution, and the temperature of the mixture. Other well-known factors, such as the length of incubation time or reaction chamber dimensions may contribute to the environment.

Specifically, the NA building blocks described herein are produced by synthesizing the oligonucleotide or polynucleotide sequence from nucleotide building blocks by any of the polynucleotide synthesis methods, wherein the building blocks are comprised of “A” denoting deoxyadenosine, “T” denoting deoxythymidine, “G” denoting deoxyguanosine, or “C” denoting deoxycytidine or other natural nucleosides (e.g. adenosine, thymidine, guanosine, cytidine, uridine), nucleotide-analogs e.g., inosine and 2′-deoxyinosine and theirs derivatives (e.g. 7′-deaza-2′-deoxyinosine, 2′-deaza-2′-deoxyinosine), azole- (e.g. benzimidazole, indole, 5-fluoroindole) or nitroazole analogues (e.g. 3-nitropyrrol, 5-nitroindol, 5-nitroimidazole, 4-nitropyrazole, 4-nitrobenzimidazole) and their derivatives, acyclic sugar analogues (e.g. those derived from hypoxanthine- or indazole derivatives, 3-nitroimidazole, or imidazole-4,5-dicarboxamide), 5′-triphosphates of universal base analogues (e.g. derived from indole derivatives), isocarbostyril and its derivatives (e.g. methylisocarbostyril, 7-propynylisocarbostyril), hydrogen bonding universal base analogues (e.g. pyrrolopyrimidine), or any of the other chemically modified bases (such as diaminopurine, 5-methylcytosine, isoguanine, 5-methyl-isocytosine, K-2′-deoxyribose, P-2′-deoxyribose). Likewise, the NA building blocks may comprise “U” denoting uracil, a deoxyuridine, or deoxyuridine site. An enzyme Uracil DNA glycosylase can be used to transform the deoxyuridine site into an abasic site, which subsequently can be cut using glyeosylate-lyase endonuclease VIII, thereby releasing the DNA.

The building blocks are linked by phosphodiester linkage or peptidyl linkages or by phosphorothioate linkages or by any of the other types of nucleotide linkages.

Specifically, the target polynucleotide has a length of at least 132 base pairs (bps). Specifically, said target polynucleotide has a length of at least 150, 200, 250, 300, 400, 500, 600, 700, 800, 900, 1.000 bps.

Specifically, by the method described herein target polynucleotides with a length up to 1.000, 5.000, 10.000 or 100.000 bps or even longer can be produced, at a low price and at a high speed.

Specifically, the nucleotide sequence of a target ds polynucleotide or respective template can be of one of natural or artificial origin.

Specifically, the target ds polynucleotide has blunt ends on both ends.

According to a specific aspect, a blunt end of the target ds polynucleotide can be produced by using a pair of NA building blocks wherein one of the NA building blocks comprises a blunt end, thereby obtaining a terminal NA building block with a blunt end. According to another specific aspect, a ss NA building block and a ds NA building block are assembled, wherein the ss NA building block is complementary to an overhang of the ds NA building block, such as to hybridize the complementary sequences without generating any further overhang, thereby producing a blunt end.

Specifically, the method provided herein comprises a finalization step.

Specifically, said finalization step serves to add one or more nucleotide(s) which correspond to those previously removed from the 3′-end and 5′-end, respectively, to prepare a template of such target ds polynucleotide for the purpose of assembly of the target ds polynucleotide according to a template sequence, such as e.g., to generate blunt ends. Specifically, one or more NA building blocks may be selected for producing blunt ends, which are complementary to any overhang of a prefinal intermediate polynucleotide i.e., complementary to the sticky ends of the polynucleotide. Specifically, respective oligos can be used as primers in a PCR reaction to amplify the final product and to add the remaining NA building blocks to each strand to synthesize the complete target polynucleotide with blunt ends.

Specifically, said finalization step comprises a purification step of a PCR product that has been produced employing standard kits, such as the Monarch PCR & DNA clean up kit from New England Biolabs (product no. T1030), to eliminate remaining NA building blocks, oligos, enzymes and reagents, thereby obtaining the target ds polynucleotide as a purified DNA product, ready for further use.

According to a specific aspect, the method described herein further comprises a step of enriching the target polynucleotide or one or more intermediates of assembly, by polymerase chain reaction (PCR).

Specifically, the target polynucleotide or one or more intermediates of assembly may be purified by immobilization on a solid phase using a tag, for example a biotin tag, and enrichment using, e.g., PCR amplification. According to a preferred embodiment, two sets of primers are used for target specific enrichment and simultaneous elimination of the tag. Specifically, by using a set of primers specific to the 5′ end of the leading strand and a set of primers specific to the 5′ end of the lagging strand of the polynucleotide that is to be enriched, each comprising a primer that is complementary to at least the overhang and a primer that is complementary to the core sequence of the polynucleotide, the target polynucleotide is amplified without the tag sequence. This has the profound advantage that no additional step is required to remove the tag sequence, e.g., by enzymatic digestion.

The degree of purification is understood as the amount of NA building block copies per volume or per total (poly) nucleotide mass. Various methods to determine the degree of purity of a preparation of nucleic acid molecules are known to a person skilled in the art. Specifically, the degree of purity may be determined using gel electrophoresis, next generation sequencing or qPCR.

In a specific embodiment of the invention, said target ds polynucleotide is sequenced to verify the degree of identity with the sequence of a template or a sequence of interest (SOI). Any suitable sequencing method may be used, for example any one of SNP genotyping methods, including hybridization-based methods (e.g. molecular beacons, SNP microarrays, restriction fragment length polymorphism, PCR-based methods, including Allele-specific PCR, primer extension-, 5′-nuclease or Oligonucleotide Ligation Assay, Single strand conformation polymorphism, Temperature gradient gel electrophoresis, Denaturing high performance liquid chromatography, High-resolution Melting of the entire amplicon (HRM), SNPlex and surveyor nuclease assay; Sequencing based mutation analysis, including capillary sequencing or high-throughput sequencing of an entire PCR amplicon of the PTR (amplicon sequencing). Such high-throughput (HT) amplicon sequencing methods include, but are not restricted to polony sequencing, pyrosequencing, Illumina (Solexa) sequencing, SOLID sequencing, semiconductor sequencing, DNA nanoball sequencing, Heliscope single molecule sequencing, Single molecule real time (SMRT) sequencing, Nanopore DNA sequencing, tunnelling currents DNA sequencing, sequencing by hybridization, sequencing with mass spectrometry, Microfluidic Sanger sequencing, Microscopy-based sequencing, RNAP sequencing.

In a specific embodiment, the method described herein further comprises a step of processing the target polynucleotide or intermediates of assembly, by means of enzymatic modification employing restriction enzymes or kinases, or by chemical means, to facilitate further ligation, or to facilitate cloning the target polynucleotide into a vector or plasmid.

In a further specific embodiment of the invention, the target ds polynucleotide can be further modified to produce a derivative thereof, which is any of a ds DNA, ss DNA or RNA molecule, e.g. comprised in a vector, such as a plasmid.

Specifically, said target ds polynucleotide can be modified by enzymatic modification, employing any one or more of methyltransferases, kinases, CRISPR/Cas9, multiplex automated genome engineering (MAGE) using A-red recombination, conjugative assembly genome engineering (CAGE), the Argonaute protein family (Ago) or a derivative thereof, zinc-finger nucleases (ZFNs), transcription activator-like effector nucleases (TALENs), meganucleases, tyrosine/serine site-specific recombinases (Tyr/Ser SSRs), hybridizing molecules, sulfurylases, recombinases, nucleases, DNA polymerases, RNA polymerases or TNases.

### FIGURES

FIG. 1. An example of a symmetric assembly tree with the corresponding tiers indicated.

FIG. 2. An example of an asymmetric assembly tree where two components (c and d) would fail to assemble, causing all the connected nodes to fail as well.

FIG. 3. (a) Double stranded oligonucleotides with sticky ends used as assembly reagents (Sequence IDs 18-25). (b-c) Alternative assembly trees for the four reagents.

FIG. 4. (a) Method for calculating the Adjacency Matrix for two ds oligonucleotides with given overhangs. (b-c) Examples of the 4×4 adjacency matrices with minor modifications to the overhangs.

FIG. 5. (a) An example of a symmetric assembly tree with the corresponding scores indicated for every node. (b) The optimised version of the assembly tree where the asymmetric structure resolves the problematic nodes and gives an overall acceptable score. (c) The two Ligation Matrices used to calculate the scores. The non-zero elements are the ones corresponding to overhangs with matching sequences, and the gray areas are the ones that will cause misligations.

FIG. 6. The introduction of “zeromers” in the automated assembly setup allows the use of repetitive, symmetric movements of a liquid handler arm to assemble a molecule following an asymmetric tree.

FIG. 7. (a) The layout of reagents in a plate used to assemble a molecule following a symmetric tree. (b) The layout of reagents in a plate used to assemble a molecule following an asymmetric tree.

FIG. 8. Nucleotide sequences referred to herein.

FIG. 9. Misligation matrices for symmetric and asymmetric assembly trees, used to calculate the scores.

FIG. 10. (a) The symmetric assembly of a 256 bp molecule shows two problematic nodes that cause the assembly to fail. (b) Optimized asymmetric assembly solves the issues caused by the misligations and enables the assembly.

FIG. 11. Electropherograms of the final assembly products of symmetric (continuous line) and asymmetric (dashed line) assemblies. The correct size molecule (256 bp) is only visible in the asymmetric case.

FIG. 12. Three examples of assembly trees/partitions for the same SOI, showing how the score is maximized by many folds when the adaptive partitioning method is combined with the optimization of the asymmetric assembly tree.

FIG. 13 shows a graphical representation of the product of the ligation and misligation matrix (Example 5).

FIG. 14 shows a list of oligo sequences resulting from partitioning a SOI (Example 5).

FIG. 15 shows a list of oligo sequences resulting from adaptive partitioning a SOI (Example 5).

FIG. 16: graphical representation of assembly trees: A) Fully symmetric tree. At each tier, reaction products from the same previous tier are used to have further reaction products. The “leafs” of the tree are the starting oligos. T=Tiers. R.P.=Reaction products. Note that at every step the number of reaction product decreases, in particular, they halve at every step; B) Asymmetric tree. The reactions may involve products (double dashed arrows) or starting oligos (double arrows) that are from any other previous tier (not all instances indicated). The “leafs” of the tree are the starting oligos. T=Tiers. R.P.=Reaction products. Note that at any step the number of reaction product may increase and do not halve at every step.

### DETAILED DESCRIPTION

Specific terms as used throughout the specification have the following meaning.

As used herein, the terms “a”, “an” and “the” are used herein to refer to one or more than one i.e., to at least one. The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “5′-end” or “3′-end” of a NA building block is herein understood to refer to the side or orientation of an end of the NA building block, and specifically understood in the following way.

The 5′-end of a ss NA building block is understood to comprise or consist of the 5′-terminal region e.g., a part of the ss oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ss molecule, which 5′-terminal region is including the 5′-terminus, i.e. the 5′-terminal nucleotide.

The 3′-end of a ss NA building block is understood to comprise or consist of the 3′-terminal region e.g., a part of the ss oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ss molecule, which 3′-terminal region is including the 3′-terminus, i.e. the 3′-terminal nucleotide.

The 5′-end of a ds NA building block is understood to comprise or consist of the 5′-terminal region of the leading strand e.g., a part of the leading strand of the ds oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ds molecule, which 5′-terminal region is including the 5′-terminus, i.e. the 5′-terminal nucleotide.

The 3′-end of a ds NA building block is understood to comprise or consist of the 3′-terminal region e.g., a part of the leading strand of the ds oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ds molecule, which 3′-terminal region is including the 3′-terminus, i.e. the 3′-terminal nucleotide.

Typically, assembly of the NA building blocks as described herein is by end-to-end ligating or connecting (e.g., annealing, hybridizing or linking), wherein a 5′-end of a NA building block is connected to the 3′-end of another NA building block. NA building blocks can be ss or ds. A ss NA building block can be connected to another ss NA building block, or a ds NA building block, and vice versa. A ds NA building block can be connected to another ds building block. The assembly products will comprise both assembled NA building blocks.

Single-strand annealing of complementary sequences may lead to ds NA building blocks with overhangs or blunt ends. Assembly of a pair of ss NA building blocks typically results in a ds NA building block, wherein one ss NA building block is the leading strand and the other one is the lagging strand. In such case, assembly is not an end-to-end assembly, but a side-to-side assembly to a ds molecule, wherein the 5′-end and 3-end of the ss NA building block which is comprised as a leading strand defines the 5′-end and 3′-end of the ds molecule, respectively.

The term “algorithm” as used herein refers to a self-contained sequence of actions to be performed. An algorithm is an effective method that can be expressed within a finite amount of space and time and in a well-defined formal language for calculating a function. Starting from an initial state and initial input the instructions describe a computation that, when executed, proceeds through a finite number of well-defined successive states, eventually producing “output” and terminating at a final ending state. The transition from one state to the next is necessarily deterministic.

The term “assembly” or “assemble” refers to the formation of an oligonucleotide or polynucleotide by a ligation reaction e.g., linking and/or hybridizing single stranded and/or double stranded NA building blocks. Specifically, said assembly is by any method of annealing or hybridizing nucleotide sequences which are complementary to each other, or comprise regions that are complementary or at least partially complementary to each other.

Assembly of the target ds polynucleotide can either be directly by hybridizing matching ss NA building blocks, overhangs of ds NA building blocks, or indirectly by hybridizing one or more suitable ss NA building block linkers, wherein e.g., a ss oligo linker is used, to allow assembling a pair of NA building blocks (e.g., both ss or ds, or one being ss and the other one being ds) that would not hybridize to each other without using the linker(s).

For direct assembly, NA building blocks are joined together by their single stranded parts or overlaps (i.e., the overlapping parts or overhangs), such that the overlaps are included in the continuous sequence only once. Upon aligning two NA building blocks with an overlap, a continuous sequence is formed which has a length that is the length of both individual NA building blocks taken together, minus the length of the overlap. Consequently, a continuous sequence is obtained which comprises a segment of each of the aligned NA building blocks.

For indirect assembly, the target ds polynucleotide or any intermediates can be formed upon assembly of ss NA building blocks and joining them through one or more ss linker(s). For example, two ss NA building blocks, each of e.g., 10 bases length, may be joined by a ss oligo linker of e.g., 6 bases length, such that 3 bases of the 3′ terminal end of the first ss NA building block align with the 3 bases of the 5′ end of the ss linker and that 3 bases of the 5′ end of the second ss NA building block align with the 3 bases of the 3′ end of the ss linker.

Single stranded oligo linkers having a sequence complimentary to the combined overhangs may connect adjacent NA building blocks in the target polynucleotide. Linkers may e.g., consist of 6 bases to connect two adjacent NA building blocks, each with a 3 base long overhang, one on the 3′ end and the other on the 5′ end, respectively.

Each assembly will result in an extension of one or both ends of an NA building block, such as an extension of the 5′-end and/or the 3′-end of an NA building bock.

Multiple parallel reactions can be carried out. Specifically, an assembly tier is characterized by at least two assembly reactions in parallel, in particular wherein the respective reaction products will be further reacted in one or more further steps. Specifically, one or more parallel assembly reactions can be performed in separation reaction containments during one assembly period (“tier” or “round”), before one or more further assembly reactions are performed in any of the following tiers, using assembly product(s) of any of the previous tiers.

The term “single-tier” refers to a single compartment assembly method, such as to allow pooled assembly of NA building blocks. For example, all starting oligos can be assembled in a pooled way i.e., at the same time in one reaction containment (“one pot”).

The term “multiple-tier” refers to a sequential multi compartment assembly method, where assembly reactions are performed in parallel during one tier, and reaction products obtained by such parallel assembly reactions are used as intermediates to perform further assembly steps in one or more further tiers of assembly.

Specifically, the method described herein is a multiple-tier assembly method to produce the ds target polynucleotide in more than one tiers of assembly reactions e.g., at least two, three, four, five, six, seven, eight, nine, or ten tiers, or even more than 10 tiers such as at least 11, 12, 13, 14, 15, 16, 17, 18, 19, or 20, up to a number of tiers which is conveniently used in the synthesis of long target polynucleotides e.g., up to 30, or up to 20, or up to 10 tiers.

Assembly tiers are typically sequential. Specifically, an assembly tier starting with assembly of only starting NA building blocks is termed “tier 1”. Assembly of reaction products obtained by such tier 1 assembly, are typically used in one or more further tiers of assembly, which can be consecutive or non-consecutive.

Multiple-tier assembly is herein specifically understood as a multi-part, modular assembly method. Contrary to single-tier or “one-pot” type of assembly, it particularly refers to a hierarchical or sequential assembly, where the component DNA pieces are assembled in.

Multi-tier DNA assembly methods can be performed with methods that combine or include e.g., Golden Gate assembly, BASIC, BioBrick assembly (and variants such as BglBrick) and Gateway cloning. Bespoke multi-tier DNA assembly methods can also include Gibson Assembly, AQUA cloning, Twin Primer Assembly, ligase cycling reaction, SLIC, SLICE, overlap extension PCR and CPEC.

To compare, single-tier assembly is understood to assembly a polynucleotide in one step, e.g., using pool reactions or “one-pot” assembly reactions.

The term “assembly workflow” or simply “workflow” as used herein refers to the pre-defined sequence of assembly of NA building blocks to the target ds polynucleotide. The workflow is typically optimized to allow efficient synthesis of the target polynucleotide, aiming to avoid mismatches of NA building blocks which would lead to a polynucleotide comprising an incorrect sequence.

The workflow is specifically designed to avoid mismatches or reaction products which cannot be used for assembly to produce the target ds polynucleotide. If there are partial constructs that can anneal in alternative ways, a runaway i.e., an uncontrolled polymerization reaction, can occur. To avoid combinations of pairs of matching NA building blocks that would result in unwanted constructs or runaway reactions, the pairs of matching NA building blocks are assembled in a predetermined sequence of assembly steps i.e., a specific workflow. Preferably, said specific workflow is not linear but hierarchical i.e., following an algorithm that provides for intermediate reaction products which are defined non-consecutive parts of the target ds polynucleotide conveniently produced avoiding undesired reaction products to the extent possible, before such intermediate reaction products are further assembled into further intermediate reaction products or into the target ds polynucleotide sequence.

In a linear workflow, the polynucleotide is assembled in a linear fashion starting at the 3′ end of the leading strand, and adding the next oligo to link the 3′ end of the leading strand with the 5′ end of the next oligo. For example, oligo B is ligated to oligo A, oligo C is ligated to oligo B, oligo D is ligated to oligo C and so forth. This assembly may be achieved simultaneously by adding all oligos to the reaction containment at the same time (such as in a pool assembly), or the polynucleotide is extended progressively by successively adding oligos A, B, C, D and so forth to the reaction containment.

Specifically, a linear assembly workflow employs only a starting oligo at a time, reacting it with an intermediate product (a reaction product of the previous tier) of the growing ds polynucleotide. In this case there are as many tiers as starting oligos, always using a starting oligo at each “tier”.

In contrast, a hierarchical assembly of NA building blocks, is a tiered molecular assembly where at least two reaction products of a previous tier are used in further tiers.

In a hierarchical workflow, the polynucleotide is typically assembled in at least two tiers. To generate a target polynucleotide A-B-C-D, in the first tier, oligo B is ligated to oligo A, oligo D is ligated to oligo C, producing the reaction products A-B and C-D. In the second tier, the reaction product A-B is ligated to C-D, thereby producing the target polynucleotide A-B-C-D.

However, undesired reaction products may be obtained where oligos A and B are capable of hybridizing not only in the orientation A-B but also B-A due to complimentary sequences or overhangs. A linear hierarchical workflow as described above would result in the unwanted polynucleotide A-B-A-B-C-D, and fragments like A-B-A or B-A, in addition to the desired polynucleotide A-B-C-D. Such unwanted side reactions drastically reduce the yield and purity of the target polynucleotide.

According to a specific aspect described herein, an asymmetric hierarchical workflow is employed. Accordingly, in the above example, only oligos B and C are ligated in a first tier, producing the ds NA building block B-C. In a second tier, the ds NA building block B-C is reacted with the ss NA building block D to produce the intermediate polynucleotide B-C-D. The intermediate polynucleotide B-C-D is then connected with the ss building block A, wherein upon ligation the desired polynucleotide A-B-C-D is formed.

Geometric (also called “symmetric”) assembly is a type of hierarchical assembly where at every step, pairs of assembly products of a same previous tier are used, thereby halving (or nearly halving, in case of odd number of reactions) the number of NA building blocks to be assembled, at every tier, see specific Examples A and B below.

Example A: 16 reaction products of previous tier are used, to obtain 8 intermediates, and then these 8 intermediates are used to get further 4 intermediates, in turn used to get 2 intermediates and a final reaction of 1 molecule (the target one). In this case, 8 starting oligos were used in the first tier, to result in 4 intermediates which are assembled in the second tier, to result in 2 intermediates which are assembled in the third tier, to result in one target polynucleotide.

Example B: Two reaction products of a tier 1 and two reaction products of tier 2 are assembled in parallel, to get two intermediates (of different length). In a following tier, the two intermediates are assembled to obtain a target molecule. In each step, the assembly was symmetric, because the number of NA building blocks used in each previous tier was reduced by half.

According to a specific aspect as described herein, a specific type of hierarchical assembly is performed which is an asymmetric (also called “non-geometric”) assembly. In such asymmetric assembly method, NA building blocks are assembled in multiple assembly tiers, wherein in at least one tier, an asymmetric pair of NA building blocks is assembled; see specific Example C below

Example C: in a first reaction, a reaction product of tier 3 is assembled with a starting oligo, and in parallel, in a second reaction, a reaction product of tier 2 is assembled with a reaction product of tier 3, followed by another tier where in a third reaction, the reaction products of the first and second reactions are combined (assembled), and in yet another tier, the reaction product of the third reaction is reacted with another NA building block (from any tier).

Specifically, at least one NA building block of the asymmetric pair of NA building blocks is a starting oligo (e.g., a ss oligo), or a reaction product from an earlier tier where at any assembly tier reaction product of any previous tier (not the immediate previous tier), is used.

According to as specific aspect, as described herein, an asymmetric, hierarchical assembly workflow is employed, which comprises the assembly of at least one asymmetric pair of NA building blocks. Such asymmetric pair of NA building blocks particularly consists of two NS building blocks of different length, which originate from different assembly tiers.

The asymmetric, hierarchical assembly method can involve also reacting two products of the same previous tier, but not every reaction at every tier is composed from reaction products of the previous tier. In this case, the number of reaction products is not halved at each tier.

Specifically, NA building blocks can be assembled in one particular tier, wherein at least one NA building block originates from an assembly tier other than the preceding tier. For example, at least one NA building block is assembled with another NA building block, wherein said NA building blocks originate from tiers that are at least one or more e.g., at least two, three, four, five, six, seven, eight, nine, or ten tiers different.

According to a specific example of a multi-tier assembly method, one NA building block of the asymmetric pair of NA building blocks is a starting oligo e.g., an ss oligo, and the other NA building block of the asymmetric pair of NA building blocks, can be a reaction product (e.g., a ds NA building block) of any preceding tier.

According to a specific example, one of the NA building blocks is a starting NA building block, and the other NA building block that is assembled with the starting NA building block, has been obtained as a reaction product of the tier 1 assembly, or of any of the further tiers of assembly, up to the immediate preceding assembly tier which results in the ds target polynucleotide.

According to another specific example of a multi-tier assembly method, the two NA building blocks can be ds NA building blocks which are each reaction products from assembly reactions of different tiers

Specifically, unwanted side products of assembly can be avoided by changing the length of the respective NA building blocks thereby changing the overhang sequence. However, changing the length or the sequence of oligos is often not possible or desired, e.g., when oligos are derived from restriction digest or from a library comprising oligos of a specific length only, making assembly of the desired polynucleotide difficult or impossible.

In one specific embodiment described herein, the process of determining the assembly workflow is carried out by an algorithm. Candidate divisions of the sequence of the template are systematically examined to find the optimal number, length of the NA building blocks of pairs of NA building blocks (including e.g., asymmetric pairs of different length), and assembly sequence of subsets to divide it into for synthesis in accordance with the method provided herein. Initially, the entire target sequence can be taken as a single subset, after which smaller and smaller subsets are formed with increasing numbers of candidate NA building blocks in decreasing size until a partitioning is found that fulfills the subset criteria listed above.

In the method provided herein, a SOI which is the sequence of a target polynucleotide or the sequence of a template thereof, may be divided into sub-sequences, corresponding to subsets of NA building blocks, avoiding particular nucleotide synthesis problems, such as palindromic sequences, runaway reactions and unambiguous assembly. In particular, such division into shorter NA building blocks may be very efficient to shorten the assembly process and to avoid the need of separating unwanted reaction products. Specifically, ligation of subsets of NA building blocks yields intermediate reaction products, also called intermediates, and assembly of intermediate reaction products ultimately yields the target ds polynucleotide. Preferably, additional criteria to those listed above may be used for selecting subsets of NA building blocks. Such additional criteria include, but are not limited to, minimization of the size of the subset of NA building blocks employed in any single ligation reaction (for example to avoid mismatch ligations), minimizing the difference in annealing temperature of members of a subset of NA building block precursors, minimizing the difference in annealing temperatures of the overhangs of different ds subunits, whether to employ frame-shifting adaptors or ss oligo linkers and whether to minimize the degree of cross-hybridization among the hybrid forming portions of different NA building blocks that make up a subset.

Specifically, the method described herein employs an “asymmetric assembly workflow”, comprising multiple assembly tiers, wherein in at least one assembly tier at least one asymmetric pair of NA building blocks is assembled. To avoid combinations of pairs of matching NA building blocks that would result in unwanted constructs or runaway reactions, pairs of matching NA building blocks are assembled in a predetermined sequence of asymmetric assembly steps. Preferably, said specific workflow follows an algorithm that provides for intermediate reaction products which are defined non-consecutive parts of the target ds polynucleotide conveniently produced avoiding undesired reaction products to the extent possible, before such intermediate reaction products are further assembled into further intermediate reaction products or into the target ds polynucleotide sequence.

The term “asymmetric pair of NA building blocks” as used herein shall refer to two NA building blocks, which differ in length. Specifically, said asymmetric pair of NA building blocks is assembled in one assembly tier, optionally followed by assembly of a further asymmetric pair of NA building blocks in a further assembly tier, wherein one of said NA building blocks of said further asymmetric pair is a product of assembly of said asymmetric pair in the previous assembly tier. In an asymmetric workflow, at least one pair of asymmetric NA building blocks is assembled in at least one tier, preferably in at least two or more tiers asymmetric pairs of NA building blocks are assembled.

In a symmetric workflow, NA building blocks of equal length are assembled e.g., in each assembly tier.

A nucleic acid (NA) building block is herein particularly understood as an oligonucleotide or polynucleotide which is used in the assembly of the target polynucleotide described herein. NA building blocks may be single stranded or double stranded oligonucleotides or polynucleotides as described herein. Upon assembly in multiple tiers, they form the target polynucleotide.

“Pairs of matching NA building blocks” are herein understood as two (in some particular cases more than two, e.g., three) NA building blocks which comprise matching nucleotide sequences that are complimentary to each other, or at least partially complementary to each other, such as to allow ligation or a hybridization reaction between the matching nucleotide sequences. Sufficiently complementary nucleotide sequences can be at least 50%, 60%, 70%, 80%, more 90%, 95%, 96%, 97%, 98%, 99%, or 100% complementary to each other.

Specifically, matching nucleotide sequences are of regions of two single stranded nucleic acids or overhang parts of ds nucleic acids, and have a nucleotide base composition that allow the respective single-stranded regions to anneal to a stable, double-stranded hydrogen-bonded region, in particular applying stringent annealing conditions, such annealing is also referred to as “hybridization”. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotide sequences are 100% complementary. Besides conventional bases (A, G, C, T), analogs e.g., inosine and 2′-deoxyinosine and their derivatives (e.g. 7′-deaza-2′-deoxyinosine, 2′-deaza-2′-deoxyinosine), azole- (e.g. benzimidazole, indole, 5-fluoroindole) or nitroazole analogues (e.g. 3-nitropyrrol, 5-nitroindol, 5-nitroimidazole, 4-nitropyrazole, 4-nitrobenzimidazole) and their derivatives, acyclic sugar analogues (e.g. those derived from hypoxanthine- or indazole derivatives, 3-nitroimidazole, or imidazole-4,5-dicarboxamide), 5′-triphosphates of universal base analogues (e.g. derived from indole derivatives), isocarbostyril and other hydrophobic analogues, and any of its derivatives (e.g. methylisocarbostyril, 7-propynylisocarbostyril), hydrogen bonding universal base analogues (e.g. pyrrolopyrimidin), and other chemically modified bases (such as diaminopurine, 5-methylcytosine, isoguanine, 5-methyl-isocytosine, K-2′-deoxyribose, P-2′-deoxyribose) can have different base-pairing preferences and can pair with more than one natural nucleobase with similar stringency/probability. In certain cases, the monomers are linked by phosphodiester or by peptidyl linkages or by phosphorothioate linkages.

The term “single stranded (ss) NA building block”, shall refer to a ss oligonucleotide or polynucleotide of a DNA (herein also referred to as “ssDNA”), which is a linear polymer of nucleotide monomers. Monomers making up oligonucleotides or polynucleotide are capable of specifically binding to a natural polynucleotide by way of a regular pattern of monomer-to-monomer interactions, such as Watson-Crick type of base pairing, base stacking, Hoogsteen or reverse Hoogsteen types of base pairing, wobble base pairing, or the like.

Ss NA building blocks described herein, which are oligonucleotides, typically range in size between 6 and 26, but may be longer e.g., between 6 and 220 nucleotides, such as at least 6, 7, 8, 9, 10, 11, 12, 13, 14, 15, 16, 17, 18, 19, 20, 21, 22, 23, 24, 25, 26, or 27, up to 220, 210, 200, 190, 180, 170, 160, 150, 140, 130, 120, 110, 100, 90, 80, 70, 60, 50, 40, 30, 29, 28, 27, or 26 nucleotides. Whenever an oligonucleotide is represented by a sequence of letters (upper or lower case), such as “ATGC,” it will be understood that the nucleotides are in 5′→3′ order from left to right and that “A” denotes deoxyadenosine, “T” denotes deoxythymidine, “G” denotes deoxyguanosine, and “C” denotes deoxycytidine. Besides conventional nucleotides (A, G, C, T), modified nucleotides e.g. K-2′-deoxyribose, P-2′-deoxyribose, 2′-deoxyinosine, 2′-deoxyxanthosine or nucleotides with nucleobase analogs may be used e.g., inosine, or 5-methylisocytosine, or 3-nitropyrrole, 5-nitroindole, pyrrolidine, 4-nitroimidazole, 4-nitropyrazole, 4-nitrobenzimidazole, 4-aminobenzimidazole, 5-nitroindazole, 3-nitroimidazole, 5-aminoindole, benzimidazole, 5-fluoroindole, indole, methylisocarbostyril, pyrrolopyrimidine 7-propynylisocarbostryril. The terminology and atom numbering conventions follow those disclosed in Strachan and Read, Human Molecular Genetics 2 (Wiley-Liss, New York, 1999). Usually, an oligonucleotide or polynucleotide comprises the four natural nucleosides (e.g., deoxyadenosine, deoxycytidine, deoxyguanosine, deoxythymidine for DNA or their ribose counterparts for RNA) linked by phosphodiester or by peptidyl linkages or by phosphorothioate linkages; however, they may also comprise non-natural nucleotide analogs e.g., including modified bases, sugars, or internucleosidic linkages.

Ss NA building blocks, such as oligonucleotides, can be produced using chemical synthesis methods e.g., by synthesizing the oligonucleotide sequence from monomer-phosphoramidites, dimer-phosphoramidites (Neuner, Cortese, and Monaci 1998) or trimer-phosphoramidites (Sondek and Shortle 1992), mixture of monomer-phosphoramidites, mixture of dimer-phosphoramidites, mixture of trimer-phosphoramidites or their combination thereof.

In some embodiments, NA building blocks are produced and purified from naturally-occurring sources, or synthesized in vivo, within the cell undergoing in vivo mutagenesis using any of a variety of well-known enzymatic methods e.g., as described in Farzadfard et al. (2014). Specifically, enzymes that synthesize soft-randomized NA building blocks include, but are not limited to low fidelity DNA polymerase proteins or low fidelity reverse transcriptase proteins which incorporate mismatching nucleotides during synthesis with high frequency. Alternatively, mismatching nucleotides are incorporated into the NA building blocks with a higher frequency by the DNA polymerases or reverse transcriptases due to the presence of chemical substances, which are well-known to those skilled in the art.

The term “double stranded (ds) NA building block” as used herein, shall refer to a NA building block which is a linear polymer of nucleotide dimers. Dimers making up oligonucleotides or polynucleotides comprise complementary nucleotides bound by way of a regular pattern of monomer-to-monomer interactions, such as Watson-Crick type of base pairing, base stacking, Hoogsteen or reverse Hoogsteen types of base pairing, wobble base pairing, or the like.

Double stranded NA building blocks described herein, which are oligonucleotides, typically range in size between 6 and 26 base pairs (bp), but may be longer. dsDNA oligonucleotides described herein may range in size between 6 and 200 base pairs, e.g., between 6 and 220 base pairs, such as at least 6, 7, 8, 9, 10, 11, 12, 13, 14, 15, 16, 17, 18, 19, 20, 21, 22, 23, 24, 25, 26, or 27, up to 220, 210, 200, 190, 180, 170, 160, 150, 140, 130, 120, 110, 100, 90, 80, 70, 60, 50, 40, 30, 29, 28, 27, or 26 base pairs. Whenever an oligonucleotide or polynucleotide is represented by a sequence of letters (upper or lower case), such as “ATGC,” it will be understood that the nucleotides are in 5′→3′ order from left to right and that “A” denotes deoxyadenosine, “T” denotes deoxythymidine, “G” denotes deoxyguanosine, and “C” denotes deoxycytidine. Besides conventional nucleotides (A, G, C, T), modified nucleotides e.g. K-2′-deoxyribose, P-2′-deoxyribose, 2′-deoxyinosine, 2′-deoxyxanthosine or nucleotides with nucleobase analogs may be used e.g., inosine, or 5-methylisocytosine, or 3-nitropyrrole, 5-nitroindole, pyrrolidine, 4-nitroimidazole, 4-nitropyrazole, 4-nitrobenzimidazole, 4-aminobenzimidazole, 5-nitroindazole, 3-nitroimidazole, 5-aminoindole, benzimidazole, 5-fluoroindole, indole, methylisocarbostyril, pyrrolopyrimidine 7-propynylisocarbostryril. The terminology and atom numbering conventions follow those disclosed in Strachan and Read, Human Molecular Genetics 2 (Wiley-Liss, New York, 1999). Usually, an oligonucleotide or polynucleotide comprises the four natural nucleosides (e.g. deoxyadenosine, deoxycytidine, deoxyguanosine, deoxythymidine for DNA or their ribose counterparts for RNA) linked by phosphodiester or by peptidyl linkages or by phosphorothioate linkages; however, they may also comprise non-natural nucleotide analogs, e.g. including modified bases, sugars, or internucleosidic linkages.

The simplest end of a double stranded molecule is called a blunt end. In a blunt-ended molecule, both strands terminate in a base pair. Non-blunt ends are created by various overhangs. The term “overhang” as used herein refers to a stretch of unpaired nucleotides at an end of a ds NA building block molecule. These unpaired nucleotides can be in either strand, creating either 3′ or 5′ overhangs.

An overhang is specifically characterized by a ss terminal stretch of one or more nucleotides that is reactive to hybridize with a matching ss sequence of a reaction partner (such as another ss or ds NA building block), which ss terminal stretch is extending the double stranded part of the ds NA building block. In particular, the overhang is reactive insofar that it is capable of hybridizing with another ss oligo or overhang that comprises a matching sequence. A reactive overhang is herein also understood to result in a “sticky end” of the molecule.

The overhang can be part of the leading strand or the lagging strand.

Where the overhang is at the 5′-end of the ds NA building block and is part of the leading strand, the overhang comprises the 5′-terminal region including the 5′-terminus of the leading strand.

Where the overhang is at the 3′-end of the ds NA building block and is part of the leading strand, the overhang comprises the 3′-terminal region including the 3′-terminus of the leading strand.

Where the overhang is at the 5′-end of the ds NA building block and is part of the lagging strand, the overhang comprises the 3′-terminal region including the 3′-terminus of the lagging strand.

Where the overhang is at the 3′-end of the ds NA building block and is part of the lagging strand, the overhang comprises the 5′-terminal region including the 5′-terminus of the lagging strand.

Specifically, a ds NA building block may be used which comprises one overhang at one end, and a blunt end at the other end. A blunt end is specifically characterized by a ds terminal stretch of one or more base pairs which includes the terminus of both strands without an overhang at such end of the ds NA building block.

An overhang may consist of one nucleotide, or can be longer. An overhang may comprise or consist of any one of 1, 2, 3, 4, 5, 6, 7, 8, 9, 10, 11 or 12 nucleotides, or at least any one of 1, 2, 3, 4, 5, 6, 7, 8, 9, 10, 11 or 12 nucleotides. An overhang is typically not more than half of a ds NA building block length. For example, if said ds NA building block is a ds oligo that is 6 nucleotides long (in the ds part of the oligo), the overhang is typically not more than 3 nucleotides long, meaning the overhang can also be 1 or 2 nucleotides long. According to a specific example, if said ds oligo is 24 nucleotides long (in the ds part of the oligo), the overhang is typically not more than 12 nucleotides long, meaning it can also be 1, 2, 3, 4, 5, 6, 7, 8, 9, 10 or 11 nucleotides long.

The term “derivative” refers to an oligonucleotide or a polynucleotide differing from the original oligonucleotide or polynucleotide, but retaining essential properties thereof. Derivatives may e.g., be produced using a ds polynucleotide (e.g. DNA) as a starting material to engineer single stranded DNA, or complementary RNA molecule, to introduce one or more point mutations, or to bind heterologous moieties or tags by chemical and/or enzymatic means.

Generally, derivatives are overall closely similar, and, in many regions, identical to the original oligonucleotide or polynucleotide. As a practical matter, whether any particular nucleic acid molecule or polypeptide is at least 90%, 91%, 92%, 93%, 94%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

The terms “hybridize,” “hybridization,” “hybridizing,” “anneal,” and “annealing,” as used herein, generally refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of a PCR, or the enzymatic cleavage of a polynucleotide by a ribozyme.

The term “intermediate polynucleotide” as used herein, refers to a product of the assembly of two NA building blocks. For example, a pair of matching NA building blocks is transferred into a reaction containment and the matching NA building blocks are assembled thereby forming a new NA building block that is double stranded and considered as an intermediate because used in a further assembly step. Specifically, said ds NA building block may comprise at least one overhang. Such overhang allows further assembly with another matching NA building block in the direction of the overhang. Where an intermediate comprises two overhangs i.e., on both of its ends, such intermediate is designed to assemble with further NA building blocks in both directions.

As used herein, the term “ligation” is intended to mean the process during which two nucleic acid sequences anneal to one another and/or assemble with intermolecular chemical bonds (e.g., hydrogen bonds) so as to form a double strand under appropriate conditions.

Ligation products, herein also referred to as reaction products, can be formed from both double stranded nucleic acids and single stranded nucleic acids. Double-stranded nucleic acids can be ligated by “sticky end” ligation or “blunt end” ligation. In sticky end ligation, staggered ends comprising terminal overhangs can hybridize to a ligation partner. In blunt end ligation, terminal overhangs are not present and successful ligation depends on transient associations of 5′-ends and 3′-ends. Blunt end ligations in general are less efficient than sticky end ligations, and various optimizations, such as adjusting concentrations, incubation times, and temperatures, can be applied to improve efficiencies. Single-stranded polynucleotides can also be ligated.

The ligation efficiency between two complementary sequences or sufficiently complementary sequences depends on the operating conditions that are used, and in particular the stringency. The stringency may be understood to denote the degree of homology; the higher the stringency, the higher percent homology between the sequences. The stringency may be defined in particular by the base composition of the two nucleic sequences, and/or by the degree of mismatching between these two nucleic sequences. By varying the conditions e.g., salt concentration and temperature, a given nucleic acid sequence may be allowed to ligate only with its exact complement (high stringency) or with any somewhat related sequences (low stringency). Increasing the temperature or decreasing the salt concentration may tend to increase the selectivity of a ligation reaction.

The ligation reaction can be performed by hybridizing nucleic acid molecules which are sufficiently complementary to each other.

The ligation reaction can be performed by an enzyme, specifically a DNA ligase enzyme. A DNA ligase catalyzes the formation of covalent phosphodiester linkages, which permanently join the nucleotides together. In addition, T4 DNA ligase can also ligate ssDNA if no dsDNA templates are present, although this is generally a slow reaction. Non-limiting examples of enzymes that can be used for ligation reactions are ATP-dependent double-stranded polynucleotide ligases, NAD+ dependent DNA or RNA ligases, and single-strand polynucleotide ligases. Non-limiting examples of ligases are Escherichia coli DNA ligase, Thermus filiformis DNA ligase, Thermus thermophilus DNA ligase, Thermus scotoductus DNA ligase (I and II), CircLigase™ (Epicentre; Madison, WI), T3 DNA ligase, T4 DNA ligase, T4 RNA ligase, T7 DNA ligase, Taq ligase, Ampligase (Epicentre® Technologies Corp.), VanC-type ligase, 9° N DNA Ligase, Tsp DNA ligase, DNA ligase I, DNA ligase III, DNA ligase IV, Sso7-T3 DNA ligase, Sso7-T4 DNA ligase, Sso7-T7 DNA ligase, Sso7-Taq DNA ligase, Sso7-E. coli DNA ligase, Sso7-Amp ligase DNA ligase, and thermostable ligases. Ligase enzymes may be wild-type, mutant isoforms, and genetically engineered variants. Ligation reactions can contain a buffer component, small molecule ligation enhancers, and other reaction components.

Specifically, a T4 DNA ligase can be used in a ligation reaction. In the method provided herein, the ligation reaction can be performed under high-fidelity conditions that block side reactions and minimize mismatches.

Assembly of NA building blocks into intermediate polynucleotides or into the target polynucleotide may be carried out using suitable ligation buffer solutions. The ligation buffer solution is e.g., an aqueous solution, typically in a nuclease-free environment, at a pH that ensures the selected ligase will be active; typically, this is a pH of between about 7-9. Preferably, the pH is maintained by Tris-HCl at a concentration of between about 5 mM to 50 mM. The ligation buffer solution may include one or more nuclease inhibitors, usually calcium ion chelators, such as EDTA. Typically, EDTA is included at a concentration of between about 0.1 to 10 mM. The ligation buffer solution includes whatever cofactors are required for the selected ligase to be active. Usually, this is a divalent magnesium ion at a concentration of between about 0.2 mM to 20 mM, typically provided as a chloride salt. For T4 DNA ligase ATP is required as a cofactor. The ligase buffer solution may also include a reducing agent, such as dithiothreitol (DTT) or dithioerythritol (DTE), typically at a concentration of between about 0.1 mM to about 10 mM. Optionally, the ligase buffer may contain agents to reduce nonspecific binding of the oligonucleotides and polynucleotides. Exemplary agents include salmon sperm DNA, herring sperm DNA, serum albumin, Denhardt's solution, and the like. Preferably, ligation conditions are adjusted so that ligation will occur if matching NA building blocks form perfectly matched duplexes with the bases of a contiguous complementary region. However, it is understood that it may be advantageous to permit non-pairing nucleotides on the 5′-end of one NA building block of a matching pair of NA building blocks and the 3′-end of the other one of the matching pair of NA building blocks to aid in detection or to reduce blunt-end ligation. Important parameters in the ligation reaction include temperature, salt concentration, presence or absence and concentration of denaturants such as formamide, concentration of the pair of NA building blocks and type of ligase employed. Methods of selecting hybridization conditions for the reaction are known to those skilled in the art.

Preferably, ligation is performed under stringent hybridization conditions to ensure that only matching NA building blocks hybridize. Typically, stringency is controlled by adjusting the temperature at which hybridization occurs while holding salt concentration at some constant value e.g., at about (+/−20%) 100 mM NaCl, or the equivalent salt concentration of other salts. Other factors can be relevant, such as the particular sequence or length of the matching pair of NA building blocks, and/or the heat lability of the selected ligase. Preferably, the ligation reaction is carried out at a temperature close to the melting temperature of hybridized NA building blocks in the ligation buffer. More preferably, the ligation reaction is carried out at a temperature within 10° C. below the melting temperature of hybridized NA building blocks in the ligation buffer solution. Most preferably, the ligation reaction is carried out at a temperature in the range of 0 to 5° C. below the melting temperature of hybridized NA building blocks in the ligation buffer solution.

Ligation may be followed by one or more amplification reactions. In some embodiments, the ligation products, or target polynucleotides are isolated or enriched prior to amplification.

Isolation can be achieved by various suitable purification methods including affinity purification and gel electrophoresis. For example, ligation products, or target polynucleotides can be isolated by binding of a selective binding agent immobilized on a support to a tag attached to the capture probe. The support can then be used to separate or isolate the capture probe and any polynucleotide hybridized to the capture probe from the other contents of the sample reaction volume. The isolated polynucleotides can then be used for amplification and further sample preparation steps. In some embodiments, the capture probe is degraded or selectively removed prior to amplification of the circular target polynucleotides. Amplification of reaction products, or target polynucleotides can be achieved by various suitable amplification methods known to those skilled in the art.

Reaction and storage compartments may be conveniently provided within one or more parts of a device, which together are provided as “array device”. Such array device may be any one or more of a microtiter plate, microfluidic microplate, set of capillaries, microarray or a biochip, preferably a DNA or RNA biochip.

A microarray is herein understood as a supporting material (such as a glass or plastic slide) onto which numerous molecules or fragments such as DNA or NA building blocks are attached in a regular pattern. More specifically, it refers to microscope slides that are printed with thousands of tiny spots in defined positions, wherein said spots are capable of binding DNA. Such slides are often also referred to as biochips, DNA chips, or gene chips. Microarrays can bind DNA in a covalent or non-covalent manner and can thus serve as array devices in which NA building blocks are stored in pre-defined locations, i.e., spots.

In a specific example, said separate library containments are micro-well plates, arranged as stacked plates, optionally barcode labelled, and accessible by an automated microdroplet handler. Library members may be conveniently stored in said stacked micro-well plates, wherein the order and stacking is according to decreasing frequency of use. One example is to store into micro-well plates where the first plate comprises the most common pair combinations of NA building blocks, in decreasing order until the last micro-well plate which contains the least-frequently used library members.

According to a specific aspect, a microfluidics chip can be used in order to pipe droplets containing NA building blocks in such a way as to implement the assembly workflow. The design of the chip can be that of a symmetric workflow, but by leaving certain channels free of the NA building blocks, an asymmetric workflow is enabled. Similarly, digital microfluidics e.g., platforms of electro-wetting on dielectric (EWOD) can also be used to have accurate control on the movement of individual droplets, thereby facilitating the implementation of complex hierarchical workflows.

According to a specific aspect, a microtiter plate can be used, which term is understood to refer to well plates, multi-well plates or micro-well plates. These plates are commonly manufactured in a 2:3 rectangular mix with 96, 384, or 1536 wells, although other cavity configurations are available. Some of the other sizes, far less common, available are 6, 24, 3456, and 9600 wells. The wells of the microplate typically hold between tens of nanoliters to several milliliters of liquid.

Capillaries can be any glass capillaries, microfluidic capillaries and autonomous microfluidic capillary systems. Capillary microfluidics are important tools in many different fields. Due to their axisymmetric flow and ability to withstand organic solvents, when compared with their lithographically fabricated polydimethylsiloxane (PDMS) counterparts, glass capillary devices possess advantages for microfluidic applications. In particular, a circular tube is inserted into a square outer flow channel, which greatly simplifies alignment and centering of these devices. These devices can produce small and large droplets, ranging from 10 to multiple hundreds in μm size.

NA building blocks may be conveniently transferred from an array device to reaction containments by automated means e.g., either robotically or via dedicated fluids using, for example, an automated liquid handler, from such compartments into other compartments herein referred to as reaction compartments, i.e. from one vessel to another. In order to facilitate time efficient assembly of polynucleotides, hierarchies of reactions and respective vessels may be employed corresponding to frequency of use of NA building block library members. The transfer to a new vessel involves the physical movement of a device that picks one or more molecules of a NA building block from the respective location, or the pneumatic/hydraulic deposition though microfluidics. Due to the large number of NA building blocks required to theoretically build any given sequence, most spatial distributions would incur into wasted time and resources due to scanning and lengthy travel times of the liquid handler. However, by using a specific distribution of the library members or reaction compartments, it can be ensured that that there is minimal movement according to a target sequence.

According to a specific aspect, transfer of NA building blocks described herein, such as to transfer from one containment to another, e.g., transfer from a library containment to a reaction containment, is done using an automated system e.g., using a liquid handler such as further described herein.

As used herein the terms “liquid handler”, “automated handler” or “microdroplet handler” refer to any device used in a method of liquid handling, preferably, automated liquid handling, preferably a device as used in sensor-integrated robotic systems. As low-volume dispensing becomes increasingly common in life science, microsyringes have emerged which have a high level of precision with hermetic seals. Some manual or electronic holders are designed to precisely control the piston displacement to ensure the accuracy of the dispensed volume. Besides the syringe, a pipette is another popular tool for liquid handling. The dispensed volume can be at the micro- or sub-microliter level. Multichannel pipettes are recommended for multirouting pipetting at one time. There are both fixed- and adjustable-volume pipettes on the market. The former is more accurate and precise, whereas the latter has a larger scope of applications because the operator can choose different volumes according to need. Besides, high throughput has become important in life science research. One of the representative applications is microarray printing. This technology creates an array of biosample spots each at the nanoliter scale to enable the analysis of large numbers of experiments in parallel with only tiny quantities of samples. The process of spotting thousands of biosamples is almost an impossible task with a handheld dispensing tool, making robotic liquid handling an important aspect.

Robotic workstations have multiple advantages over manual liquid handling since robots can work without fatigue, increase the throughput, perform consistently, and ensure accuracy and precision. According to the requirements for the platform with integration and multifunction, there are still more complex systems in which the liquid-handling task is only one part of the function. The generic architecture of liquid-handling may be built up as follows. First, the control center controls a robot that moves between the dispensing part and the washing station of the robotic workstation. The washing station is used to clean the dispensing head for lengthening its life and for ensuring the safety of the sample. Liquid samples are expelled from the dispensing head and deposited on the substrates for further processing. Sensors are incorporated to monitor the status of the dispensing part such that feedback control can be performed by the control center. Sensors are not always installed on all the workstations but are more and more used to construct the feedback loop for delivering a better performance.

Specifically, a liquid handler can be used in a method described herein, in particular a microdroplet handler. Specifically, the liquid handler is automated. Using a liquid handler, a suitable volume of at least any one of 10, 20, 30, 40, 50, 60, 70, 80, 90 100, 200 or 500 nL can be transferred. Typically, at least any one of 10⁹, 10¹⁰, 10¹¹ or 10¹² copies of a library member e.g., NA building blocks sourced from one library containment, or matching pairs of NA building blocks sourced from separate library containments, are placed into one reaction containment. For example, at least about 10¹¹ copies (e.g., 6.06×10¹¹ copies) of specific NA building blocks are placed into one reaction containment to react with each other. Typically, the volume in which NA building blocks are transferred by a liquid handler is between 10 and 1000 nL. Preferably it is between 10 and 500 nL, and more preferably it is between 50 and 250 nL.

“Microfluidic devices” typically enable the manipulation of discrete fluid packets in the form of microdroplets that provide numerous benefits for conducting biological and chemical assays. Among these benefits are a large reduction in the volume of reagent required for assays, the size of sample required, and the size of the equipment itself. Such technology also enhances the speed of biological and chemical assays by reducing the volumes over which processes such as heating, diffusion, and convective mixing occur. Once the droplets are generated, carefully designed droplet operations allow for the multiplexing of a large number of droplets to enable large-scale complex biological and chemical assays.

The term “microfluidic microplate” as used herein, refers to a combination of microfluidic technology with standard SBS-configured 96-well microplate architecture, in the form of microfluidic microplate technology. A microfluidic microplate allows for the improvement of essential workflows, conservation of samples and reagents, improved reaction kinetics, and the ability to improve the sensitivity of the assay by multiple analyte loading.

The term “library” as used herein shall refer to a collection of library members which are NA building blocks (e.g., an oligonucleotide or polynucleotide library). Library members can be ss NA building blocks or ds NA building blocks. A library typically contains library members which are diverse. The library described herein comprises library members suitably composed of NA building blocks of varying lengths and different sequences.

According to a specific example, the library is a focused library which is designed to assemble a pre-defined target polynucleotide e.g., according to a template or comprising a SOI, wherein the library members are NA building blocks which correspond to a certain region of the target polynucleotide, template or SOI. According to a specific further example, a library may be larger to comprise a diversity that allows assembly of the library members to produce a variety of ds target polynucleotides. Specifically, the diversity may even cover the entire genetic space of a species or organism. For example, a library may comprise a diversity of NA building blocks as necessary to potentially synthesize any and all naturally occurring polynucleotides of the human chromosomal genome or mitochondrial genome by assembly of the respective NA building blocks. In a further example, said diversity may cover any and all naturally occurring polynucleotides of eukaryotic species other than human, such as e.g., mouse, rat, rabbit, pig, sheep, plants, fungi or yeast. In yet another example, said diversity may cover any and all naturally occurring polynucleotides of prokaryotes, such as e.g., archaea or bacteria.

The library provided herein, specifically comprises pairs of matching NA building blocks, each consisting of two ss NA building blocks, comprising partially or fully complementary sequences. Said pair(s) of matching NA building blocks may be present in the library, wherein each of said two ss NA building blocks are comprised in separate containments, or wherein both of said two ss NA building blocks are comprised in the same containments where they may anneal and form a ds NA building block. The nucleotide sequences of a pair of matching ss NA building blocks may be complementary in at least 1, 2 or 3 nucleotides, preferably at least 4 or more nucleotides, such that a matching pair can form a new ds polynucleotide molecule by hybridization of the ss NA building block sequences, preferably wherein the ss NA building blocks hybridize in part, thereby obtaining a ds polynucleotide with an overhang.

The library preferably comprises NA building blocks which are artificially or chemically synthesized, or chemically modified (e.g., including peptidyl nucleic acids or phosphorothioate bond) NA building blocks which are synthesized by suitable methods well-known in the art. The NA building blocks comprised in the library can also be generated by enzymatic digestion of naturally occurring DNAs.

The library described herein may comprise thousands of NA building blocks as library members. Specifically, the library described herein comprises a diversity of library members, wherein each of the library members has a different nucleotide sequence.

The library described herein may specifically comprise a diversity of ds NA building block library members, wherein each of the ds NA building block library members has a different nucleotide sequence. Specifically, said diversity covers at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 750, 1.000, 2.000, 3.000, 4.000, 5.000, 10.000, 20.000, 40.000, 60.000, 80.000, 100.000, 120.000, 140.000, 160.000, 180.000 or 200.000 different ds NA building blocks or library members.

The library described herein may specifically comprise a diversity of ss NA building block library members, wherein each of the ss NA building block library members has a different nucleotide sequence. Specifically, said diversity covers at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 750, 1.000, 2.000, 3.000, 4.000, 5.000, 10.000, 20.000, 40.000, 60.000, 80.000, 100.000, 120.000, 140.000, 160.000, 180.000 or 200.000 different ss NA building blocks. Specifically, ss NA building blocks may be used as linkers, such as suitably used in the assembly of two ds NA building blocks to facilitate linkage.

Specifically, said diversity means, that different library members differ in at least one base or base pair. One library member may actually encompass multiple copies of the respective ss or ds NA building blocks, which copies consist of the same sequence. Such multiple copies of a library member are specifically contained in only one library containment.

In a specific embodiment described herein, said diversity covers NA building blocks which are modified, preferably phosphorylated.

The library described herein may comprise library members which are phosphorylated, methylated, biotinylated or which are linked to fluorophores or quenchers. As described herein, library members may comprise one or more additional phosphoryl groups.

Methylation of library members comprises the addition of a methyl group to a DNA molecule, preferably to cysteine or adenine, and is performed according to suitable DNA methylation methods well-known in the art.

As used herein, biotinylation refers to a method of covalently attaching one or more biotin molecules to a nucleic acid, such as ss or ds NA building blocks. The library members described herein may be biotinylated by suitable methods well-known in the art; preferably it is a method of chemical biotinylation. NA building blocks can be readily biotinylated in the course of synthesis by phosphoramidite methods well-known in the art, which use biotin phosphoramidite.

Library members described herein may be conjugated to a fluorophore by suitable chemical and enzymatic methods well-known in the art. Exemplary methods used for the fluorescent labeling of nucleic acids may employ a method for enzymatic labeling of DNA with fluorescent dyes e.g., using a Thermo Fisher's ARES DNA labeling kit, which employ a two-step method for enzymatic labeling of DNA with fluorescent dyes. Further exemplary methods may employ a chemical method for labeling nucleic acids without enzymatic incorporation of labeled nucleotides e.g., using a ULYSIS Nucleic Acid Labeling Kit. Further exemplary methods may employ chemical labeling of amine-terminated oligonucleotides to prepare singly labeled fluorescent oligonucleotide conjugates e.g., using an Alexa Fluor Oligonucleotide Amine Labeling Kit. Further exemplary methods may employ DNA arrays/microarrays and other hybridization techniques.

Library members may be linked to one or more quenchers, e.g., substances that absorb excitation energy from a fluorophore, by suitable methods well-known in the art. Examples of quenchers include but are not limited to Dabsyl (dimethylaminoazobenzenesulfonic acid), Black Hole Quenchers, Qxl quenchers, Iowa black FQ, Iowa black RQ and IRDye QC-1.

The library described herein specifically comprises or consists of NA building blocks which are preferably purified, may comprise modifications and are preferably kept at a standard concentration and volume in an appropriate buffer, solvent and/or excipient, e.g., ready-to-use for assembling, or be provided in a storage-stable form, such as in the dry state.

Specifically, the library members are contained in the library in a storage stable form, such as in solution or in a dry state.

Specifically, any of the following buffer and/or excipients may be used to keep the NA building blocks in solution: Tris Buffer, T.E. Buffer (Tris-EDTA Buffer) or Nuclease Free Water. Specifically, library members may be kept in Tris Buffer, wherein said Tris Buffer is provided at a concentration of about 10 mM (+/−1 mM or 2 mM). Specifically, library members may be kept in T.E. Buffer. Specifically, said T.E. Buffer is at least composed of Tris, at a concentration of about 10 mM (+/−1 mM or 2 mM), and EDTA, at a concentration of any one of 0.1, 0.2, 0.3, 0.4, 0.5, 0.6, 0.7, 0.8, 0.9 or 1.0 mM. Specifically, Nuclease Free Water, is water which has been de-ionized, filtered and autoclaved and is essentially free of contaminating non-specific endonuclease, exonuclease and RNase activity.

Specifically, any of the following solvents may be used to provide a solution of NA building blocks: ethanol, methanol, propanol, formamide, pyridine, or dimethyl sulfoxide, acetonitrile, dimethylformamide, formamide, tetrahydrofuran, MDSO, DMF, glycerol or ionic solvents, or mixtures of any of the foregoing e.g., in varying concentrations.

Specifically, all library members are kept in a compartmented array device.

In a specific embodiment, a library of NA building blocks is provided within an array device, wherein library members are contained in separate library containments.

Specifically, said array device is any of a microtiter plate, a microfluidic microplate, a set of capillaries, a microarray or a biochip, preferably a DNA and/or RNA biochip. Said array device may comprise only one, all or any type of the aforementioned containments.

Specifically, more than one different library members may be contained in only one library containment.

Specifically, said different library members contained in one library containment can be a mixture of ss NA building blocks of such sequences that are not capable of annealing to each other, thus, the ss NA building blocks are contained in the mixture as ss molecules. Specifically, said different library members contained in one library containment can be a mixture of ds and ss NA building blocks of such sequences that are not capable of assembly with or ligating to any of the other ds and ss NA building blocks contained in the mixture. Specifically, said different library members contained in one library containment can be ss and ds NA building blocks of such sequences that are not capable of assembly or annealing to one another.

The term “library diversity” or “diversity” as used herein, refers to a degree of versatility characterizing the library provided herein. Specifically, said diversity comprises aa and/or ds NA building blocks of different lengths and different sequences.

Specifically, the diversity of a NA building block library means that a variety of library members differ in at least one base or base pair. One library member may actually encompass multiple copies of ss or ds NA building blocks of the same sequence. Such multiple copies of a library member are specifically contained in only one library containment.

For example, the library may comprise all possible sequence variations of 8 nucleobase long ss oligos (herein referred to as octamers), which are 65.536 different ss oligos of 8 nucleobases length, and in addition, further ss oligos or ds oligos of different lengths, which are commonly comprised in target sequences and are, thus, used more often to build any pre-defined sequence. Including commonly used ss or ds NA building blocks into a library's diversity decreases synthesis cost and increases time efficiency.

Specifically, said diversity may cover an entire genome, for example the human genome. Specifically, said diversity may cover the entire genetic space. Specifically, said diversity may cover a genome or the entire genetic space multiple times in multiple ways. For example, the diversity may encompass all possible hexamer, heptamer and/or octamer sequence combinations, or even encompass all or selected 9-mers, 10-mers and/or up to 26-mers.

Each library member may be individually characterized and marked by a selectable marker or a DNA sequence tag or barcode, to facilitate the selection of a library member in the library or the identification of a library member in the library. Alternatively, the genetic mutation may be determined directly by a suitable determination method e.g., high-throughput sequencing, capillary sequencing or employing specific probes hybridizing with a predefined sequence, to select the corresponding oligonucleotide.

It may be desirable to place each of the diverse library members in separate containers, to obtain a library of NA building blocks in separate containers. According to a specific embodiment, the library is provided in an array e.g., a DNA biochip, wherein the array comprises a series of containments or spots on a solid carrier.

The term “sequence of interest” or “SOI” as used herein refers to the desired nucleotide or base pair sequence of the ds target polynucleotide which is to be produced by the method provided herein.

The term “solid support” as used herein is understood as a solid material often used in oligo or polynucleotide synthesis because it allows easy washing and cleanup of a NA reaction mixture from other chemicals, enzymes, unreacted oligos, etc. By keeping a NA assembly attached to a substrate, there is more control on the assembly. However, the yield tends to be lower than in solutions posing other challenges. Nevertheless, use of solid-support attachment for enzymatic and/or chemical assembly may be useful e.g., for purification or amplification steps. In general, the purpose for using a solid support-attached oligo or polynucleotide is to capture something, hold on to it, enrich it, and identify and/or purify it.

Examples of common solid supports include microarrays or beads. The most generally accessible approach for producing NA building block microarrays is to synthesize the individual NA building blocks separately prior to immobilization on the solid surface. In this case, the NA building block can be modified with a functional group that allows attachment to a reactive group on a solid surface. Another important class is the micro-bead-attached NA building block. Some modifications include: amine-NA building blocks covalently linked to an activated carboxylate group or succinimidyl ester, SH-NA building blocks covalently linked via an alkylating reagent such as an iodoacetamide or maleimide, acrydite-NA building blocks covalently linked through a thioether, and biotin-NA building blocks captured by immobilized Streptavidin.

Specific methods include photo-cleavable chemical moieties that allow detachment from a solid support by irradiation. WO2019140353A1 discloses a method where oligos are attached through an anchor oligonucleotide that contains an uracil base. By treating with uracil-N-deglycosylase (UNG) abasic nucleotide is left in place of the uracil. Then, treating the sample with either Endo IV or APE-I, which are endonucleases capable of cutting at a single stranded abasic site, the fragments are released form the surface.

The term “target double stranded (ds) polynucleotide” refers to a polynucleotide having a predefined sequence, which is produced by the method provided herein. Specifically, said target ds polynucleotide is characterized by a sequence which is identical and/or corresponding to a SOI. If the target ds polynucleotide sequence has a sequence which is less than 100% identical to a SOI, the target ds polynucleotide is understood as a proxy ds polynucleotide that can be further modified to produce a ds polynucleotide that has a sequence which is identical and/or corresponding to the SOI.

The foregoing description will be more fully understood with reference to the following examples. Such examples are, however, merely representative of methods of practicing one or more embodiments of the present invention and should not be read as limiting the scope of invention.

### EXAMPLES

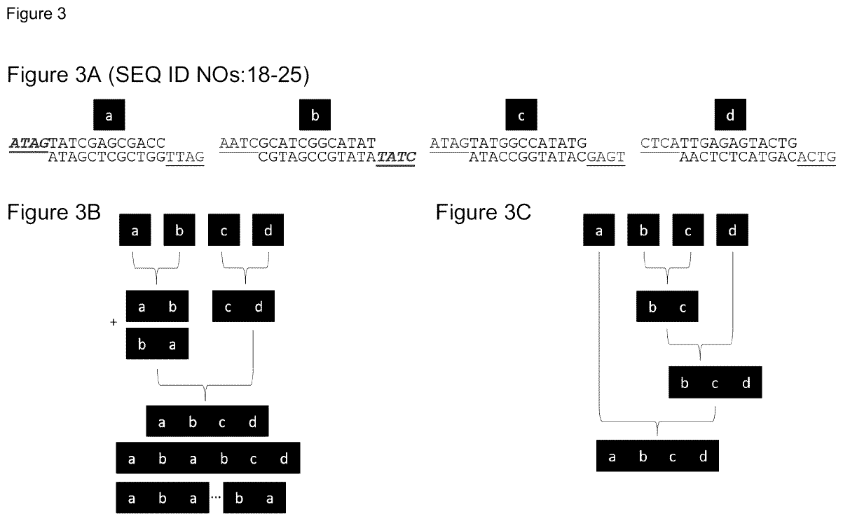

The methods disclosed herein refer to a hierarchical assembly, albeit not a symmetric one. Instead of reacting all oligos or reaction products at every tier of the assembly, only subsets of these are assembled at any given tier, with the remaining ones assembled at later tiers, achieving an asymmetric assembly. In such a way, ambiguous assemblies are avoided, thereby successfully completing the desired product which cannot be otherwise completed with a symmetric assembly (FIG. 3).

For example, to assemble oligos a,b,c and d into the construct abcd, a symmetrical assembly workflow would be: in a first tier oligos a+b and c+d are reacted in two independent compartments to give first reaction products ab and cd. In a second reaction these two products are reacted in a compartment to yield the desired abcd polynucleotide. This assembly workflow can be represented as a binary tree in bracket notation {{a,b},{c,d}}.

According to a specific example, a and b are assembled by ligation with sticky-ends and the sticky ends permit not only ab as a reaction product but also ba as a second (undesired) reaction product (FIG. 3). Necessarily, in this particular case, other reaction products such as (ab)ₙ, (ba)ₙ would also form, impeding a successful assembly at the second tier.

This problem can be circumvented using two convoluted principles:
(i) a method of partitioning the sequence into sub-sequences, and
(ii) a method for determining an assembly workflow.

Partitioning is the process of determining a set of sub-sequences of oligonucleotides or polynucleotides in such a way that when processed with an appropriate assembly workflow they result in an assembled polynucleotide with the sequence of interest.

The assembly workflow is the series of hierarchical steps that are carried out to achieve that assembly. For instance, the assembly workflow above {{a,b},{c,d}} is the simplest hierarchical workflow possible. But others can be more efficient, depending on the sequence.

According to this example, the method that is disclosed herein comprises three aspects:
(i) a partitioning of the sequence, preferably using an algorithm, in a way that it eliminates as many non-specific assembly steps as possible;
(ii) a sequence-specific assembly workflow that ensures that no ambiguities remain and
(iii) the actual enzymatic or chemical assembly of the oligo/polynucleotides starting from the sequences defined in (i) following the steps defined in (ii) into the target sequence.

More specifically, the partitioning of a target sequence into smaller polynucleotides or oligonucleotides can be done in a way where these building blocks have different lengths, but equal overhang length, or where they have different overhang lengths, and then, when assembled in non-trivial assembly workflows that are sequence-dependent and non-symmetric (or asymmetric), as explained below, a successful synthesis of a target polynucleotide is achieved in a way that cannot be done with naïve partitions that do not consider the mis-assembly risk and/or hierarchical but sequence-independent symmetric workflows.

In the following subsections the partitioning and the assembly workflows are described in more detail.

### A. Asymmetric Assembly Workflows.

In some cases, for instance, if the components (oligos) are derived from restriction digests, there may not be ways to redesign the oligos as indicated above. Thus, one cannot rely on oligo design alone. To overcome this problem, the way in which the oligos are assembled and an asymmetric assembly strategy was developed.

In the strategy of asymmetric assembly trees, in a first reaction b and c can be assembled into bc, then this reaction product can be reacted with, d to give bcd and only after, in a third tier react the last product with a to obtain abcd (FIG. 3b). This assembly can be represented by a binary tree {a,{{b,c},d}}. Note that also other alternative assemblies could potentially work, such as {{a,{b,c}},d} or {{a,{b,{c,d}}} (FIG. 3c). However, the number of possible assembly trees for a given number of starting oligos N is combinatorically big, thus, the problem arises of deciding which of these is sufficient in order to successfully complete an assembly. For longer target sequences it is thus beneficial to use a computational method as a first step.

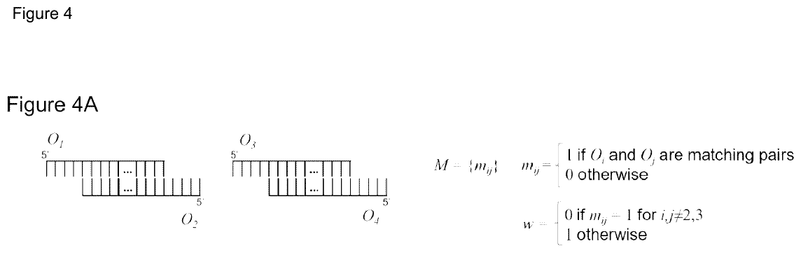

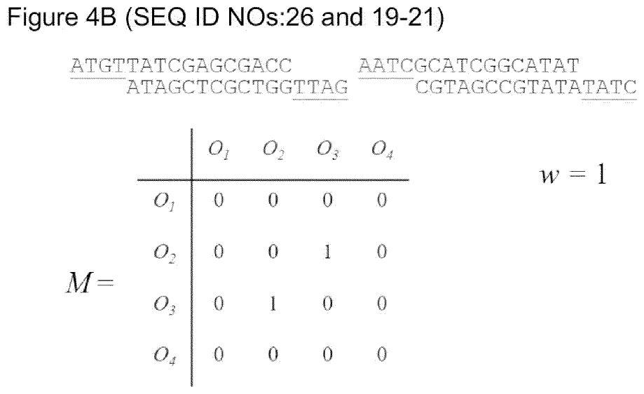

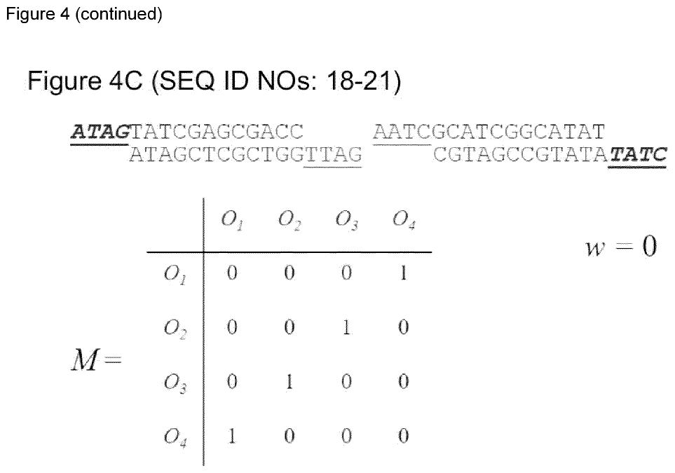

First, a measure for the uniqueness (or not uniqueness) of the assembly of two oligos is assigned, in order to verify their validity in the assembly (examples given below). This measure can be assigned to every node in the tree and, then, all nodes' measures convolved by, for example, multiplying the weights at each node, to give a single measure to the complete workflow in a way that correlates with the effectiveness of the actual assembly and/or to the success probability of the assembly (FIG. 4).

By using a measure with these properties, it is then possible to identify an optimal, or nearly so, assembly workflow by performing some search in the space of all potential trees with an optimization method or selection criteria.

Once an optimal or nearly optimal workflow has been identified, this workflow can be employed in order to complete the assembly e.g., by means of liquid handlers or microfluidic devices. Considering that there are many possible assembly workflows, the measures in question can consider also as a second criterion such as minimizing the number of tiers or to have the most symmetric assembly possible that does not lead to failure.

B. Modification of Oligo Length with Equal Oligo Size

Where there are oligos that have self-complementary overhangs or that are able to self-ligate (e.g., with overhangs like ATAT, TATA, CGCG, GCGC or variations that allow complementarity with wobble pairs, Hoogsteen pairs or by any other criterion that indicates that the oligos can self-ligate), then the partition is deemed invalid. In this case, the problematic oligonucleotide can be re-designed together with the immediate pair so that they are slightly larger or shorter, until the ambiguity is resolved. For example, consider the two oligo dimers:

| Oligo a | Oligo b |
|---|---|
| ...TCGAGGAACGC | ATATCGGTAAA... |
| ...AGCTCCTTGCGTATA | GCGATTT... |

Oligo sequences referred to above, regions that are complementary to each other are underlined:

| | Oligo a, leading strand: |
|---|---|
| (SEQ ID NO: 61) | |
| | TCGAGGAACGC |
| | Oligo a, lagging strand: |
| (SEQ ID NO: 62) | |
| | ATATGCGTTCCTCGA |
| | Oligo b, leading strand: |
| (SEQ ID NO: 63) | |
| | ATATCGGTAAA |
| | Oligo b, lagging strand: |
| (SEQ ID NO: 64) | |
| | TTTAGCG |

Both, Oligo a and Oligo B will be self-ligating because the overhang is ambiguous. The oligo design can be modified by transferring the 3′C and 5′A of Oligo a to the 5′ and 3′ ends, respectively, of Oligo b.

| Oligo a′ | Oligo b′ |
|---|---|
| ...TCGAGGAACG | CATATCGGTAAA... |
| ...AGCTCCTTGCGTAT | AGCGATTT... |

Oligo sequences referred to above, regions that are complementary to each other are underlined, transferred nucleosides are bold and underlined:

| | Oligo a′, leading strand: |
|---|---|
| (SEQ ID NO: 65) | |
| | TCGAGGAACG |
| | Oligo a′, lagging strand: |
| (SEQ ID NO: 66) | |
| | TATGCGTTCCTCGA |
| | Oligo b′, leading strand: |
| (SEQ ID NO: 67) | |
| | CATATCGGTAAA |
| | Oligo b′, lagging strand: |
| (SEQ ID NO: 68) | |
| | TTTAGCGA |

Now, Oligo a′ is one base shorter than Oligo a and Oligo b′ is one base longer than Oligo b, which is an asymmetric pair of oligos, and, importantly, they do not self-ligate anymore.

This strategy can be employed to find more efficient partitions even if the oligos are not self-ligating.

### C. Modification of Overhang Length

The problem of self-ligation or any other ambiguity in the ligation can be solved also by a modification of the length of the overhang. Considering the same example as above, namely the two self-ligating oligos that appear contiguously in the sequence:

| Oligo a | Oligo b |
|---|---|
| ...TCGAGGAACGC | ATATCGGTAAA... |
| ...AGCTCCTTGCGTATA | GCGATTT... |

Oligo sequences referred to above, regions that are complementary to each other are underlined:

| | Oligo a, leading strand: |
|---|---|
| (SEQ ID NO: 61) | |
| | TCGAGGAACGC |
| | Oligo a, lagging strand: |
| (SEQ ID NO: 62) | |
| | ATATGCGTTCCTCGA |
| | Oligo b, leading strand: |
| (SEQ ID NO: 63) | |
| | ATATCGGTAAA |
| | Oligo b, lagging strand: |
| (SEQ ID NO: 64) | |
| | TTTAGCG |

can be partitioned in a different manner so that one base of one oligo sequence in the naïve partition is instead included in the other oligo, as for example:

| Oligo a′′ | Oligo b′′ |
|---|---|
| ...TCGAGGAACGCA | TATCGGTAAA... |
| ...AGCTCCTTGCGTATA | GCGATTT... |

Oligo sequences referred to above, regions that are complementary to each other are underlined, transferred nucleosides are bold and underlined:

| | Oligo a″, leading strand: |
|---|---|
| (SEQ ID NO: 69) | |
| | TCGAGGAACGCA |
| | Oligo a″, lagging strand: |
| (SEQ ID NO: 62) | |
| | ATATGCGTTCCTCGA |
| | Oligo b″, leading strand: |
| (SEQ ID NO: 70) | |
| | TATCGGTAAA |
| | Oligo b″, lagging strand: |
| (SEQ ID NO: 64) | |
| | TTTAGCG |

or, even in a manner that increases the oligo overhang length, i.e.

| Oligo a′′′ | Oligo b′′′ |
|---|---|
| ...TCGAGGAACG | CATATCGGTAAA... |
| ...AGCTCCTTGCGTATA | GCGATTT... |

Oligo sequences referred to above, regions that are complementary to each other are underlined, transferred nucleosides are bold and underlined:

| | Oligo a′′′, leading strand: |
|---|---|
| (SEQ ID NO: 71) | |
| | TCGAGGAACG |
| | Oligo a′′′, lagging strand: |
| (SEQ ID NO: 62) | |
| | ATATGCGTTCCTCGA |
| | Oligo b′′′, leading strand: |
| (SEQ ID NO: 72) | |
| | CATATCGGTAAA |
| | Oligo b′′′, lagging strand: |
| (SEQ ID NO: 64) | |
| | TTTAGCG |

### D. Computation of the Assembly Workflow.

A given target Sequence of Interest (SOI) is processed by the algorithm to provide (a) a list of ss oligonucleotide sequences (oligos) and (b) the workflow W to assemble those sequences. Note that oligo sequences and W may be different depending on the assembly method (see below). Also, depending on the nature of the algorithm that is used a different output can be produced although of similar efficiency of assembly. Different criteria can be chosen to partition the sequence, as for example, computational parameters that consider Watson-Crick and wobble and/or Hoogsteen base pairs, a percentage of mismatch, enzyme kinetic parameters, annealing thermodynamics, indices for ligation/cloning efficiency, physico-chemical, statistical or phenomenological formulas, or algorithmic processes. Note that there are sequences that cannot be partitioned (or assembled), such as (AT)ₙ (TA)ₙ (CG)ₙ (GC)ₙ and other extreme cases of high regularity.

### 1. Provision of the Oligonucleotides or Polynucleotide Fragments.

Oligonucleotides may be provided as a library in array format in micro-well plate, procured from any provider or made using phosphoramidite synthesis, TdT enzymatic or any other method. They could also be directly synthesized in the microfluidic chip ready for assembly. However, it may be needed that these are annealed, if a ligation-based assembly will be used. If a PCR-based assembly is used instead, they may be provided and used directly as single stranded oligos. The oligos or polynucleotides may have any length.

### 2. Assembly of the Polynucleotides.

The method can be used to assemble short oligos (e.g., 8 nt) into polynucleotides by ligation with sticky ends, by Gibson Assembly (GA), considering that the different oligos have sufficient overlap, and where at any tier a ligation or GA reaction is incubated by providing the necessary enzymes, or gene fragments assembled by golden gate. Oligos may be attached to solid substrate (e.g., avidin attachment through biotin modifications or any other method) in magnetic beads or coated surfaces, thereby increasing accuracy and facilitating purification steps in between assembly tiers. Implementation of the asymmetric assembly steps can be done by using liquid transfers (by automated handlers or microfluidic devices).

### 3. Postprocessing of the Polynucleotides.

This can include purification, amplification by means of high-quality PCR, bacterial cloning, or any other process required to finalize and enrich the assembled molecule that has the SOI.

### Example 1. Algorithm for Partitioning the Sequences and Computing the Tree

### Choosing the Objective Function

An objective function (score) was chosen for evaluating a partition by defining a weight to every node n according to whether the oligos at each node can react in a unique manner (w=1) or not (w=0). More specifically, any two oligos have a total of four overhangs (O₁, . . . , O₄) (see FIG. 4a for numbering of the overhangs) where we want that
O₂ and O₃ match according to Watson-Crick pairing
any other pair of overhangs does not match, also excluding “wobble” or Hoogsteen pair variations.

Mathematically, this can be represented by a symmetric adjacency matrix M (Mᵢⱼ=Mⱼᵢ) where a 1 indicates a matching pair and a 0 indicates the pair does not match. The score wₙ at node n is then defined as

$$w = \{ \begin{matrix}0 & if {O}_{ij} = 1 for i , j \neq 2 , 3 \\ 1 & otherwise\end{matrix}$$

For example, FIG. 4b shows a pair of oligos that match in a unique manner, so only the element M_{2,3} whereas FIG. 4c, aside from having the desired matching O₁ and O₄ also match which would lead to a ligation in an undesired manner. Therefore, the elements

M_{1,4}=1 and therefore w=0.

As indicated above, other metrics can be incorporated based, for example, in relation to ligation activity, on frequency of recovered clones from a controlled experiment with proper design of overhang pairs. Metrics need not to be binary, but were chosen to be in this example for simplicity and clarity (see Example 5).

### Partitioning the Sequence

SEQ ID NO:1 was chosen as SOI. In this example, it was first partitioned in a naïve manner, that is in equal lengths of 16 nt and with overhangs of length=4. This results in 16 single stranded oligonucleotide sequences that, once the target double stranded polynucleotide is assembled, will be identical in sequence to the SOI.

### Symmetric and Asymmetric Assembly Trees

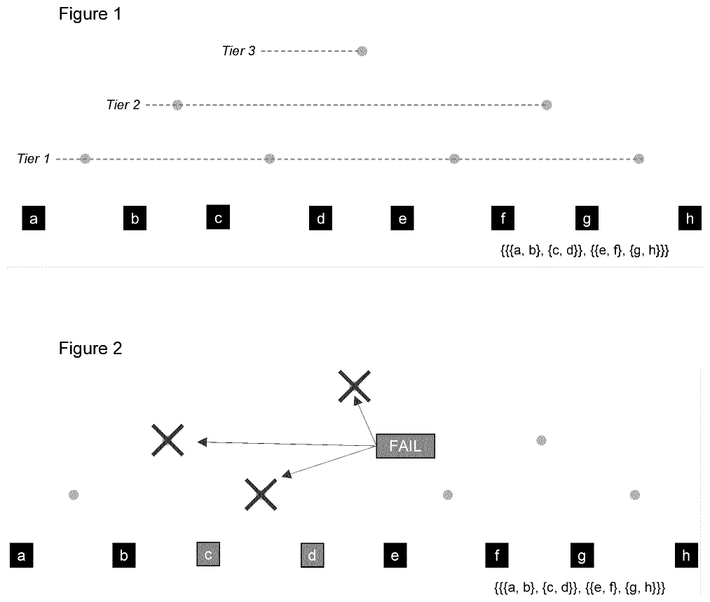

From the resulting selection of oligos we compare two partitions: a symmetric and an asymmetric one. The former is, as disclosed in the prior art, the simplest binary tree (FIG. 1). To choose an asymmetric tree the following steps were applied:
i. Start with a symmetric tree
ii. Evaluate the score at each node
iii. If there are nodes that have score w=0, the branches are modified by re-splitting at one deeper node in such a way that the conflicting oligos are not in the same immediate node
iv. Repeat steps ii-iii 1 million times until there is a solution
v. If there is no solution after 1 million trials, the sequence is re-partitioned and the assembly tree computed by starting at step i.

Other approaches can be used, especially when sequences are longer and not all assembly trees can be computed due to combinatorial complexity; for example, by Monte Carlo sampling a set of assembly trees that are valid as measured by the metric above followed by picking a valid tree. This can result in asymmetric assembly workflows represented by trees that are “unbalanced” (in the technical graph jargon) and that lead to successful assemblies.

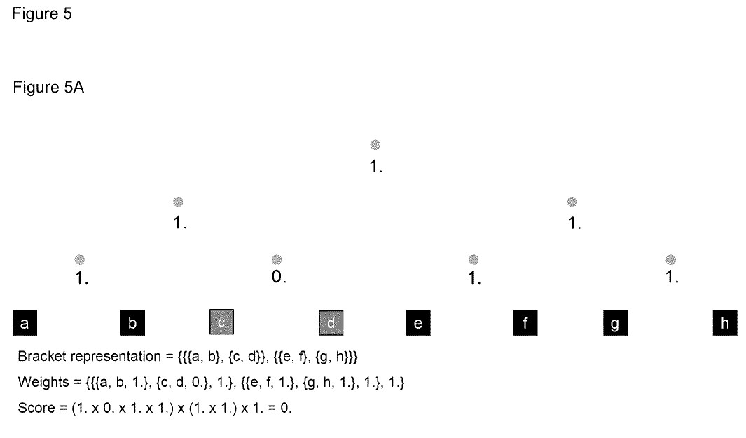

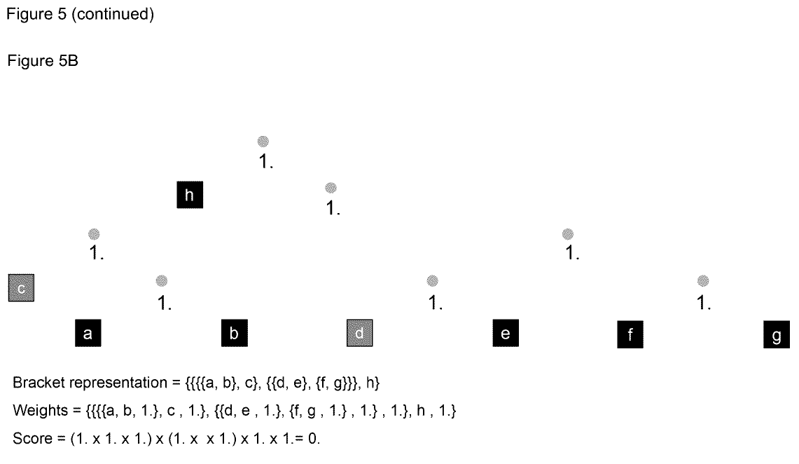

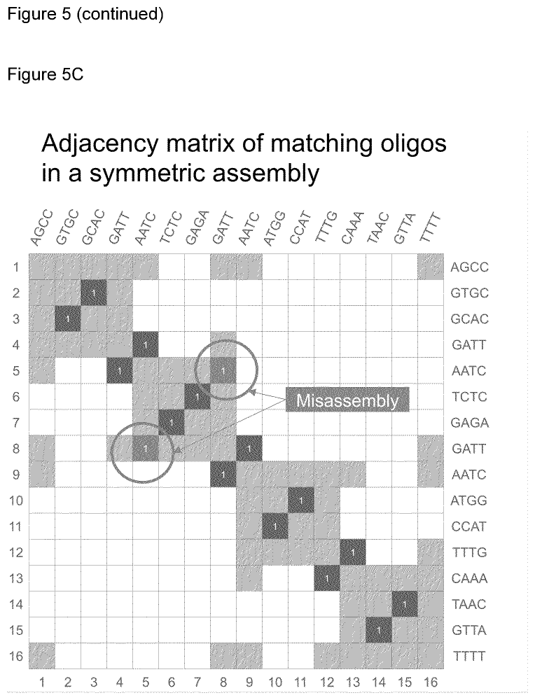

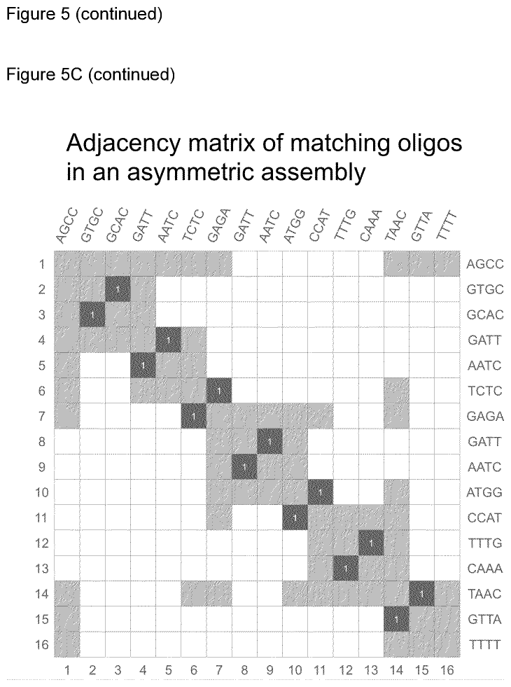

FIG. 5 shows the result of a flat, naïve partition (symmetric) that presents one problematic node in oligos c and d and an asymmetric assembly tree that resolves the conflict. FIG. 5c shows the adjacency matrices of the problematic node (oligos matching in an undesired manner) revealing that under the symmetric assembly (left matrix) workflow there are anticipated assembly problems, whereas under the asymmetric one (right matrix), there are none. The adjacency matrices consider not only the overhang matching but also the local adjacency as determined by the assembly workflow.

In bracket notation, the result of the partition of the asymmetric assembly tree is represented as {{{{a, b}, c}, {{d, e}, {f, g}}}, h}, which in graph representation is the tree of FIG. 5b.

### Example 2. Assembly of a Target Molecule of 128 bp with an Asymmetric Workflow and Comparison to a Symmetric Workflow by Ligation of 16-Mers

After partitioning the target sequence (SEQ ID NO:1) and defining the assembly trees as described in Example 1, the oligos are assembled by following both a symmetric assembly (FIG. 5a), and by following the asymmetric tree described above (FIG. 5b), namely {{{{a, b}, c}, {{d, e}, {f, g}}}, h}.

All oligos a-h were procured by Integrated DNA Technologies (IDT) with standard desalting purification and were provided normalized at a concentration of 50 UM on IDTE Buffer (pH 7.5). The oligos used in the assemblies below were single-stranded and pure.

### Preparing the Annealing Solutions and Annealing

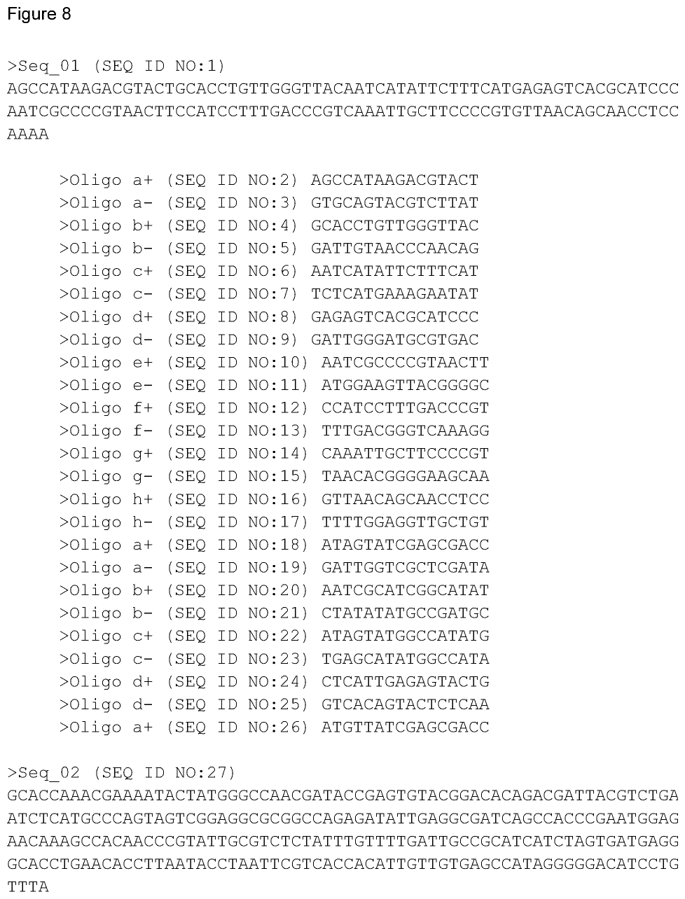

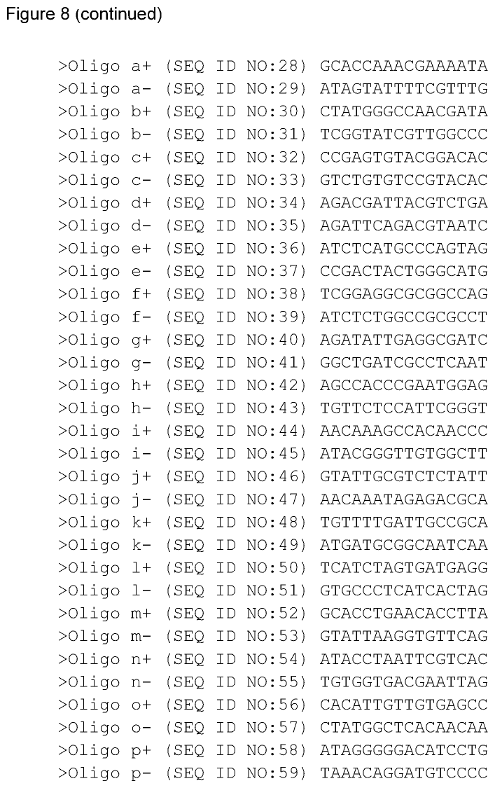

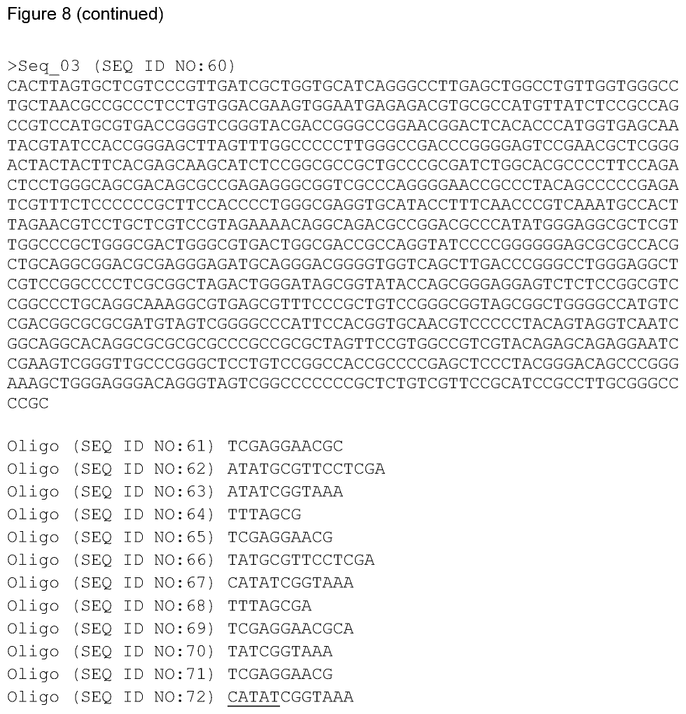

Some commercial buffers are ready to mix in H₂O such as New England Biolabs' (NEB) T4 Ligase Reaction Buffer, product nr B0202S, and readily contain the ATP necessary for the ligase activity. In a microcentrifuge tube, 216 μl of ddH₂O with NEB T4 ligase buffer were prepared and the solution was mixed well by vortexing. 24 μL of this solution mix were dispensed into to 8 reaction tubes labelled a-h. 3 μL of each oligo was transferred to 8 predefined tubes and mixed well by pipetting (sequences see FIG. 8, SEQ ID NOs: 2 to 17):
Oligos a− and a+ into tube a
Oligos b− and b+ into tube b
Oligos c− and c+ into tube c
Oligos d− and d+ into tube d
Oligos e− and e+ into tube e
Oligos f− and f+ into tube f
Oligos g− and g+ into tube h
Oligos h− and h+ into tube h

The tubes were sealed and incubated in a thermocycler for 30 sec at 98° C. The temperature was then decreased from 95° C. to 24° C. with a ramp function that diminished the temperature by 1° C. per minute allowing the matching pairs of ss oligos to anneal. Once finished the double stranded oligos were kept at 4° C.

### Preparing the Ligation Solution.

The ligation solution was prepared on ice by mixing, in the following order, 32.5 μL of nuclease free ddH2O, 7.5 μL of ligase buffer and 5 μL of ATP for a final concentration of 10 mM. The ligation solution was mixed well by vortexing and spun down. 2.5 μL of T4 Ligase (NEB, product nr. M0202) & 2.5 μL of T4 polynucleotide kinase (PNK) (NEB, product nr. M0201B) were added for a total of 10 and 0.25 units per μL of final solution, respectively, and mixed well by gently pipetting. The solution was kept on ice until needed. 10 μL of the ligation solution were transferred to each of 4 tubes (b, d, f, h) containing 5 μL the corresponding ds oligos and mixed by pipetting. Afterwards the tubes were sealed again.

### Symmetric Assembly

The oligos are arrayed in rows on a 96-micro-well plate and pairs of oligos or reaction products are transferred in tiers (see FIG. 1):
i. First assembly tier. Transfer the contents of oligo mixtures a into mixture of oligo b (plus ligation solution), of oligo mixtures c into mixture of oligo d (plus ligation solution), of oligo mixtures e into mixture of oligo f (plus ligation solution), and of oligo mixtures g into mixture of oligo h (plus ligation solution).
ii. After gently mixing by pipetting, these two reactions were incubated for 30 min at 24° C.
iii. Second assembly tier. Transfer the contents of oligo mixtures ab (tube b) into mixture of oligo cd (tube d) and of oligo mixtures ef (tube f) into mixture of oligo gh (tube h).
iv. After gently mixing by pipetting, these two reactions were incubated for 30 min at 24° C.
v. Third assembly tier. Transfer the contents of oligo mixtures a.d (tube d) into mixture of oligo e.h (tube h).
vi. After gently mixing by pipetting, these two reactions were incubated for 30 min at 24° C.
vii. The final volume containing the 128 bp product was 80 μL.
viii. Heat inactivating the ligation reaction by incubating for 10 min at 65° C.

### Asymmetric Assembly by Step-by-Step Hand-Pipetting

In this case, it suffices to keep track of the order in which the oligos and their reaction products are combined, by proceeding according to the tree in FIG. 5b. The reaction conditions are the same as in the symmetric assembly. Although the asymmetric assembly will follow the steps in a similar manner as with the symmetric assembly, it is important to notice that the order of the transfers is crucial.
i. First assembly tier. Transfer the contents of oligo mixtures a (tube a) into mixture b (tube b), oligo mixtures d (tube d) into mixture e (tube e) and oligo mixtures f (tube f) into mixture g (tube g).
ii. After gently mixing by pipetting these two reactions were incubated for 30 min at 24° C.
iii. Second assembly tier. Transfer the contents of oligo mixtures c (tube c) into mixture a.b (tube b), the contents of oligo mixture d.e (tube e) into mixture f.g (tube g).
iv. After gently mixing by pipetting these two reactions were incubated for 30 min at 24° C.
v. Third assembly tier. Combine the reaction product a.g (tube g) with the mixture h (tube h).
vi. Gently mix by pipetting and incubate the reaction for 30 min at room temperature.
vii. The final volume containing the 128 bp product was 70 μL.
viii. Heat inactivating the ligation reaction by incubating for 10 min at 65° C.
ix. Optionally, perform a bead purification by using MagMax beads of the final product following the provider's protocol. Alternatively, separation on agarose gel can be performed and the DNA extracted using purification kits, such as the Monarch PCR & DNA clean up kit from New England Biolabs (product nr. T1030).

### Example 3. Assembly of a Target Molecule of 128 bp (SEQ ID NO:1) with an Asymmetric Workflow by Ligation of 16-Mers Using Liquid Handlers

In order to adapt the method to the use of automated liquid handlers, the order of the oligos is re-arranged and then liquid-handling steps are performed. Although this seems unnecessarily complicated for a set of 8 oligo dimers, longer sequences will require hundreds of dimers arrayed in a complex manner making the process highly prone to error (see Example 4). The detailed step-by-step instructions as described above may be confusing, in particular because the order of steps will vary according to the target sequence. For this reason, an alternative was developed, which is based on a scheme where the oligos are initially arrayed in a specific manner (also sequence dependent).

### Arraying the Oligos

To facilitate the process of assembly by means of liquid handlers (multichannel pipettes or preferably automatic liquid handlers), a particular arraying of the oligos can be done that directly maps the results of the assembly tree to non-trivial placement of oligonucleotides on a microwell plate.

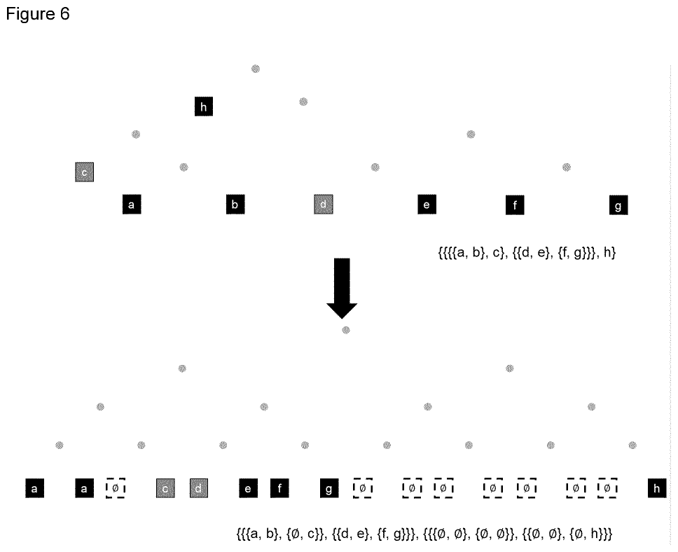

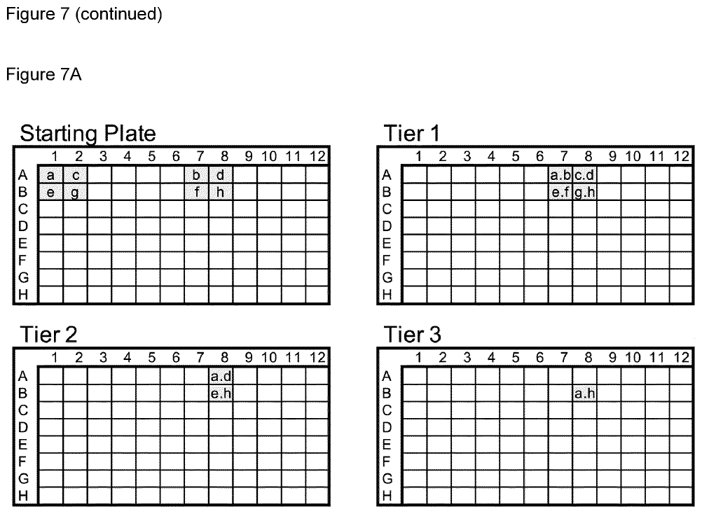

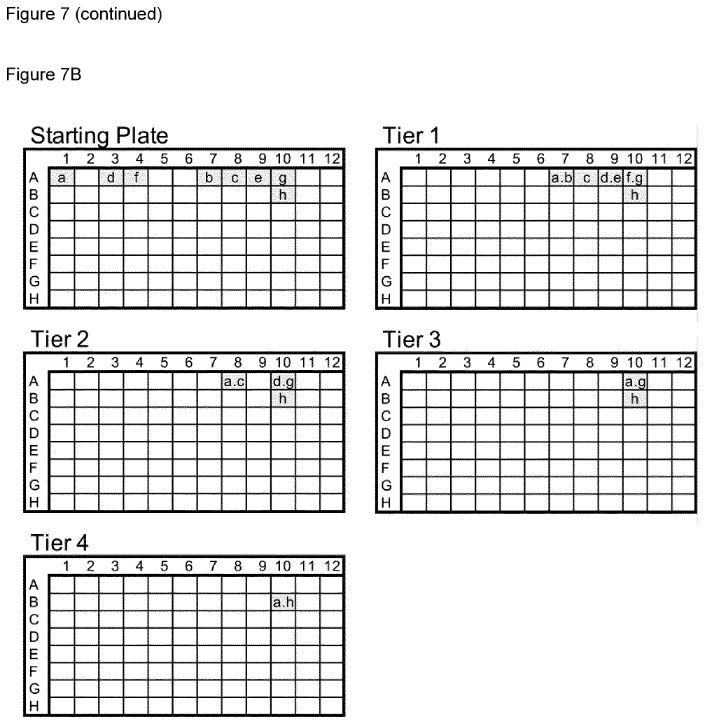

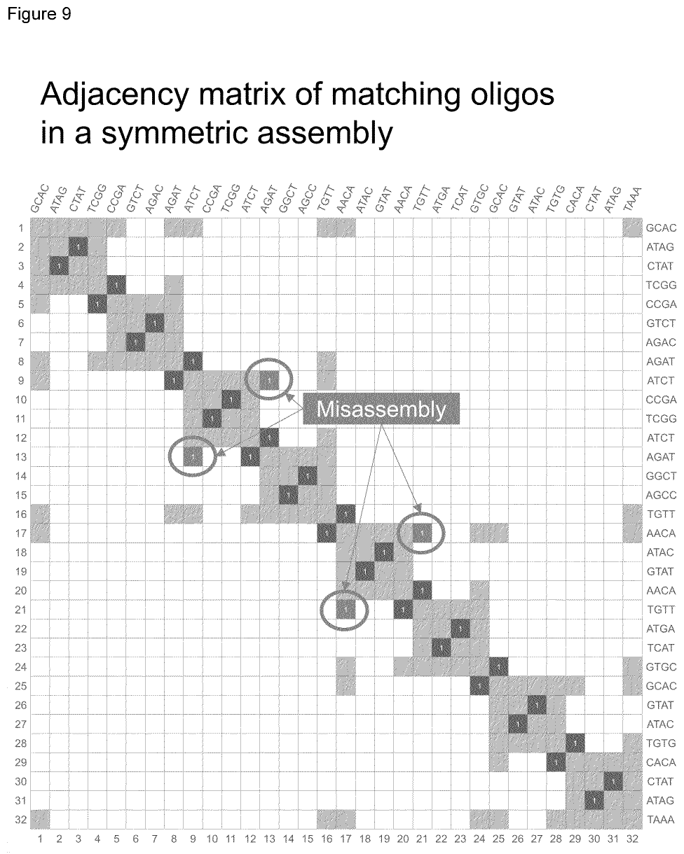

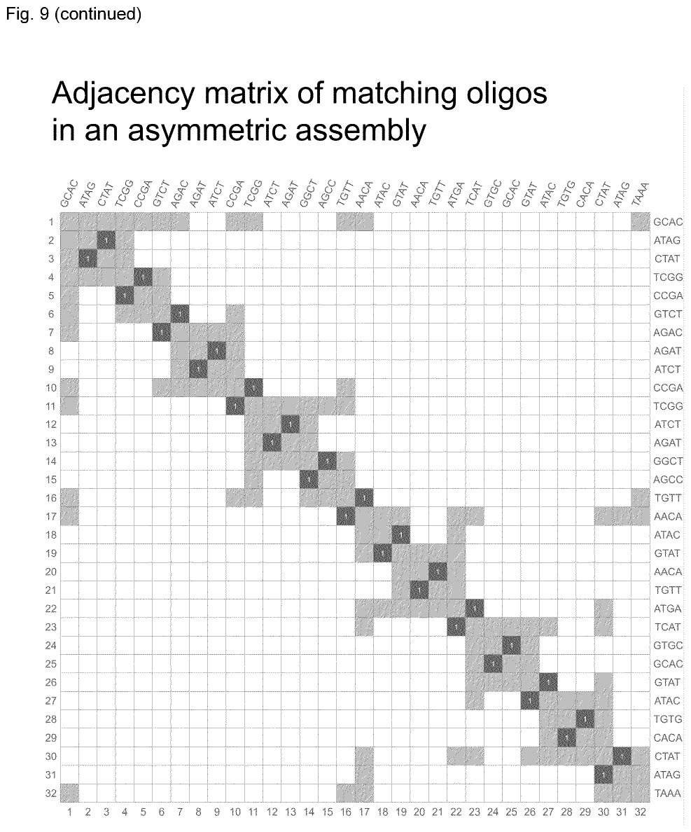

The asymmetric assembly tree can be casted into a larger tree of the same depth (nr. of tiers) that is symmetric but has empty leaves, or “Zeromers”, denoted by ø (FIG. 6). Note that the zeromers are only an abstraction; in an experimental set-up they will correspond to empty wells or solution without oligos. In this example, the zeromers are always introduced to the left of any oligo (this has to do with mechanical transfer reasons, namely, to avoid moving liquids unnecessarily, however the same result can be achieved by inverting the direction of the transfers and the position of the zeromers) or sub-tree. In the case of the 8 oligos, the smallest symmetric tree is one having a total of 16 leaves containing 8 zeromers. FIG. 7a shows how the 8 oligos would be arrayed to perform regular transfers in a symmetric assembly workflow. FIG. 9b shows how the tree with zeromers is arrayed in a non-trivial manner, which will facilitate transfer by a skilled person or by a liquid handler without worrying about the contents or specific movements of the wells. This is particularly important for longer sequences. For clarity and didactic reasons, however; it is described herein using a shorter sequence.

### Procuring the Oligos

The arraying is a 1-to-1 mapping from the assembly tree to a 96 (or 384) micro-well plate. Thus, these can be directly procured or provided with the necessary array format.

In the case of the asymmetric assembly tree {{{{a, b}, c}, {{d, e}, {f, g}}}, h} the oligos are arrayed as in the Starting Plate of FIG. 7, which is according to the following table 1:

TABLE 1
Oligo Array

Micro-well

| | Symmetric | Asymmetric |
|---|---|---|
| Oligo | Assembly | assembly |
| a | A1 | A1 |
| b | A7 | A7 |
| c | A2 | A8 |
| d | A8 | A3 |
| e | B1 | A9 |
| f | B7 | A4 |
| g | B2 | A10 |
| h | B8 | B10 |

### Assembling the Oligos

Arrayed in this manner, both the symmetric and asymmetric assembly trees can be implemented by regular movements. In the case of asymmetric trees, as indicated above, there are always one or more tiers of assembly. Yet the movements of the handler can be regular, even if they sometimes include some actions that do not carry liquids (e.g. wells A5-A6 or well B1-B9).

### Example 4. Assembly of a Target Molecule of 256 bp with an Asymmetric Workflow by Ligation of Oligonucleotides of 16 bp and Comparing to Symmetric Assembly

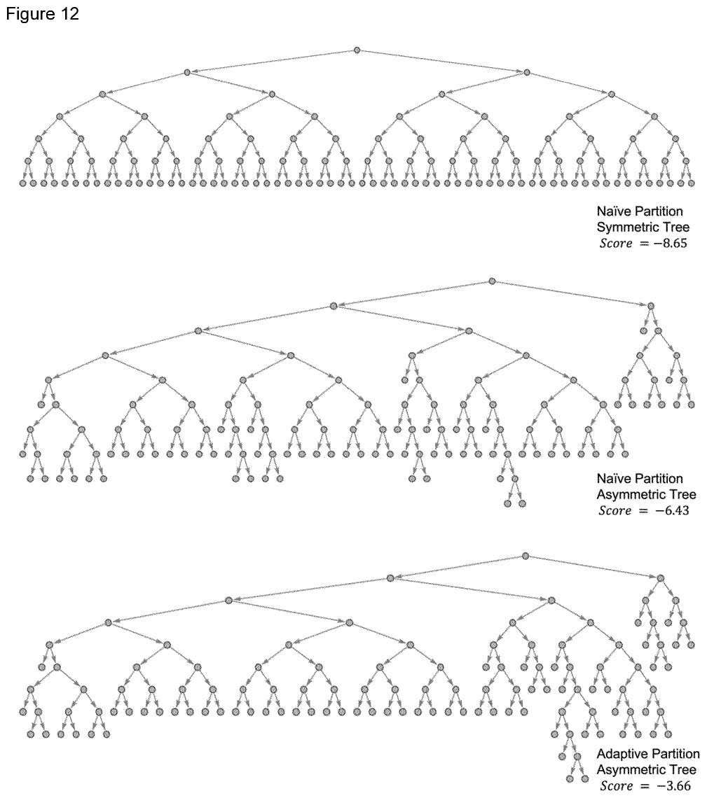

In this example, a 256 bp sequence was used (SEQ ID NO:27). After partitioning the target sequence and defining the assembly trees as described in Example 1. The adjacency matrices (FIG. 9) show two problematic nodes in the symmetric assembly. The oligos are assembled by following both a symmetric assembly (FIG. 12a), and by following the asymmetric tree described above (FIG. 12b), namely {{{{{a, b}, c}, {d, e}}, {{f, g}, h}}, {{{i, {j, k}}, {{l, m}, {n, o}}}, p}}.

### Asymmetric Assembly by Step-by-Step Hand-Pipetting

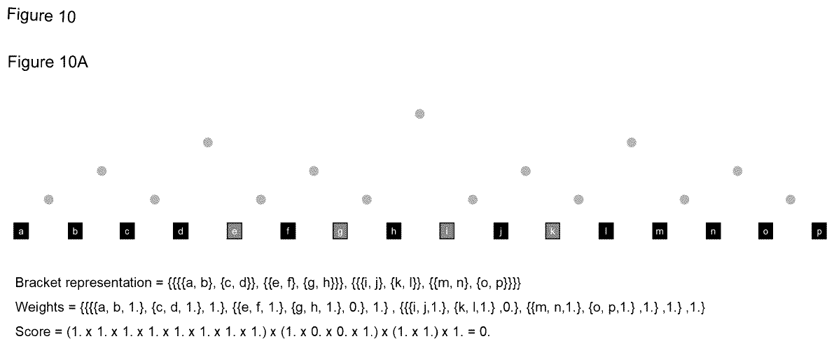

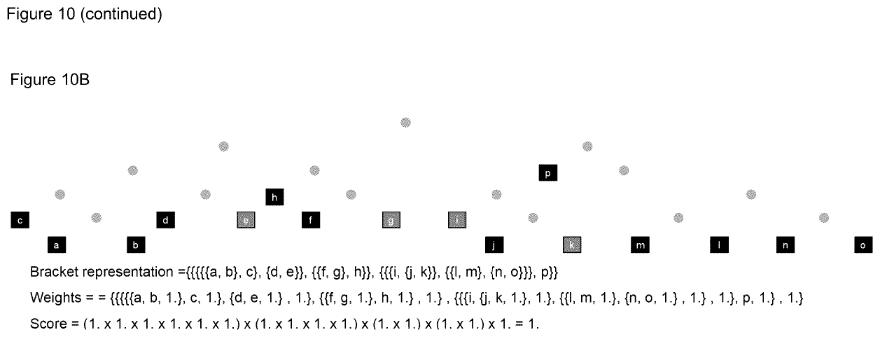

The assembly procedure is analogous to the one described in Example 2, but this time the number of required steps is larger to account for a larger assembly tree. Specifically, steps i-viii. follow a similar pattern as in the aforementioned example (with a different combination of mixing steps according to the different assembly tree), for the two main branches of the tree (FIG. 10b). At this point we have tube h containing mixture a.h and tube p containing mixture i.p. We proceed as follows:
ix. A bead purification of 128 bp products was performed by using MagMax bead technology and following manufacturer's instructions with the following modifications to the protocol: binding solution buffer to sample ratio used was 1.66. After beads purification, DNA was eluted in 17 μl of ligation mix (T7 ligase (75 U/μl), T4 PNK (0.25 U/μl), 10 mM ATP, 10×T4 NEB Buffer and H₂O)
x. Fourth assembly tier. Combine the reaction product a.h (tube h) with the mixture i.p (tube p).
xi. Gently mix by pipetting and incubate the reaction for 60 min at 24 C.
xii. A bead purification of final 256 bp products was performed by using MagMax beads following the manufacturer's protocol with the following modifications to the protocol: binding solution buffer to sample ratio used was 1, elution in 17 μl ddH₂O.

### Visualization and Comparison of the Results

Following ligations, final products were subjected to quality control using capillary electrophoresis (Fragment Analyzer). Prior to the loading of the samples on the FA, samples were diluted 2× in 1×TE Buffer. Samples were run on the FA using Small Fragment Kit (Agilent, DNF-476-0500) and using corresponding SS Small Fragment method (Agilent, DNF-476-33) following manufacturer's instructions.

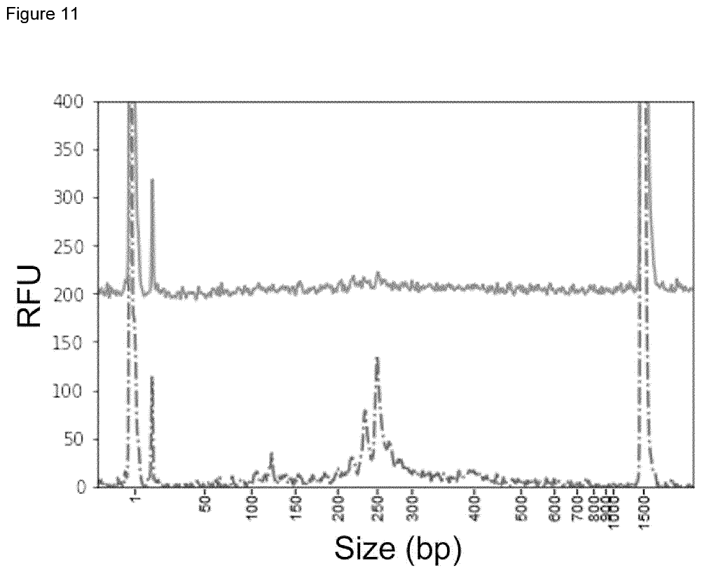

FIG. 11 shows the electropherograms of the two assemblies as obtained after the purification procedure. The symmetric assembly fails, since there is no clear peak at the target length and the target molecule could not be recovered even with purification. In contrast, the asymmetric assembly leads to results where the target molecule can be recovered after purification as seen in a clear peak.

Optionally, the successful construct can be isolated from the gel by using standard kits (e.g., Zymoclean), amplified by PCR (e.g., Sambrook and Russell, 2014; Chapter 8) and sequenced.

TABLE 2
Experimental yield and purity of polynucleotides with
pooled, symmetric and asymmetric assembly methods.

| Assembly | Purity (% Conc) | Yield (ng/μL) |
|---|---|---|
| Tiered | <1.0 | <0.1 |
| Pooled | 7.6 | 0.1 |
| Asymmetric | 40.0 | 0.37 |

### Example 5. Assembly Tree Optimisation of a GC-Rich Target Molecule of 1028 bp with an Asymmetric Workflow by Ligation of Oligonucleotides of Different Lengths Using Liquid Handlers

In this example, the advantage of using an asymmetric workflow and optimization algorithms to partition molecules with challenging sequences into building blocks (oligos) that are of unequal length. The SOI (Seq_03, SEQ ID NO:60) is of 1028 bp and is partitioned in two ways: into fixed-length 16 mers and with a size adaptive between 15 and 17 nt. The scores of the balanced (symmetric) assembly tree for the naïve partition are compared to the optimised assembly trees of both the naïve and the adaptive partitions.

### Choosing the Objective Function

In order to score an assembly tree, a “Misligation Matrix” (M_{i,j}) is generated. Each element of this matrix is 1 if the corresponding overhang pair is exposed during the ligation process, as a consequence of the chosen assembly tree, but it is not supposed to ligate; 0 otherwise. The ligation matrix (L_{i,j}) assigns a score (between 0 and 4000) directly related to the chance of ligation of the corresponding pair, irrespective of whether they are exposed during the assembly process. High GC content pairs have a larger score. Specifically, if the two overhangs have at least 3 out of 4 matching nucleotides according to Watson-Crick pairing, the matrix element is increased by a factor 10 for every A/T pair and by a factor 1000 for every G/C pair. To calculate the score, the two matrices are multiplied, element-wise, and then the total sum of each element is taken.

Overall, the score is:

$$S = - ( {\sum }_{i , j} {L}_{i , j} \times {M}_{i , j} ) / {n}^{2}$$

Where n is the total number of overhangs and the minus sign is necessary to make sure that assembly trees with more misligation propensity have lower scores.

### Partitioning the Sequence and Selecting the Assembly Tree

By using the score defined in the previous section an optimal partition and assembly tree is computed under three different conditions.

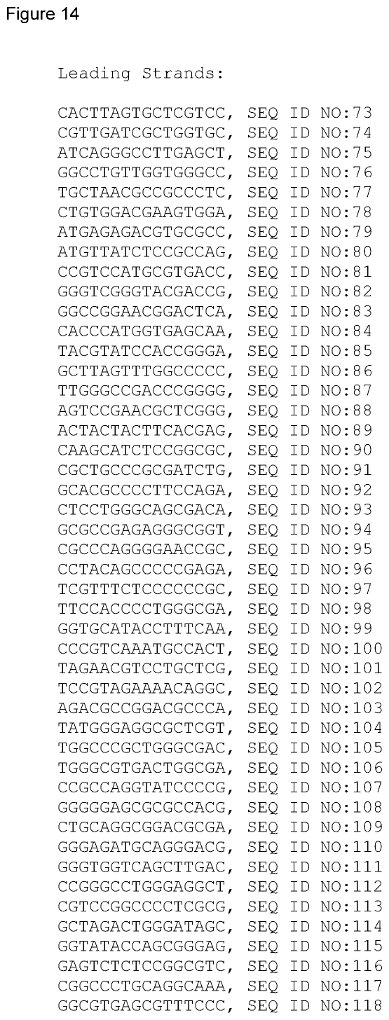

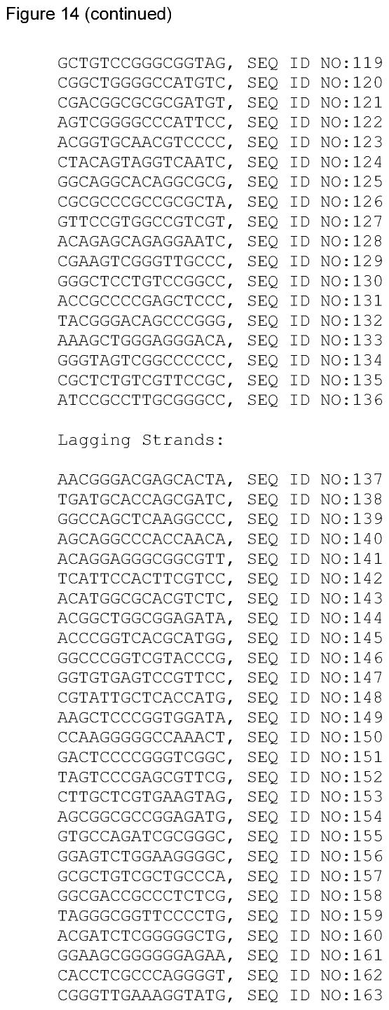

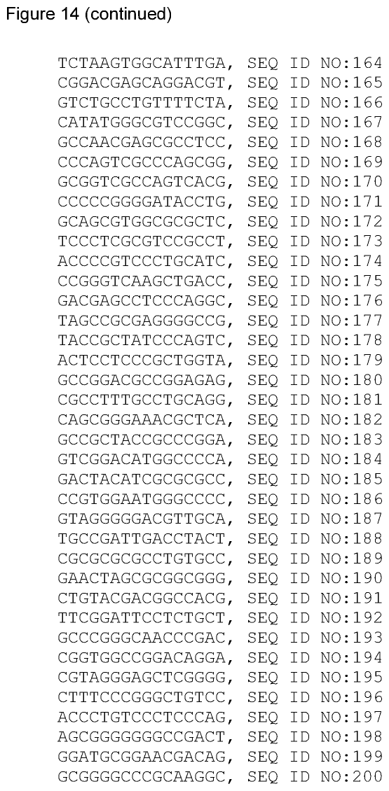

First, a uniform, “naïve” partition is done by segmenting the SOI and its reverse complement into 16 mers (with 4 nt offset to allow for 4 nt 5′ overhangs). The list of oligo sequences is provided in FIG. 14. A symmetric tree is used ad hoc as an assembly workflow. The score is parsed on this partition and topology. The naïve partition resulted in 128 oligonucleotide sequences.

Second, the same naïve partition above is used, but an asymmetric tree is computed in order to minimise misassemblies by means of maximising the score.

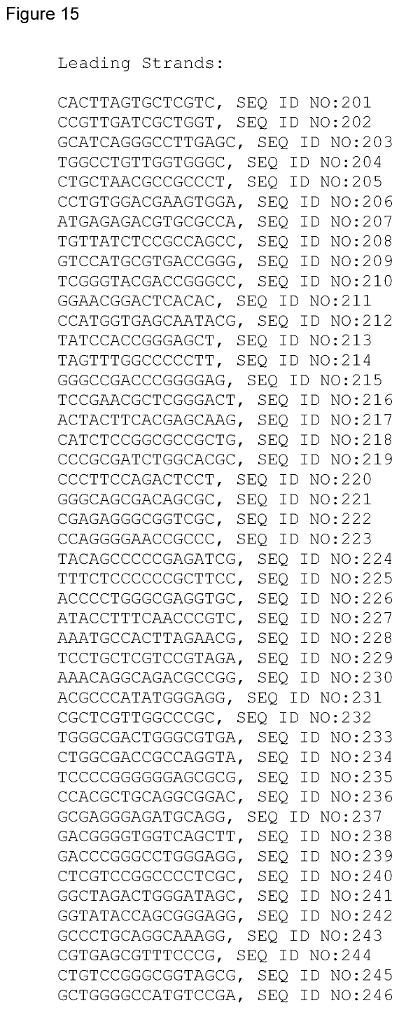

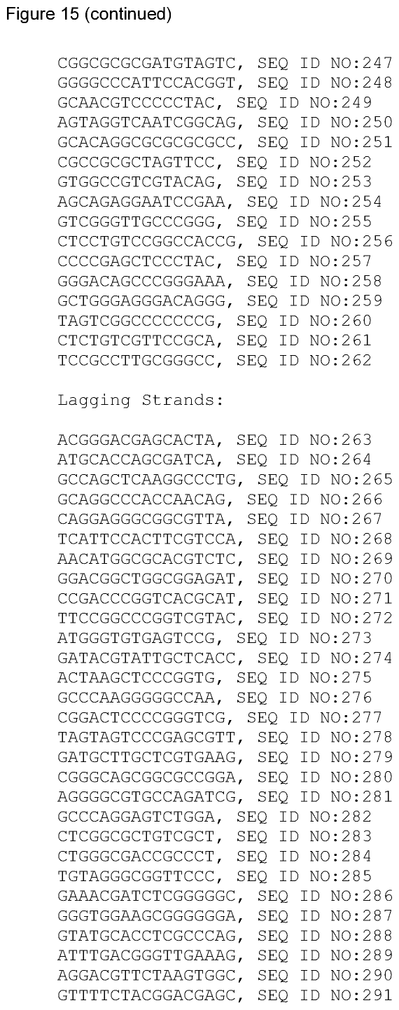

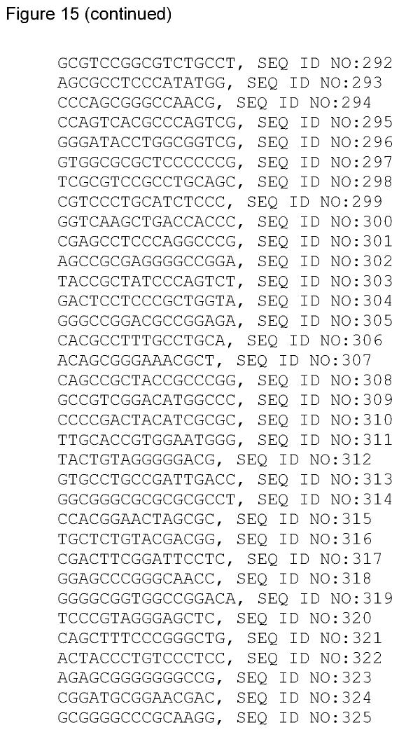

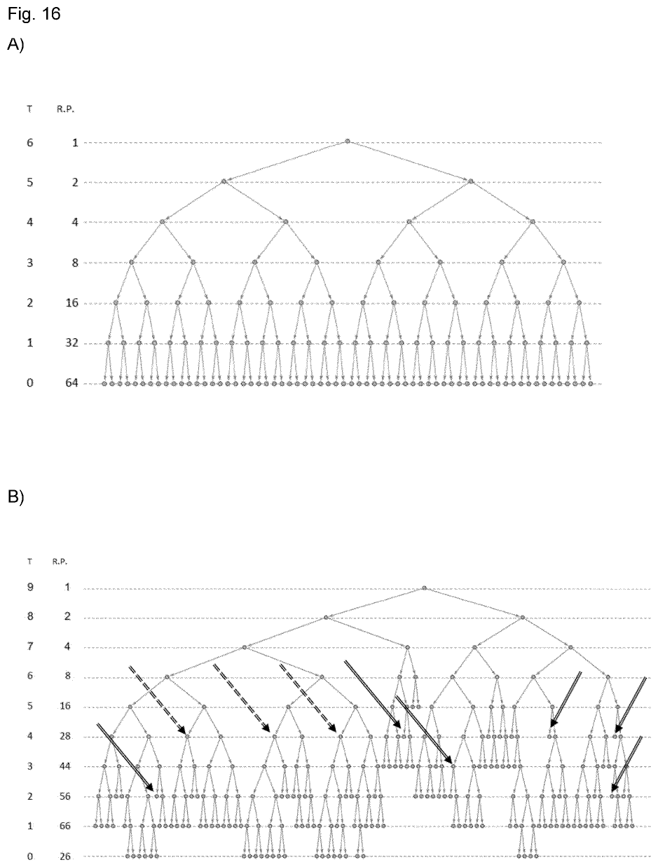

Third, an adaptive partition is done by locally maximising the score. Oligonucleotide sequences range between 15 and 17 nt and each dimer is constrained to have to 4 nt 5′ overhangs. This partition resulted in 126 oligonucleotide sequences. The list of oligo sequences is in FIG. 15. After a locally-optimal partition has been completed, an optimal tree is computed by further maximising the score.

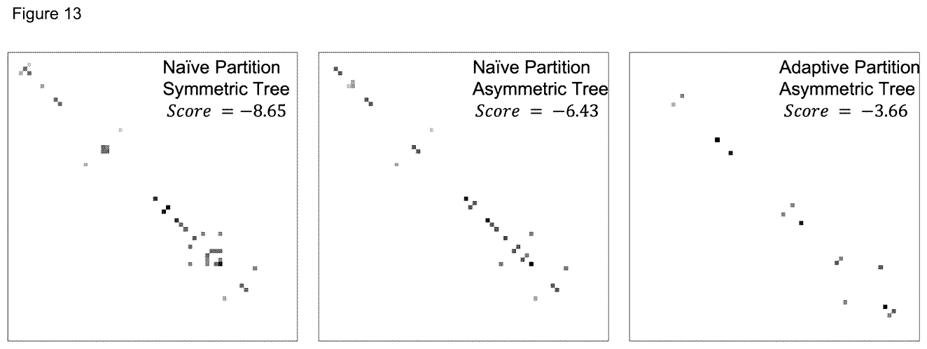

FIG. 12 shows three scored assembly trees, namely a naïve partition with a symmetric tree, a naïve partition with the asymmetric tree and an adaptive partition with a symmetric tree. FIG. 13 shows a graphical representation of the product of the ligation and misligation matrix.

As it appears evident from the scoring, the best result is obtained when the adaptive partitioning method meets the asymmetric assembly tree. In this case the score is increased by more than a factor two. The choice of an asymmetric tree implies additional steps are required for the assembly, but it is largely compensated by the extremely increased chances of a successful assembly, even more so for a sequence like the SOI that has a GC content larger than 60%.

### Provision of the Oligonucleotides

The synthesis of the oligos was outsourced to an established genomic company. The oligo providers were contacted to ensure that arraying included empty wells as required.

For the oligos from the naïve partition with symmetric tree, 1 plate of 364 micro wells was procured, reflecting the systematic order of the oligos (see Example 2) for the symmetric assembly.

The oligos of the naïve partition with an asymmetric tree were procured in 1 plate of 364 micro wells. Even though these are the same oligos as in the symmetric tree, we chose to acquire the oligos readily arrayed and avoid potential mistakes. Similar as in Example 4, the arraying was done by re-casting the oligos into a larger symmetric tree and adding zeromers to the rightmost unassigned terminal branches of the tree. The same logic and procedure were followed to procure the oligos of the adaptive partition and asymmetric tree.

### Assembly of the Sequences

Once oligonucleotides have been provided, these were annealed as in Examples 2-4 to form dimers with 5′ overhangs.

The assembly steps were implemented through symmetric movement of and MCA arm on a Tecan Fluent under the same conditions as in Examples 2-4.

Comparison of the results by means of capillary electrophoresis indicated that the naïve assembly with the symmetric tree failed (no detectable peak). The naïve and adaptive partitions with asymmetric trees both resulted in detectable peaks in the electropherogram. The yield and purity proved to be a higher for the assembly based on the adaptive partition with asymmetric tree than on the naïve partition with asymmetric tree.

A PCR amplification of the target product was performed to enrich the sample. After amplification and clean-up with a Zymoclean kit, sequencing was performed on an Oxford Nanopore minION. The analysis of sequencing results indicate that the assembly is correct.

### Assembly on Acoustic Dispensers

In a different embodiment, the transfer of oligonucleotides can be achieved by means of acoustic transfer (Echo Liquid Handler 525, Beckman-Coulter). In this embodiment, the arraying with zeromers is unnecessary and it is possible to maximise use of the micro-well plates. Instead of mapping a tree into an array that is to be handled with symmetric movements of a multi-channel pipette, the mapping is performed as “worklist”, i.e. a set of instructions (for example in XML format) that indicate the precise order of transfers to be done. Whilst this is not strictly parallel, the accelerated speed of acoustic dispensing makes it effectively parallel, e.g., as compared to the required incubation time of the reactions.

### Example 6. Exemplary Materials and Features that May be Included in an Asymmetric Assembly Using Solid-Support Attachment

As a means to improve the yield of the assemblies, it may be convenient to use purification methods after some or every ligation or assembly reaction. Embodiments may use attachment of the oligonucleotides to a solid support. The oligos may have amino modifications in the 3′ or 5′ ends allowing a covalent or non-covalent bond to an adaptor or to a surface moiety. Solid support may comprise different treatments allowing stable or covalent links through carbonyl amide, carboxamide, sulfonamide or thiourea.

In some embodiments, nucleic acid polynucleotides of 50 or more nt or bp may be attached to solid supports by covalent modification of terminal phosphate groups to cellulose or polysaccharides. Other embodiments for attaching shorter oligonucleotides (20-50 nt) may use using phosphoramidate or amide linkages.

Some embodiments may use proteins or glicoproteins such as avidin, streptadivin or similar variants as part of the solid support to facilitate a non-covalent attachment to a biotin terminal modification. The biotin may be directly attached to the oligo/polynucleotides, thereby establishing a stable, non-covalent interaction. This can be reversed by incubating at high temperatures. Some embodiments use a light-sensitive release chemical moiety in the biotin, allowing cleaving of the covalent bond by exposure to the appropriate wave-length.

### Partitioning the Sequence and Selecting the Assembly Tree

Partitioning of a target sequence is done as in Example 1 and this process is not influenced by the choice of any strategy for solid support and required modifications.

### Provision of the Oligonucleotides on a Solid Support

Some or all oligonucleotides may be provided attached to a solid support by directly deriving products from column chemical synthesis by means of phosporamidite chemistry, or by means of enzymatic synthesis using native or modified TdTs, amongst other enzymatic methods of DNA synthesis. In some embodiments oligonucleotides may be provided on a solid-support, where each oligo is in a separate compartment in a microwell plate with a treated surface allowing stable or covalent links. Some embodiments may attach the oligonucleotides to magnetic beads provided on separate compartments. Other embodiments may attach oligonucleotides to treated glass surfaces forming microarrays.

### Preparation of the Assembly Through Annealing or Polymerase Extension

In some embodiments, the provided oligonucleotide building blocks may be converted into double stranded oligonucleotides by hybridisation of the attached oligos with other single stranded oligos that are in solution and may have no modifications. The hybridisation may be with oligos of equal or similar sizes so that the hybridised oligo results in blunt ends or may leave an overhang. The annealing may be done by controlling the annealing temperature with a thermocycler.

In other embodiments the annealing may be done with DNA primers in order to create a double stranded by elongation using a polymerase such as TAQ or Klenow, thereby forming double stranded DNA or DNA-RNA hybrid.

After hybridisation or elongation, the sample may be subject to washing steps to eliminate unreacted oligonucleotides, cofactors and enzymes.

### Detachment and Assembly of Oligonucleotides and Polynucleotides

Detachment methods depend on the chemical nature of the attachment and on the modification used on the oligonucleotide building blocks. In some embodiments, the detachment may use sequence-dependent enzymatic methods such as restriction enzymes to cleavage the DNA in specific sequences included in an attachment adaptor.

Another embodiment may use detaching a biotin-modified oligo or polynucleotide be means of thermal methods.

Since the presence of biotin may interfere in the assembly, or the use of thermal detachment may disrupt the secondary structure of the ds polynucleotide, some embodiments may use photo-cleavable linkages e.g., amino C6 (5′ end) modifications covalently bonded either to the solid support or to biotin (Olejnik et al 1998). In this case, cleavage is achieved by exposure of the sample to long-wave UV light in the wave length range of 300-350, thereby releasing the oligo with a 5′-phosphate group which can be readily used in further chemical or enzymatic assembly steps.

Other detachment methods of DNA molecules are achieved by including a deoxyuridine site (U) at the 3′ terminus as part of the linker to the solid support. The enzyme Uracil DNA glycosylase can then be used to transform the U into an abasic site, which subsequently can be cut using glyeosylate-lyase endonuclease VIII, thereby releasing the DNA.

In any of these or other embodiments, the detachment at any step or tier is done for less than about half of the reaction components and only and exclusively (a) after pooling and reacting specific pairs or intermediate reaction products of oligonucleotides in separate reaction containers indicated by the asymmetric assembly tree, (b) performing a washing step to eliminate unwanted unreacted reagents from said reaction containers, and (c) before the need to transfer the reaction products to another reaction container which may contain another attached oligo or polynucleotide. Any other reaction compartment whose contents are not to be transferred at a specific step—as indicated by the assembly tree—, may be left momentarily attached. This is in comparison to symmetric trees where about half of the contents need to be detached and transferred.

### Example 7. Use of Electro-Wetting on Dielectric Microfluidics Platform to Assemble a Target Polynucleotide Using an Asymmetric Workflow

An equivalent but different embodiment of an asymmetric assembly can be implemented in microfluidics platforms like those of Electro Wetting on Dielectric (EWOD), also known as “digital microfluidics”.

Although the way of handling the liquids is different in different microfluidics platforms, they may allow an even more efficient implementation of complex assembly workflows. Microfluidics have several advantages over standard liquid handling which include but are not limited to low consumable amounts thanks to the small droplet sizes and sparing a multitude of plastic tips that are costly, pollutant, imprecise and potentially pose bottlenecks to the workflow.

In some embodiments, a digital microfluidic platform is used in order to mix and react micro-droplets in a controlled manner. Droplets may be provided in micro-well plates and prepared with the enzymatic milieux as in FIGS. 2-5 plus necessary surfactants that are required to manipulate the droplets by electro-mechanical means. Droplets may be transferred to the EWOD platform by a liquid handler in small amounts, less than 1 μL, preferably 0.1 μL. The assembly tree is translated into digital movements that merge and mix droplets in accordance to each assembly tree. Once droplets are larger than the characteristic size of the EWOD's platform array, these can be combinatorically parallelized to have, effectively, replicas of the assemblies.

In addition, by using appropriate photosensitive probes it is feasible to discard microdroplets that have resulted on misassemblies and to gather data to improve assembly scores.

### REFERENCES

Bonde, M. T., Kosuri, S., Genee, H. J., Sarup-Lytzen, K., Church, G. M., Sommer, M. O. A. and Wang H. H. (2014) Direct Mutagenesis of Thousands of Genomic Targets Using Microarray-Derived Oligonucleotides. ACS Synthetic Biology 4(1):17-22.
Farzadfard F, Lu TK. Synthetic biology. Genomically encoded analog memory with precise in vivo DNA writing in living cell populations. Science. 2014 Nov. 14; 346(6211):1256272. doi: 10.1126/science.1256272. PMID: 25395541; PMCID: PMC4266475.
Gao, X., LeProust, E., Zhang, H., Srivannavit, O., Gulari, E., Yu, P., . . . & Zhou, X. (2001). A flexible light-directed DNA chip synthesis gated by deprotection using solution photogenerated acids. Nucleic Acids Research, 29(22), 4744-4750.
Kai, J., Puntambekar A., Santiago N., Lee S. H., Sehy D. W., Moore V., Han J. and Ahn C. H. (2012) A novel microfluidic microplate as the next generation assay platform for enzyme linked immunoassays (ELISA). Lab Chip, 12(21): 4257-62.
LeProust, E. M., Peck, B. J., Spirin, K., McCuen, H. B., Moore, B., Namsaraev, E., and Caruthers, M. H. (2010) Synthesis of high-quality libraries of long (150 mer) oligonucleotides by a novel depurination controlled process. Nucleic Acids Research, 38(8), 2522-2540.
Neuner, P., Cortese, R., & Monaci, P. (1998). Codon-based mutagenesis using dimer-phosphoramidites. Nucleic acids research, 26(5), 1223-1227.
Sondek, J., & Shortle, D. (1992). A general strategy for random insertion and substitution substoichiometric mutagenesis: coupling of trinucleotide phosphoramidites. Proceedings of the National Academy of Sciences, 89(8), 3581-3585.
Olejnik, J., Krzymanska-Olejnik, E., Rothschild, K. J. (1998) Photocleavable aminotag phosphoramidites for 5′-termini DNA/RNA labeling. Nucleic Acids Res. 26:3572-3576.

1. A method of assembling nucleic acid (NA) building blocks to produce a double stranded (ds) target polynucleotide, comprising assembling NA building blocks according to a workflow comprising multiple assembly tiers, wherein in a hierarchical assembly, at least one asymmetric pair of NA building blocks of different length that originate from different assembly tiers is connected to produce an intermediate polynucleotide in at least one of said multiple assembly tiers, wherein said intermediate polynucleotide is used as a building block to connect further NA building blocks at both ends of said intermediate polynucleotide in one or more further assembly tiers.

2. The method of claim 1, wherein one of the NA building blocks of the asymmetric pair is a reaction product obtained by assembly of NA building blocks in a previous assembly tier, and the other NA building block of the asymmetric pair is a starting NA building block or a reaction product obtained by assembly of NA building blocks in an assembly tier that is different from said previous assembly tier.

3. The method of claim 1, wherein the NA building blocks are oligonucleotides or polynucleotides.

4. The method of claim 1, wherein the NA building blocks are double stranded or single stranded (ss).

5. The method of claim 1, wherein the workflow provides for the production of ds NA building blocks from respective ss NA building blocks.

6. The method of claim 1, wherein the NA building blocks have a length ranging from 8 nucleotides up to 20% of the length of the target polynucleotide.

7. The method of claim 1, wherein assembly is accomplished by connecting matching ss NA building blocks and/or connecting matching overhangs of a pair of ds NA building blocks.

8. The method of claim 1, wherein assembly is by hybridizing complementary nucleotide sequences, or by a ligation reaction which comprises enzymatic, chemical, or an adaptor ligation.

9. The method of claim 8, wherein said ligation reaction is an enzymatic ligation reaction using a DNA ligase an RNA ligase, a polymerase or ribozymes.

10. The method of claim 1, wherein a solid carrier is used to immobilize one or more of said NA building blocks, the target polynucleotide, or one or more intermediate(s) of assembly.

11. The method of claim 1, wherein said NA building blocks are obtained from a library of NA building blocks, which comprises a diversity of NA building blocks, each contained in a separate library containment.

12. The method of claim 11, wherein said library comprises
a) ss NA building blocks, and/or
b) ds NA building blocks.

13. The method of claim 1, wherein said target polynucleotide has a length of at least 132 bps.

14. The method of claim 1, further comprising a step of enriching the target polynucleotide or one or more intermediates of assembly, by polymerase chain reaction (PCR).

15. The method of claim 1, further comprising a step of processing the target polynucleotide or intermediates of assembly by means of enzymatic modification employing restriction enzymes or kinases, or by chemical means, to facilitate further ligation or to facilitate cloning the target polynucleotide into a vector or plasmid.

16. The method of claim 1, wherein one NA building block of said at least one asymmetric pair is a starting NA building block, and said asymmetric pair is assembled in a second or further tier of assembly.

17. The method of claim 1, wherein said hierarchical assembly comprises at least one asymmetric assembly of said at least one asymmetric pair of NA building blocks.

18. The method of claim 9, wherein the DNA ligase is a T3, T4 or T7 DNA ligase.

19. The method of claim 12, wherein said ss NA building blocks have been obtained by de novo synthesis.

20. The method of claim 12, wherein said ds NA building blocks are produced by assembly of respective ss NA building blocks.
